(19) United States
(12) Patent Application Publication
(10) Pub. No.: US 2024/0294460 A1
(43) Pub. Date: Sep. 5, 2024

(54) DEUTERATED ANALOGS AND DERIVATIVES OF 4-BROMO-2,5-DIMETHOXYPHENETHYLAMINE AND USES THEREOF

(71) Applicant: Terran Biosciences Inc., New York, NY (US)

(72) Inventors: Sam CLARK, Miami, FL (US); Matthew Alexander James DUNCTON, Las Vegas, NV (US)

(21) Appl. No.: 18/685,002

(22) PCT Filed: Aug. 19, 2022
(86) PCT No.: PCT/US2022/040927

§ 371 Date: Feb. 20, 2024

Related U.S. Application Data
(60) Provisional application No. 63/313,216, filed on Feb. 23, 2022.
(60) Provisional application No. 63/275,394, filed on Nov. 3, 2021.
(60) Provisional application No. 63/235,541, filed on Aug. 20, 2021.

(51) Int. Cl. C07C 217/60; A61K 31/137

(52) U.S. Cl. CPC C07C 217/60; A61K 31/137; C07B 2200/05

(57) ABSTRACT

Provided herein are deuterated analogs of 4-bromo-2,5-dimethoxyphenethylamine (2C-B) and related compounds and their uses for the treatment of conditions associated with a neurological disease.

### CROSS REFERENCE TO RELATED APPLICATIONS

This application claims the benefit of U.S. Provisional Patent Application No. 63/235,541, filed Aug. 20, 2021, U.S. Provisional Patent Application No. 63/275,394, filed Nov. 3, 2021, and U.S. Provisional Patent Application No. 63/313,216, filed on Feb. 23, 2022, the content of each of which is incorporated by reference herein in its entirety.

### BACKGROUND OF THE INVENTION

Nearly 1 in 5 adults in the United States suffer from mental illness, and over 50% of Americans will be diagnosed with a psychiatric disorder at some point in their lifetime. 1 in 25 Americans is afflicted with severe mental illness, such as major depression, schizophrenia, or bipolar disorder.

### SUMMARY OF THE INVENTION

In one aspect, provided herein are compounds of Formula (I), or a stereoisomer or a pharmaceutically acceptable salt thereof:

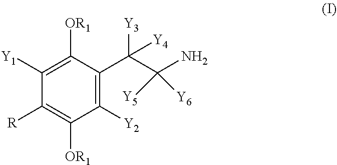

wherein
R is H, D, F, Cl, Br, I, Me, Et, ⁿPr, ⁱPr, ^{c}Pr, ⁿBu, Ph, CH₂Ph, C≡CH (ethynyl), CH═CH₂ (vinyl), CH₂CH═CH₂ (allyl), CH₂OMe, CH₂CF₃, CH₂CH₂F, CHF₂, CF₃, NO₂, NH₂, CN, SeCH₃, OCH₃, OCH(CH₃)₂, SCH₃, SCH₂CH₃, SCH₂C(CH₃)═CH₂, SCH(CH₃)₂, SCH₂CH₂CH₃, SCH₂^{c}Pr, SC(CH₃)₃, SCH₂CH₂OCH₃, SCH₂CH₂SCH₃, S^{c}Pr, SCH₂CH═CH₂, SCH(CH₃)(CH₂CH₃), SCH₂CH₂CH₂CH₃, SCH₂CH₂F, SCH₂CHF₂, SCH₂CF₃, SCH₂CH(CH₃)₂, SCH₂Ph, SCH₂CH₂CH₂F, or SCH₂CH₂CH₂CH₂F;
each R₁ is independently selected from CH₃, CH₂D, CHD₂, and CD₃;
Y₁, Y₂, Y₃, Y₄, Y₅, and Y₆ are each independently H or D; and
at least one R₁ comprises one or more deuterium, or at least one of Y₁, Y₂, Y₃, Y₄, Y₅, and Y₆ is deuterium.

In certain embodiments, R is H or D. In certain embodiments, R is F. In certain embodiments, R is Cl. In certain embodiments, R is Br. In certain embodiments, R is I. In certain embodiments, R is Me. In certain embodiments, R is Et. In certain embodiments, R is ⁿPr. In certain embodiments, R is ⁱPr. In certain embodiments, R is Pr. In certain embodiments, R is ⁿBu. In certain embodiments, R is Ph. In certain embodiments, R is CH₂Ph. In certain embodiments, R is C≡CH. In certain embodiments, R is CH═CH₂. In certain embodiments, R is CH₂CH═CH₂. In certain embodiments, R is CH₂OMe. In certain embodiments, R is CH₂CF₃. In certain embodiments, R is CH₂CH₂F. In certain embodiments, R is CHF₂. In certain embodiments, R is CF₃. In certain embodiments, R is NO₂. In certain embodiments, R is NH₂. In certain embodiments, R is CN. In certain embodiments, R is SeCH₃. In certain embodiments, R is OCH₃. In certain embodiments, R is OCH(CH₃)₂. In certain embodiments, R is SCH₃. In certain embodiments, R is SCH₂CH₃. In certain embodiments, R is SCH₂C(CH₃)═CH₂. In certain embodiments, R is SCH(CH₃)₂. In certain embodiments, R is SCH₂CH₂CH₃. In certain embodiments, R is SCH₂°Pr. In certain embodiments, R is SC(CH₃)₃. In certain embodiments, R is SCH₂CH₂OCH₃. In certain embodiments, R is SCH₂CH₂SCH₃. In certain embodiments, R is S^{c}Pr. In certain embodiments, R is SCH₂CH═CH₂. In certain embodiments, R is SCH(CH₃)(CH₂CH₃). In certain embodiments, R is SCH₂CH₂CH₂CH₃. In certain embodiments, R is SCH₂CH₂F. In certain embodiments, R is SCH₂CHF₂. In certain embodiments, R is SCH₂CF₃. In certain embodiments, R is SCH₂CH(CH₃)₂. In certain embodiments, R is SCH₂Ph. In certain embodiments, R is SCH₂CH₂CH₂F. In certain embodiments, R is SCH₂CH₂CH₂CH₂F.

In certain embodiments, the compound of Formula (I) has a formula of:

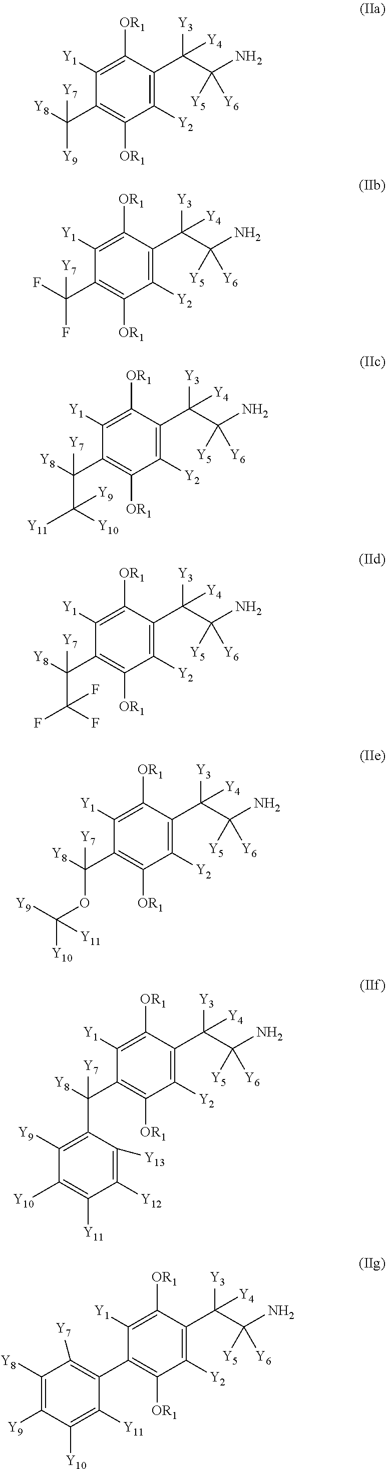

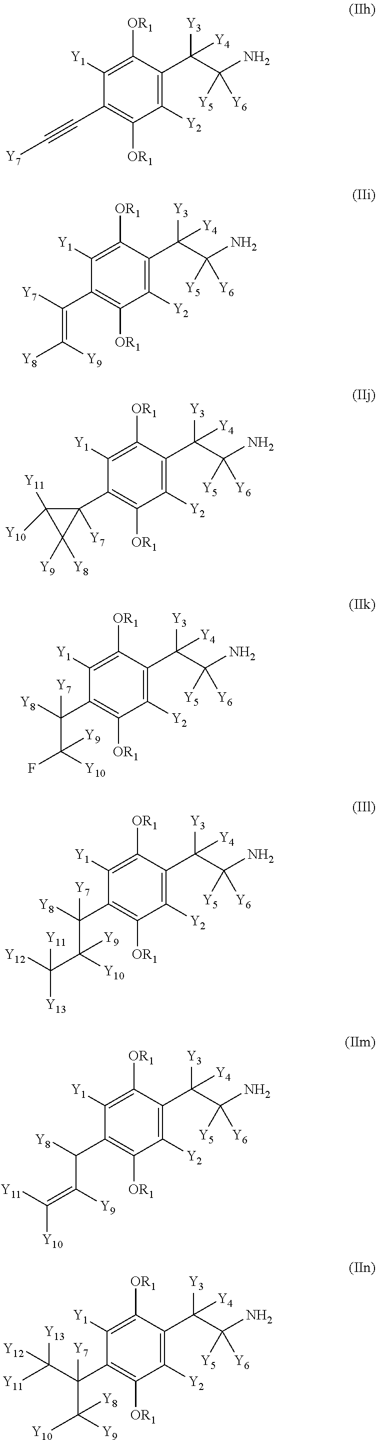

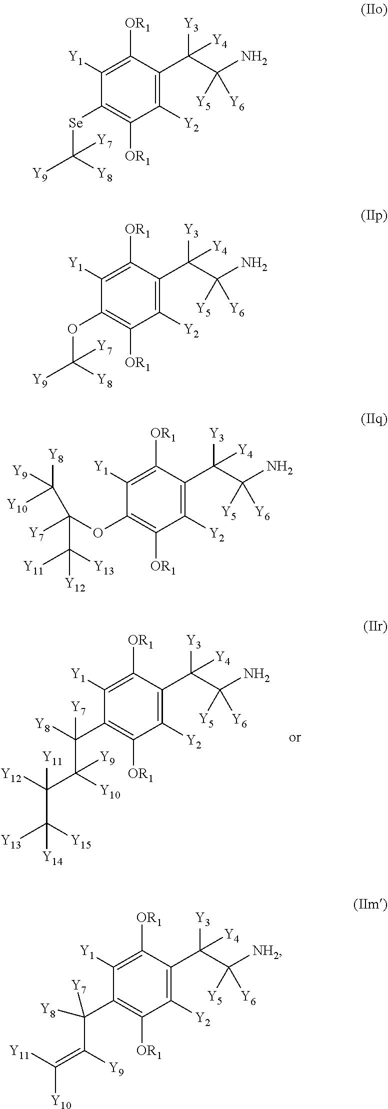

wherein
each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or deuterium; and
wherein at least one R₁ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium.

In certain embodiments, the compound of Formula (I) has a structure of Formula (II):

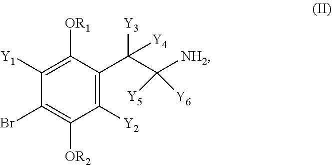

wherein

R₁ and R₂ are each independently selected from CH₃, CH₂D, CHD₂, and CD₃;
Y₁, Y₂, Y₃, Y₄, Y₅, and Y₆ are each independently H or deuterium; and
wherein at least one of R₁ and R₂ comprises one or more deuterium, or at least one of Y₁, Y₂, Y₃, Y₄, Y₅, and Y₆ is deuterium.

In certain embodiments, the compound of Formula (II) has a formula of Formula (II-A):

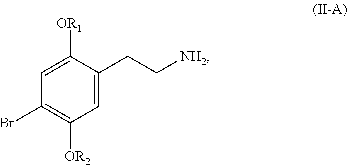

wherein at least one of R₁ and R₂ comprises one or more deuterium.

In certain embodiments, the compound of Formula (II) has a formula of Formula (II-B):

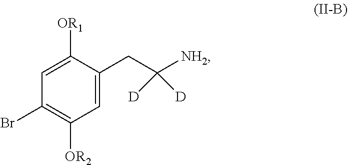

wherein R₁ and R₂ are each independently selected from CH₃, CH₂D, CHD₂, and CD₃.

In certain embodiments, the compound of Formula (II) has a formula of Formula (II-C):

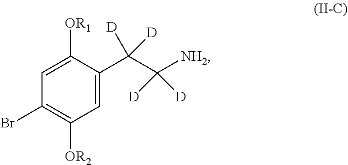

wherein R₁ and R₂ are each independently selected from CH₃, CH₂D, CHD₂, and CD₃.

In certain embodiments, Y₅ and Y₆ are each deuterium, and Y₁, Y₂, Y₃, and Y₄ are each hydrogen.

In certain embodiments, Y₅ is deuterium, and Y₁, Y₂, Y₃, Y₄, and Y₆, are each hydrogen.

In certain embodiments, Y₃, Y₄, Y₅, and Y₆ are each deuterium, and Y₁ and Y₂ are each hydrogen

In certain embodiments, Y₃ and Y₆ are each deuterium, and Y₁, Y₂, Y₄, and Y₅ are each hydrogen.

In certain embodiments, Y₃ and Y₄ are each deuterium, and Y₁, Y₂, Y₅, and Y₆ are each hydrogen.

In certain embodiments, Y₃ is deuterium, and Y₁, Y₂, Y₄, Y₅, and Y₆ are each hydrogen.

In certain embodiments, Y₁, Y₂, Y₃, Y₄, Y₅, and Y₆ are each deuterium.

In certain embodiments, Y₁, Y₂, Y₅, and Y₆ are each deuterium, and Y₃ and Y₄ are each hydrogen.

In certain embodiments, wherein R₁ is CD₃.

In certain embodiments, R₁ is CHD₂.

In certain embodiments, R₁ is CH₂D.

In certain embodiments, R₁ is CH₃.

In certain embodiments, R₂ is CD₃.

In certain embodiments, R₂ is CHD₂.

In certain embodiments, R₂ is CH₂D.

In certain embodiments, R₂ is CH₃.

In certain embodiments, the compound of Formula (II) is selected from the group consisting or:

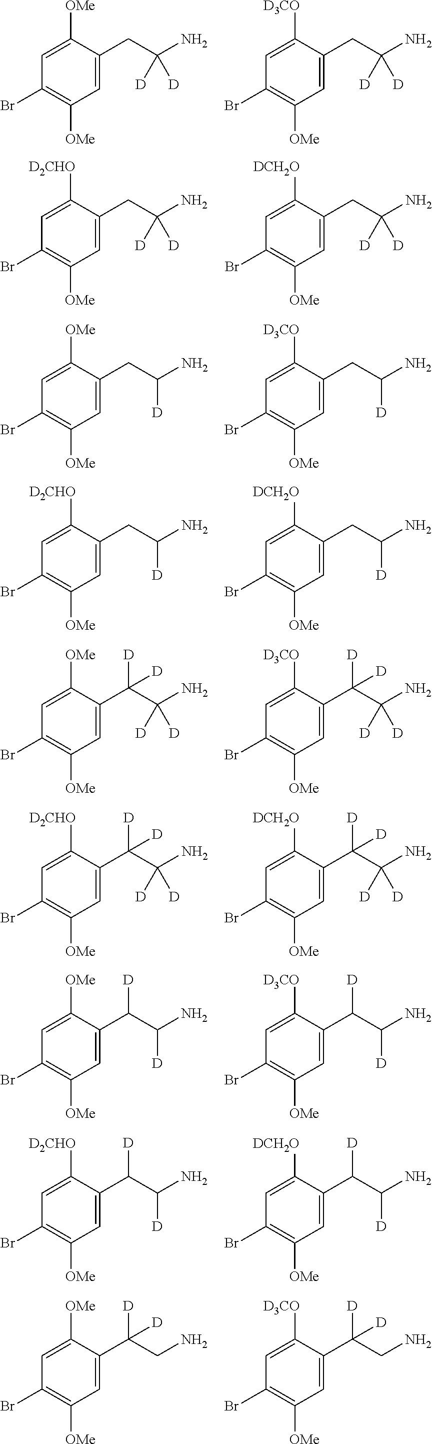

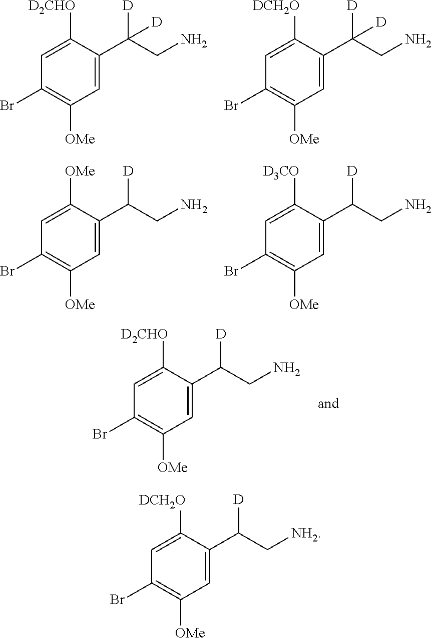

In certain embodiments, the compound of Formula (II) is a compound listed in Table 2.

In certain embodiments, the compound of Formula (II) is

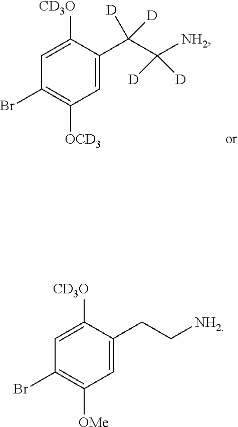

In certain embodiments, the compound of Formula (I) is of Formula (IIm):

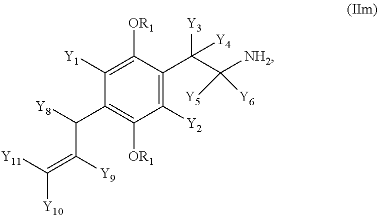

wherein
each R₁ is selected from CH₃, CH₂D, CHD₂, and CD₃;
Y₁, Y₂, Y₃, Y₄, Y₅, Y₆, Y₈, Y₉, Y₁₀, and Y₁₁, when present, are each independently H or deuterium; and
wherein at least one R₁ comprises one or more deuterium, or at least one of Y₁, Y₂, Y₃, Y₄, Y₅, Y₆, Y₈, Y₉, Y₁₀, and Y₁₁ is deuterium.

In certain embodiments, the compound of Formula (I) is of Formula (IIm′):

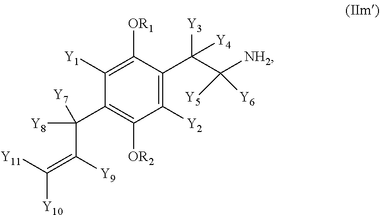

wherein
R₁ and R₂ are independently selected from CH₃, CH₂D, CHD₂, and CD₃;
Y₁, Y₂, Y₃, Y₄, Y₅, Y₆, Y₇, Y₈, Y₉, Y₁₀, and Y₁₁, when present, are each independently H or deuterium; and
wherein at least one of R₁ and R₂ comprises one or more deuterium, or at least one of Y₁, Y₂, Y₃, Y₄, Y₅, Y₆, Y₇, Y₈, Y₉, Y₁₀, and Y₁₁ is deuterium.

In certain embodiments, at least one of R₁ and R₂ comprises one or more deuterium. In certain embodiments, R₁ comprises one or more deuterium. In certain embodiments, R₂ comprises one or more deuterium. In certain embodiments, both R₁ and R₂ comprise one or more deuterium. In certain embodiments, both R₁ and R₂ do not comprise deuterium, and at least one of Y₁, Y₂, Y₃, Y₄, Y₅, Y₆, Y₇, Y₈, Y₉, Y₁₀, and Y₁₁ is deuterium. In certain embodiments, at least 2, 3, 4, 5, 6, 7, 8, 9, 10, or 11 of Y₁, Y₂, Y₃, Y₄, Y₅, Y₆, Y₇, Y₈, Y₉, Y₁₀, and Y₁₁ are deuterium. In certain embodiments, Y₅ and Y₆ are each deuterium, and Y₁, Y₂, Y₃, Y₄, Y₇, Y₈, Y₉, Y₁₀, and Y₁₁ are each hydrogen. In certain embodiments, Y₅ is deuterium, and Y₁, Y₂, Y₃, Y₄, Y₆, Y₇, Y₈, Y₉, Y₁₀, and Y₁₁ are each hydrogen. In certain embodiments, Y₃, Y₄, Y₅, and Y₆ are each deuterium, and Y₁, Y₂, Y₇, Y₈, Y₉, Y₁₀, and Y₁₁ are each hydrogen. In certain embodiments, Y₃ and Y₆ are each deuterium, and Y₁, Y₂, Y₄, Y₅, Y₇, Y₈, Y₉, Y₁₀, and Y₁₁ are each hydrogen. In certain embodiments, Y₃ and Y₄ are each deuterium, and Y₁, Y₂, Y₅, Y₆, Y₇, Y₈, Y₉, Y₁₀, and Y₁₁ are each hydrogen. In certain embodiments, Y₃ is deuterium, and Y₁, Y₂, Y₄, Y₅, Y₆, Y₇, Y₈, Y₉, Y₁₀, and Y₁₁ are each hydrogen. In certain embodiments, Y₁, Y₂, Y₃, Y₄, Y₅, and Y₆ are each deuterium, and Y₇, Y₈, Y₉, Y₁₀, and Y₁₁ are each hydrogen. In certain embodiments, Y₁, Y₂, Y₅, and Y₆ are each deuterium, and Y₃, Y₄, Y₇, Y₈, Y₉, Y₁₀, and Y₁₁ are each hydrogen. In certain embodiments, Y₅, Y₆, Y₇, Y₈, Y₉, Y₁₀, and Y₁₁ are each deuterium, and Y₁, Y₂, Y₃, and Y₄ are each hydrogen. In certain embodiments, Y₅, Y₆, Y₇, and Y₈, are each deuterium, and Y₁, Y₂, Y₃, Y₄, Y₉, Y₁₀, and Y₁₁ are each hydrogen. In certain embodiments, Y₃, Y₄, Y₅, Y₆, Y₇, Y₈, Y₉, Y₁₀, and Y₁₁ are each deuterium, and Y₁ and Y₂ are each hydrogen. In certain embodiments, Y₃, Y₄, Y₅, Y₆, Y₇, and Y₈, are each deuterium, and Y₁, Y₂, Y₉, Y₁₀, and Y₁₁ are each hydrogen. In certain embodiments, Y₆, Y₇, Y₈, Y₉, Y₁₀, and Y₁₁ are each deuterium, and Y₁, Y₂, Y₃, Y₄ and Y₅ are each hydrogen. In certain embodiments, Y₆, Y₇, and Y₈, are each deuterium, and Y₁, Y₂, Y₃, Y₄, Y₅, Y₉, Y₁₀, and Y₁₁ are each hydrogen. In certain embodiments, Y₃, Y₆, Y₇, Y₈, Y₉, Y₁₀, and Y₁₁ are each deuterium, and Y₁, Y₂, Y₅, and Y₄ are each hydrogen. In certain embodiments, Y₃, Y₆, Y₇, and Y₈, are each deuterium, and Y₁, Y₂, Y₄, Y₅, Y₉, Y₁₀, and Y₁₁ are each hydrogen. In certain embodiments, Y₃, Y₄, Y₇, Y₈, Y₉, Y₁₀, and Y₁₁ are each deuterium, and Y₁, Y₂, Y₅, and Y₆ are each hydrogen. In certain embodiments, Y₃, Y₄, Y₇, and Y₈, are each deuterium, and Y₁, Y₂, Y₅, Y₆, Y₉, Y₁₀, and Y₁₁ are each hydrogen. In certain embodiments, Y₃, Y₇, Y₈, Y₉, Y₁₀, and Y₁₁ are each deuterium, and Y₁, Y₂, Y₄, Y₅, and Y₆ are each hydrogen. In certain embodiments, Y₃, Y₇, and Y₈, are each deuterium, and Y₁, Y₂, Y₄, Y₅, Y₆, Y₉, Y₁₀, and Y₁₁ are each hydrogen. In certain embodiments, Y₁, Y₂, Y₅, Y₆, Y₇, Y₈, Y₉, Y₁₀, and Y₁₁ are each deuterium, and Y₃, Y₄ are each hydrogen. In certain embodiments, Y₁, Y₂, Y₅, Y₆, Y₇, and Y₈, are each deuterium, and Y₃, Y₄, Y₉, Y₁₀, and Y₁₁ are each hydrogen. In certain embodiments, Y₁, Y₂, Y₃, Y₄, Y₅, Y₆, Y₇, and Y₈, are each deuterium, and Y₉, Y₁₀, and Y₁₁ are each hydrogen. In certain embodiments, Y₁, Y₂, Y₃, Y₄, Y₅, Y₆, Y₇, Y₈, Y₉, Y₁₀, and Y₁₁ are each deuterium.

In certain embodiments, R₁ is CD₃.

In certain embodiments, R₁ is CHD₂.

In certain embodiments, R₁ is CH₂D.

In certain embodiments, R₂ is CH₃.

In certain embodiments, the compound of Formula (IIm′) is selected from the group consisting of:

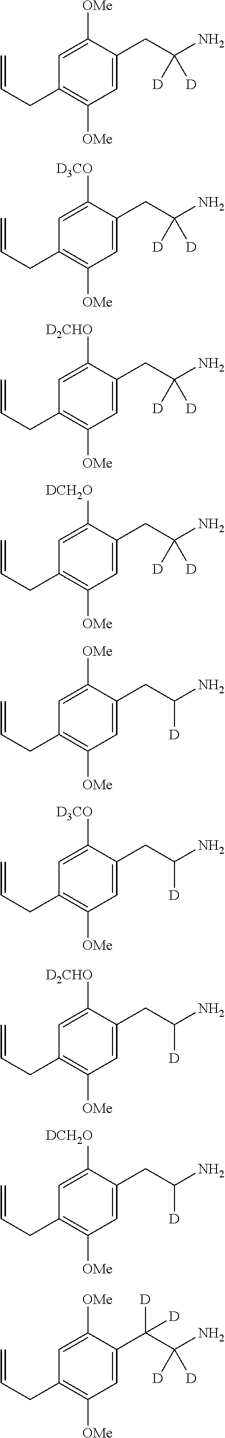

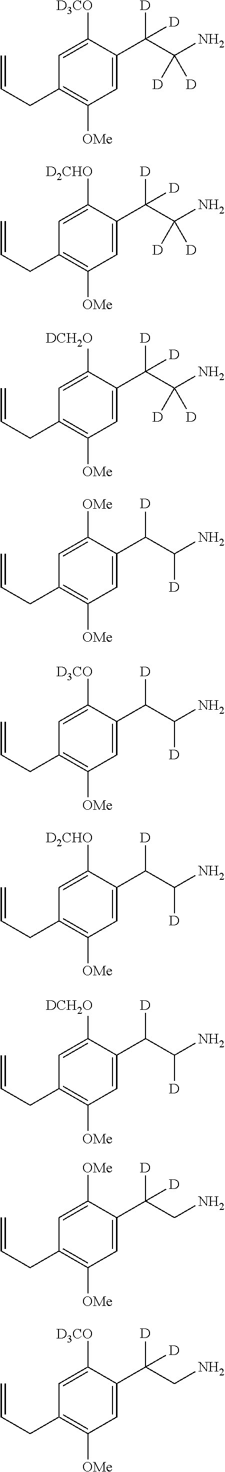

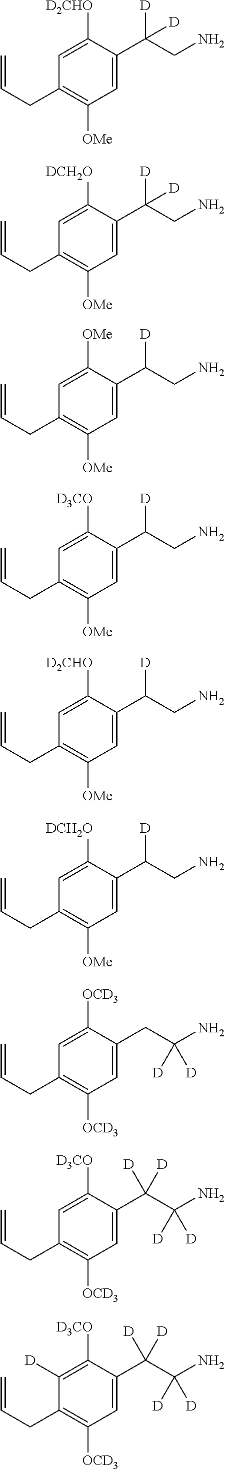

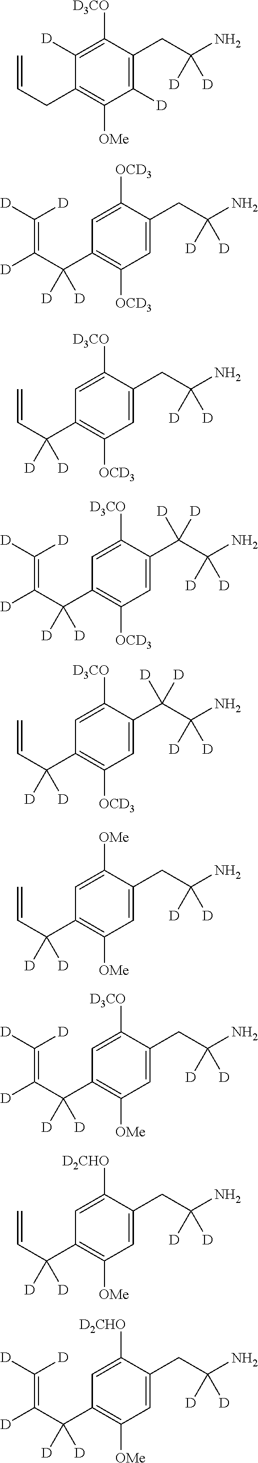

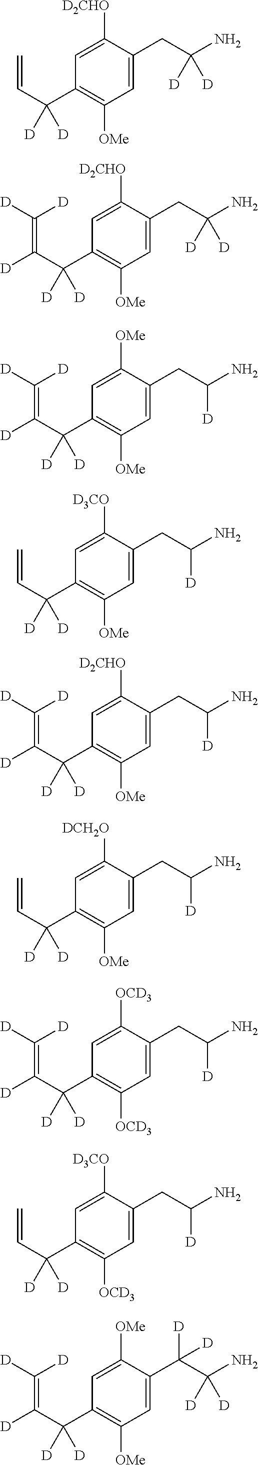

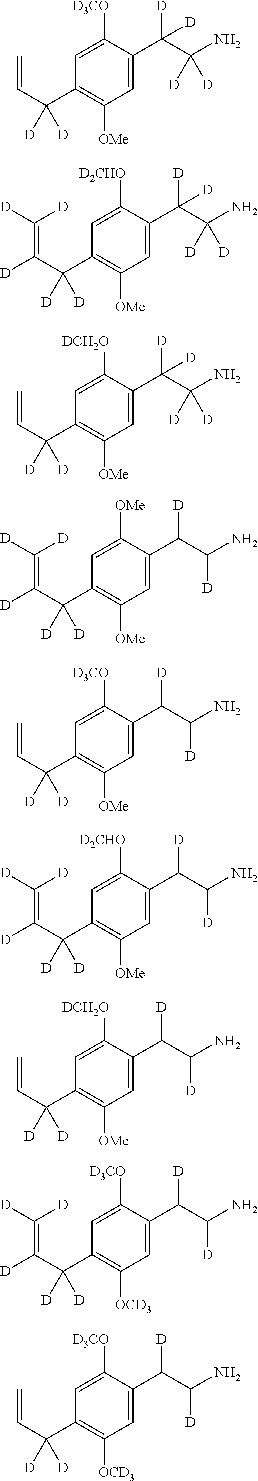

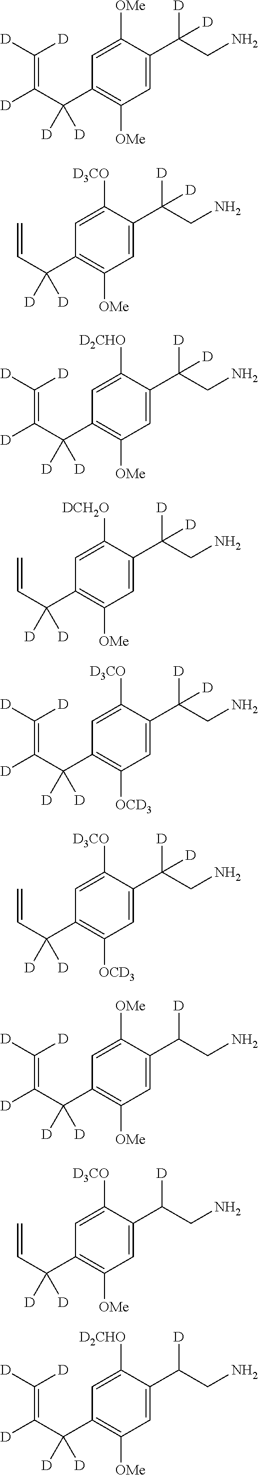

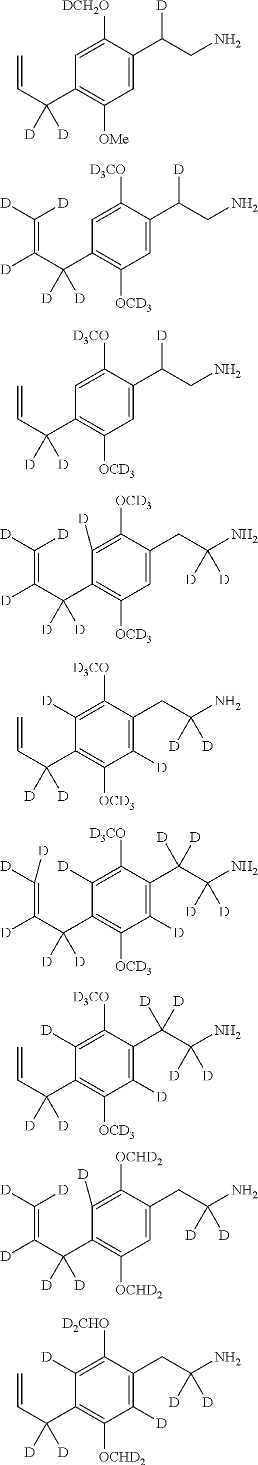

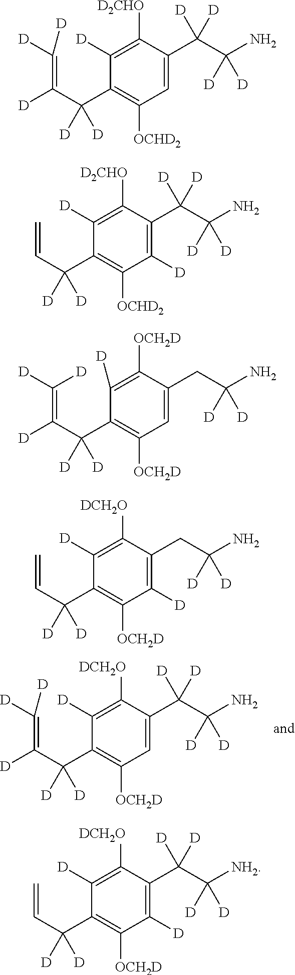

In certain embodiments, the compound has a formula of:

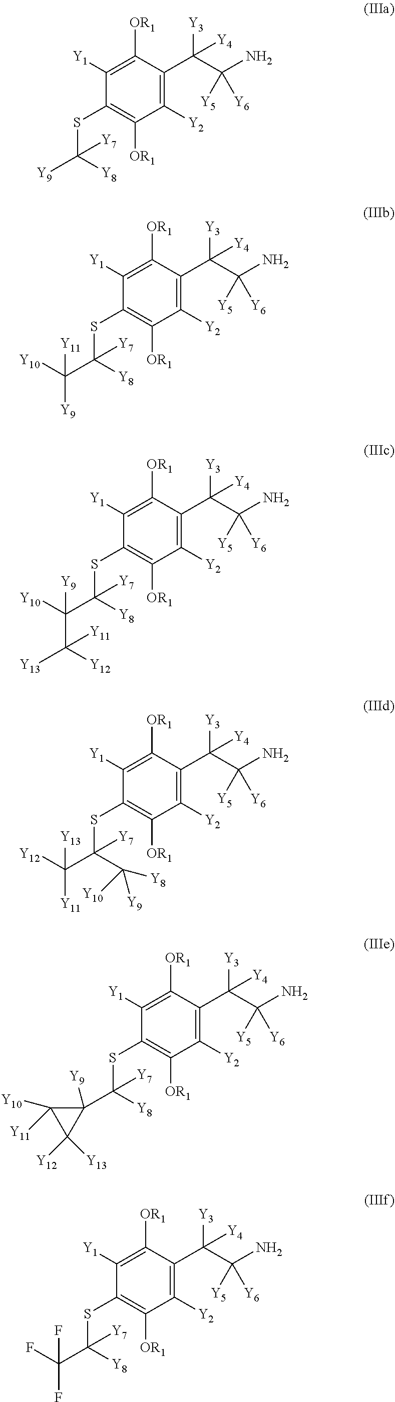

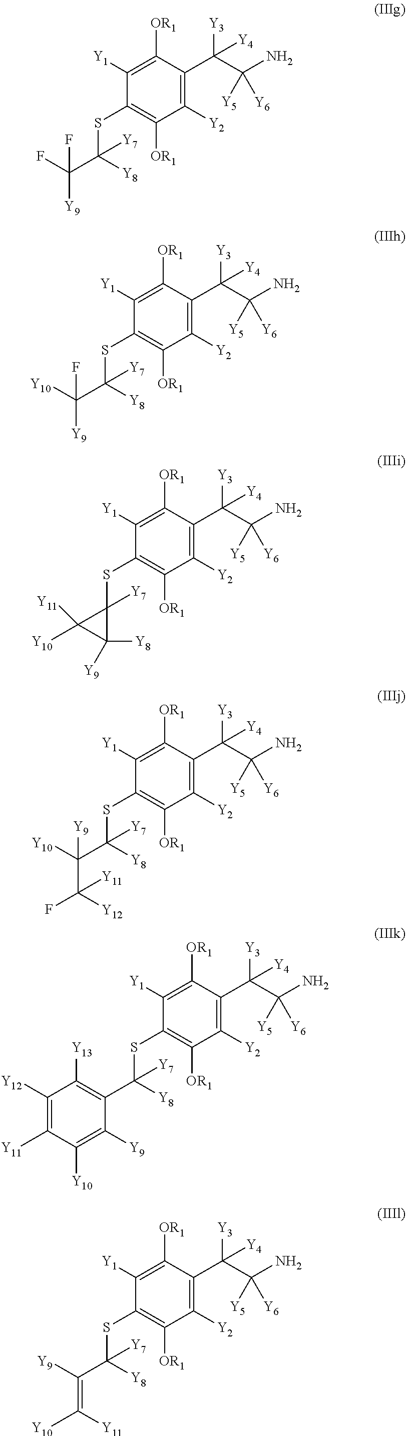

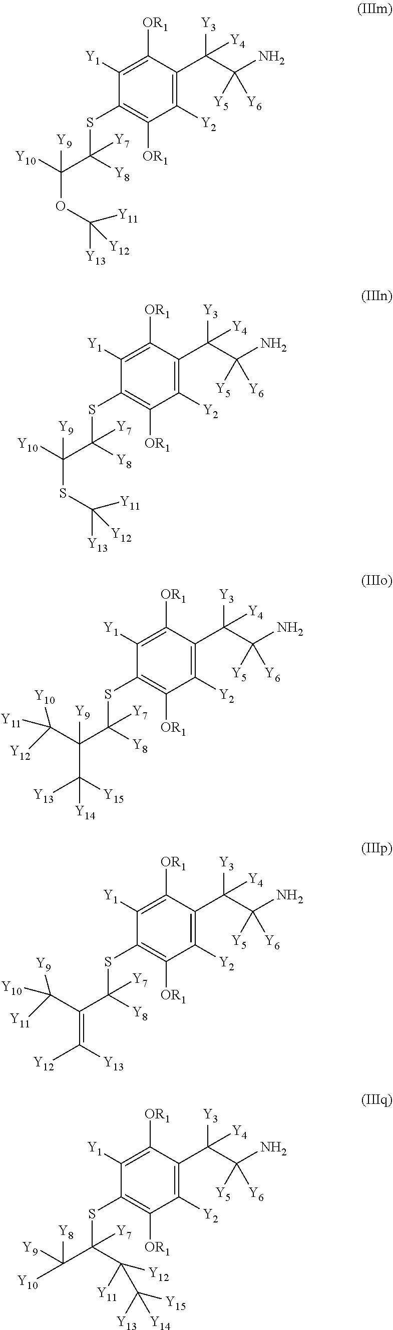

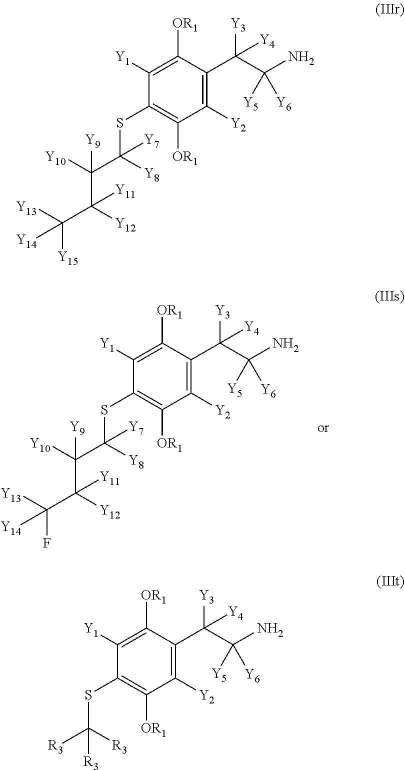

wherein
each R₁ is independently selected from CH₃, CH₂D, CHD₂, and CD₃;
each R₃ is independently selected from CH₃, CH₂D, CHD₂, and CD₃; Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or deuterium; and
wherein at least one of R₁ and R₃ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium.

In certain embodiments, the compound has a formula of:

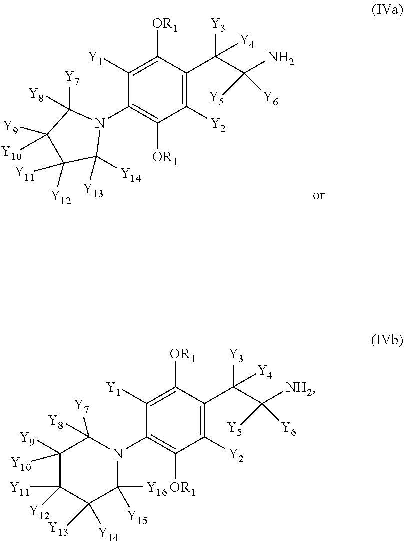

each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆ are each independently H or deuterium; and wherein at least one R₁ comprises one or more deuterium, or at least one of
Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆, when present, is deuterium.

In another aspect, also provided herein are pharmaceutical compositions comprising the compound or stereoisomer or a pharmaceutically acceptable salt described herein, and a pharmaceutically acceptable excipient or carrier.

In yet another aspect, also provided herein are methods for treating or preventing a disease, disorder, or condition a subject in need thereof, comprising administering to the subject an effective amount of the compound or stereoisomer or a pharmaceutically acceptable salt, or the pharmaceutical composition described herein.

In certain embodiments, the disease, disorder, or condition is a brain disease or disorder.

In certain embodiments, the brain disease or disorder is a neurodegenerative disorder.

In certain embodiments, the brain disease or disorder is psychological disorder, depression, addiction, anxiety, or a post-traumatic stress disorder.

In yet another aspect, provided herein are methods for increasing neuronal plasticity in a subject in need thereof, comprising administering to the subject an effective amount of the compound or stereoisomer or a pharmaceutically acceptable salt described herein, or the pharmaceutical composition described herein.

### DETAILED DESCRIPTION

In one aspect, disclosed herein are deuterated analogs of 4-bromo-2,5-dimethoxyphenethylamine (2C-B) and related compounds, e.g. 2-[4-(ethylsulfanyl)-2,5-dimethoxyphenyl]ethan-1-amine (2C-T-2) and 2-(4-iodo-2,5-dimethoxyphenyl)ethan-1-amine (2C-I).

In some embodiments, disclosed herein are compounds of Formula (I):

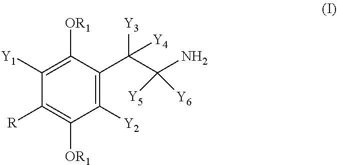

wherein
R═H, D, F, Cl, Br, I, Me, Et, ⁿPr, ⁱPr, ^{c}Pr, ⁿBu, Ph, CH₂Ph, C≡CH (ethynyl), CH═CH₂ (vinyl), CH₂CH═CH₂ (allyl), CH₂OMe, CH₂CF₃, CH₂CH₂F, CHF₂, CF₃, NO₂, NH₂, CN, SeCH₃, OCH₃, OCH(CH₃)₂, SCH₃, SCH₂CH₃, SCH₂C(CH₃)═CH₂, SCH(CH₃)₂, SCH₂CH₂CH₃, SCH₂^{c}Pr, SC(CH₃)₃, SCH₂CH₂OCH₃, SCH₂CH₂SCH₃, S^{c}Pr, SCH₂CH═CH₂, SCH(CH₃)(CH₂CH₃), SCH₂CH₂CH₂CH₃, SCH₂CH₂F, SCH₂CHF₂, SCH₂CF₃, SCH₂CH(CH₃)₂, SCH₂Ph, SCH₂CH₂CH₂F, or SCH₂CH₂CH₂CH₂F;
each R₁ is independently selected from CH₃, CH₂D, CHD₂, and CD₃;
Y₁, Y₂, Y₃, Y₄, Y₅, and Y₆ are each independently H or D; and
at least one R₁ comprises one or more deuterium, or at least one of Y₁, Y₂, Y₃, Y₄, Y₅, and Y₆ is D;
   or a stereoisomer or a pharmaceutically acceptable salt thereof.

### Chemical Abbreviations

H=hydrogen
D=deuterium
Me=methyl or CH₃
Et=Ethyl or CH₂CH₃
ⁿPr=n-propyl or CH₂CH₂CH₃
ⁱPr=isopropyl or CH(CH₃)₂
^{c}Pr=cyclopropyl
ⁿBu=n-butyl or CH₂CH₂CH₂CH₃
C≡CH=ethynyl
CH═CH₂=vinyl
CH₂CH═CH₂=allyl

### Compound Abbreviations

In some embodiments of compounds of Formula (I), R is H (e.g., the compound is an analog of 2C-H) or D.

In some embodiments of compounds of Formula (I), R is F (e.g., the compound is an analog of 2C-F).

In some embodiments of compounds of Formula (I), R is Cl (e.g., the compound is an analog of 2C-C).

In some embodiments of compounds of Formula (I), R is Br (e.g., the compound is an analog of 2C-B).

In some embodiments of compounds of Formula (I), R is I (e.g., the compound is an analog of 2C-I).

In some embodiments of compounds of Formula (I), R is Me (e.g., the compound is an analog of 2C-D).

In some embodiments of compounds of Formula (I), R is Et (e.g., the compound is an analog of 2C-E).

In some embodiments of compounds of Formula (I), R is ⁿPr (e.g., the compound is an analog of 2C-P).

In some embodiments of compounds of Formula (I), R is ⁱPr (e.g., the compound is an analog of 2C-iP).

In some embodiments of compounds of Formula (I), R is ^{c}Pr (e.g., the compound is an analog of 2C-CP).

In some embodiments of compounds of Formula (I), R is ⁿBu (e.g., the compound is an analog of 2C-Bu).

In some embodiments of compounds of Formula (I), R is Ph (e.g., the compound is an analog of 2C-Ph).

In some embodiments of compounds of Formula (I), R is CH₂Ph (e.g., the compound is an analog of 2C-Bn).

In some embodiments of compounds of Formula (I), R is C≡CH (ethynyl) (e.g., the compound is an analog of 2C-YN).

In some embodiments of compounds of Formula (I), R is CH═CH₂ (vinyl) (e.g., the compound is an analog of 2C-V).

In some embodiments of compounds of Formula (I), R is CH₂CH═CH₂ (allyl) (e.g., the compound is an analog of 2C-AL).

In some embodiments of compounds of Formula (I), R is CH₂OMe (e.g., the compound is an analog of 2C-MOM).

In some embodiments of compounds of Formula (I), R is CH₂CF₃ (e.g., the compound is an analog of 2C-TFE).

In some embodiments of compounds of Formula (I), R is CH₂CH₂F (e.g., the compound is an analog of 2C-EF).

In some embodiments of compounds of Formula (I), R is CHF₂ (e.g., the compound is an analog of 2C-DFM).

In some embodiments of compounds of Formula (I), R is CF₃ (e.g., the compound is an analog of 2C-TFM).

In some embodiments of compounds of Formula (I), R is NO₂ (e.g., the compound is an analog of 2C-N).

In some embodiments of compounds of Formula (I), R is NH₂ (e.g., the compound is an analog of 2C-NH₂).

In some embodiments of compounds of Formula (I), R is CN (e.g., the compound is an analog of 2C-CN).

In some embodiments of compounds of Formula (I), R is SeCH₃ (e.g., the compound is an analog of 2C-SE).

In some embodiments of compounds of Formula (I), R is OCH₃ (e.g., the compound is an analog of 2C-O).

In some embodiments of compounds of Formula (I), R is OCH(CH₃)₂ (e.g., the compound is an analog of 2C-O-4).

In some embodiments of compounds of Formula (I), R is SCH₃ (e.g., the compound is an analog of 2C-T).

In some embodiments of compounds of Formula (I), R is SCH₂CH₃ (e.g., the compound is an analog of 2C-T-2).

In some embodiments of compounds of Formula (I), R is SCH₂C(CH₃)═CH₂ (e.g., the compound is an analog of 2C-T-3).

In some embodiments of compounds of Formula (I), R is SCH(CH₃)₂ (e.g., the compound is an analog of 2C-T-4).

In some embodiments of compounds of Formula (I), R is SCH₂CH₂CH₃ (e.g., the compound is an analog of 2C-T-7).

In some embodiments of compounds of Formula (I), R is SCH₂°Pr (e.g., the compound is an analog of 2C-T-8).

In some embodiments of compounds of Formula (I), R is SC(CH₃)₃ (e.g., the compound is an analog of 2C-T-9).

In some embodiments of compounds of Formula (I), R is SCH₂CH₂OCH₃ (e.g., the compound is an analog of 2C-T-13).

In some embodiments of compounds of Formula (I), R is SCH₂CH₂SCH₃ (e.g., the compound is an analog of 2C-T-14).

In some embodiments of compounds of Formula (I), R is S^{c}Pr (e.g., the compound is an analog of 2C-T-15).

In some embodiments of compounds of Formula (I), R is SCH₂CH═CH₂ (e.g., the compound is an analog of 2C-T-16).

In some embodiments of compounds of Formula (I), R is SCH(CH₃)(CH₂CH₃) (e.g., the compound is an analog of 2C-T-17).

In some embodiments of compounds of Formula (I), R is SCH₂CH₂CH₂CH₃ (e.g., the compound is an analog of 2C-T-19).

In some embodiments of compounds of Formula (I), R is SCH₂CH₂F (e.g., the compound is an analog of 2C-T-21).

In some embodiments of compounds of Formula (I), R is SCH₂CHF₂ (e.g., the compound is an analog of 2C-T-21.5).

In some embodiments of compounds of Formula (I), R is SCH₂CF₃ (e.g., the compound is an analog of 2C-T-22).

In some embodiments of compounds of Formula (I), R is SCH₂CH(CH₃)₂ (e.g., the compound is an analog of 2C-T-25).

In some embodiments of compounds of Formula (I), R is SCH₂Ph (e.g., the compound is an analog of 2C-T-27).

In some embodiments of compounds of Formula (I), R is SCH₂CH₂CH₂F (e.g., the compound is an analog of 2C-T-28).

In some embodiments of compounds of Formula (I), R is SCH₂CH₂CH₂CH₂F (e.g., the compound is an analog of 2C-T-30).

### Compounds of Formula (I)

In some embodiments, when R₁ is a substituent that contains one or more hydrogen, any of such one or more hydrogens in R₁ can also be replaced by deuterium. In some embodiments, the compound of Formula (I) has the following formula:

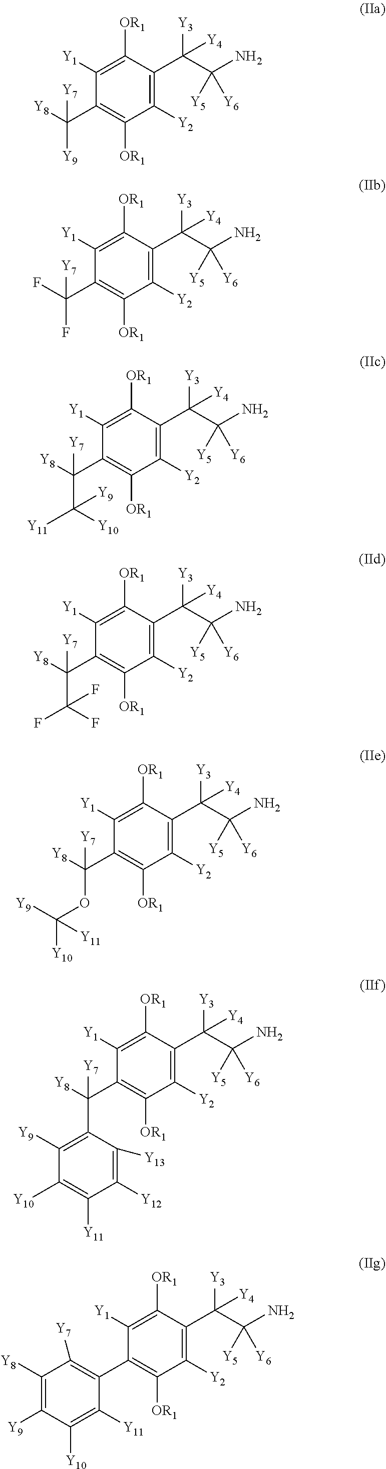

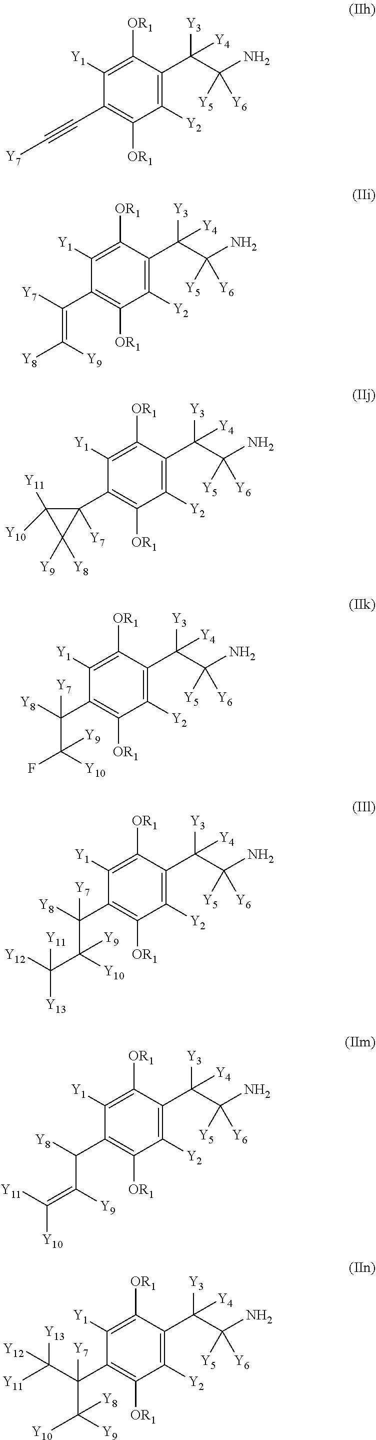

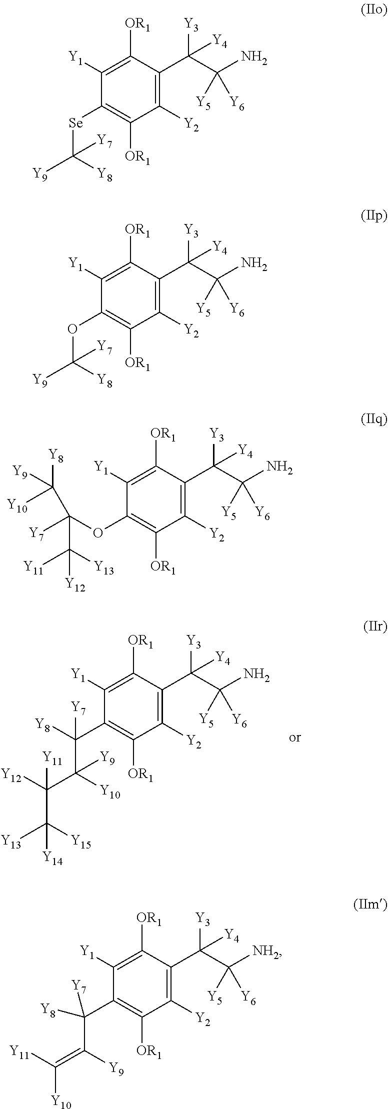

wherein
each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or D; and
wherein at least one R₁ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium;
or a stereoisomer or a pharmaceutically acceptable salt thereof.

In some embodiments, the compound of Formula (I) is of Formula (II):

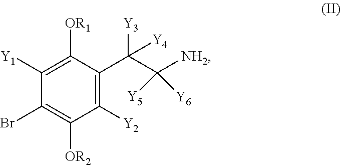

wherein:
each of R₁ and R₂ is independently selected from CH₃, CH₂D, CHD₂, and CD₃;
Y₁, Y₂, Y₃, Y₄, Y₅, and Y₆ are each independently H or D; and
wherein at least one R₁ comprises one or more deuterium, or at least one of Y₁, Y₂, Y₃, Y₄, Y₅, and Y₆ is deuterium;
   or a stereoisomer or a pharmaceutically acceptable salt thereof.

In some embodiments, when R₁ is a substituent that contains one or more hydrogen, any of such one or more hydrogens in R₁ can also be replaced by deuterium.

In some embodiments, the compound of Formula (I) has the following formula:

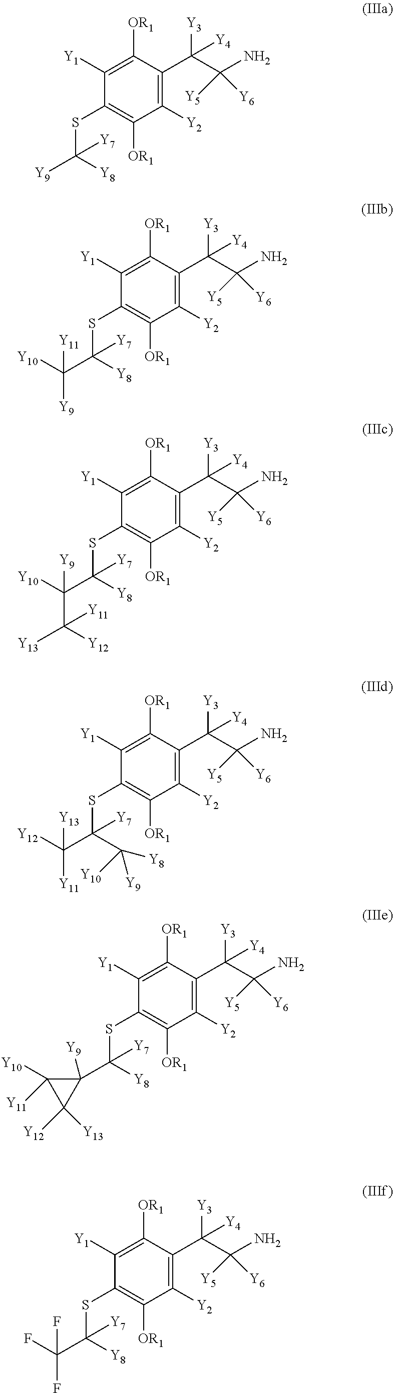

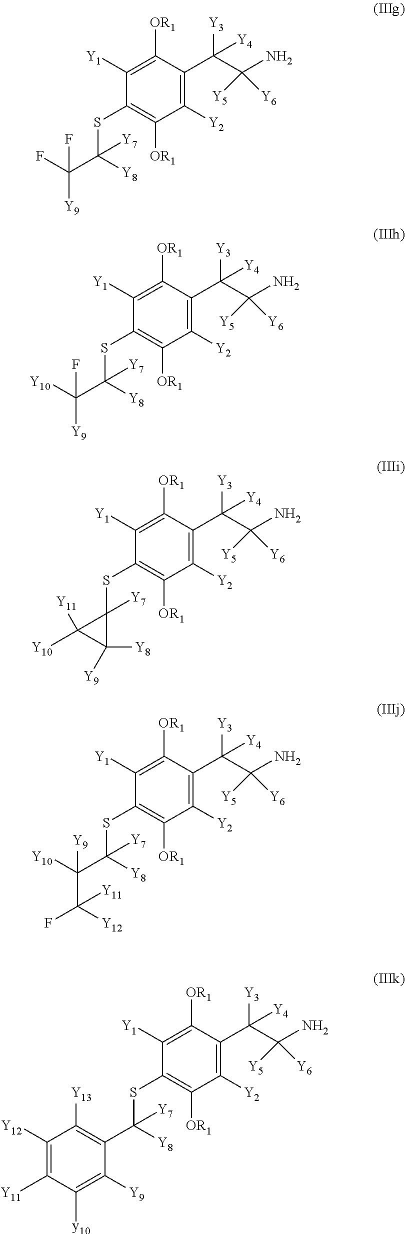

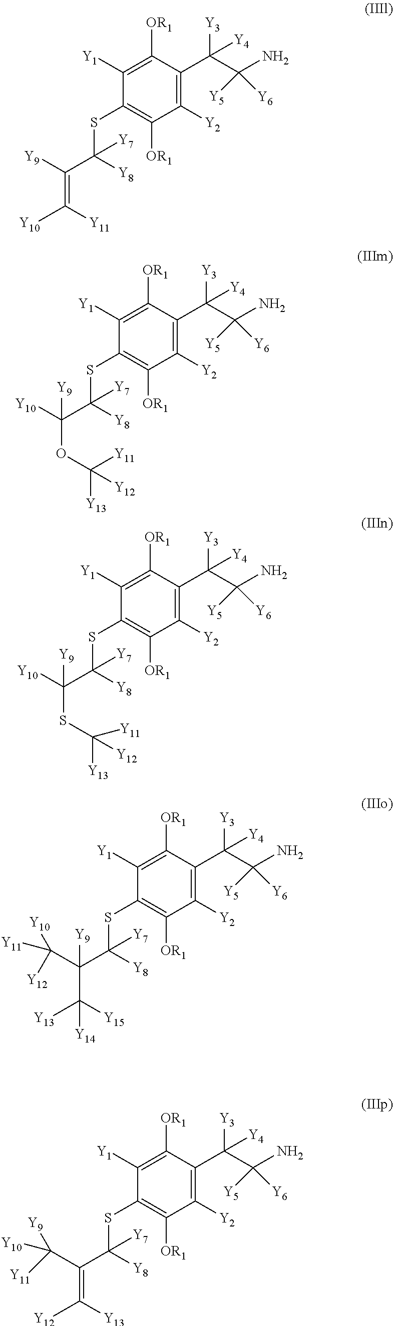

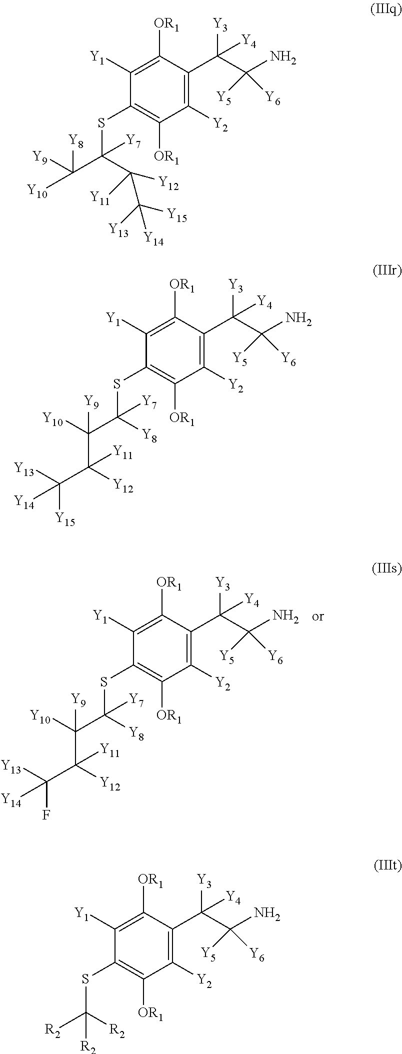

wherein
each R₁ is independently selected from CH₃, CH₂D, CHD₂, and CD₃;
each R₃ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or D; and
wherein at least one of R₁ and R₃ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium;
   or a stereoisomer or a pharmaceutically acceptable salt thereof.

In some embodiments, when R₁ is a substituent that contains one or more hydrogen, any of such one or more hydrogens in R₁ can also be replaced by deuterium.

In some embodiments, the compound of Formula (I) has the following formula:

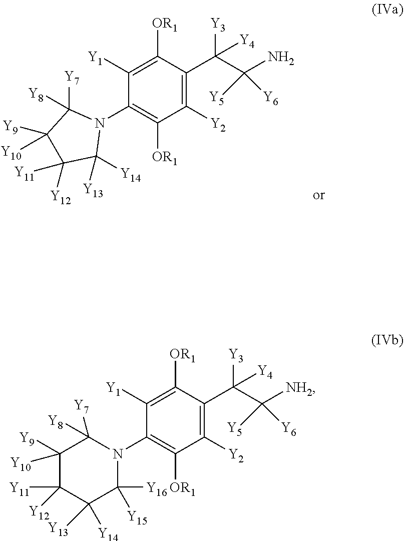

each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆ are each independently H or D; and wherein at least one R₁ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆, when present, is deuterium;
   or a stereoisomer or a pharmaceutically acceptable salt thereof.

In some embodiments, the compound of Formula (I) has the structure of formula (IIm):

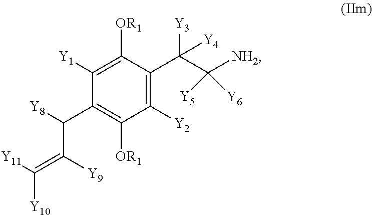

each R₁ is selected from CH₃, CH₂D, CHD₂, and CD₃;
Y₁, Y₂, Y₃, Y₄, Y₅, Y₆, Y₈, Y₉, Y₁₀, and Y₁₁, when present, are each independently H or D; and
wherein at least one R₁ comprises one or more deuterium, or at least one of Y₁, Y₂, Y₃, Y₄, Y₅, Y₆, Y₈, Y₉, Y₁₀, and Y₁₁ is deuterium;
   or a stereoisomer or a pharmaceutically acceptable salt thereof.

In some embodiments, the compound of Formula (I) has the structure of formula (IIm′):

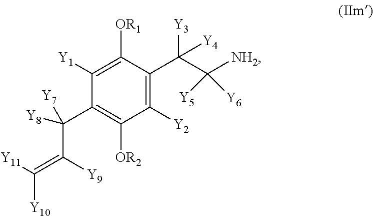

wherein
each R₁ and R₂ is independently selected from CH₃, CH₂D, CHD₂, and CD₃;
Y₁, Y₂, Y₃, Y₄, Y₅, Y₆, Y₇, Y₈, Y₉, Y₁₀, and Y₁₁, when present, are each independently H or D; and
wherein at least one of R₁ and R₂ comprises one or more deuterium, or at least one of Y₁, Y₂, Y₃, Y₄, Y₅, Y₆, Y₇, Y₈, Y₉, Y₁₀, and Y₁₁ is deuterium;
   or a stereoisomer or a pharmaceutically acceptable salt thereof.

In some embodiments, at least one of R₁ and R₂ comprises one or more deuterium. In one embodiment, R₁ comprises one or more deuterium. In one embodiment, R₂ comprises one or more deuterium. In another embodiment, both R₁ and R₂ comprise one or more deuterium.

In some embodiments, both R₁ and R₂ do not comprise deuterium, and at least one of Y₁, Y₂, Y₃, Y₄, Y₅, Y₆, Y₇, Y₈, Y₉, Y₁₀, and Y₁₁ is deuterium.

In some embodiments, at least 2, 3, 4, 5, 6, 7, 8, 9, 10, or 11 of Y₁, Y₂, Y₃, Y₄, Y₅, Y₆, Y₇, Y₈, Y₉, Y₁₀, and Y₁₁ are deuterium. In one embodiment, 2 of Y₁, Y₂, Y₃, Y₄, Y₅, Y₆, Y₇, Y₈, Y₉, Y₁₀, and Y₁₁ are deuterium. In another embodiment, 3 of Y₁, Y₂, Y₃, Y₄, Y₅, Y₆, Y₇, Y₈, Y₉, Y₁₀, and Y₁₁ are deuterium. In another embodiment, 4 of Y₁, Y₂, Y₃, Y₄, Y₅, Y₆, Y₇, Y₈, Y₉, Y₁₀, and Y₁₁ are deuterium. In another embodiment, 5 of Y₁, Y₂, Y₃, Y₄, Y₅, Y₆, Y₇, Y₈, Y₉, Y₁₀, and Y₁₁ are deuterium. In another embodiment, 6 of Y₁, Y₂, Y₃, Y₄, Y₅, Y₆, Y₇, Y₈, Y₉, Y₁₀, and Y₁₁ are deuterium. In another embodiment, 7 of Y₁, Y₂, Y₃, Y₄, Y₅, Y₆, Y₇, Y₈, Y₉, Y₁₀, and Y₁₁ are deuterium. I n another embodiment, 8 of Y₁, Y₂, Y₃, Y₄, Y₅, Y₆, Y₇, Y₈, Y₉, Y₁₀, and Y₁₁ are deuterium. In another embodiment, 9 of Y₁, Y₂, Y₃, Y₄, Y₅, Y₆, Y₇, Y₈, Y₉, Y₁₀, and Y₁₁ are deuterium. In another embodiment, 10 of Y₁, Y₂, Y₃, Y₄, Y₅, Y₆, Y₇, Y₈, Y₉, Y₁₀, and Y₁₁ are deuterium. In another embodiment, all of Y₁, Y₂, Y₃, Y₄, Y₅, Y₆, Y₇, Y₈, Y₉, Y₁₀, and Y₁₁ are deuterium.

In some embodiments, Y₅ and Y₆ are each deuterium, and Y₁, Y₂, Y₃, Y₄, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₅ is deuterium, and Y₁, Y₂, Y₃, Y₄, Y₆, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₅, and Y₆ are each deuterium, and Y₁, Y₂, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ and Y₆ are each deuterium, and Y₁, Y₂, Y₄, Y₅, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ and Y₄ are each deuterium, and Y₁, Y₂, Y₅, Y₆, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ is deuterium, and Y₁, Y₂, Y₄, Y₅, Y₆, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₃, Y₄, Y₅, and Y₆ are each deuterium, and Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₅, and Y₆ are each deuterium, and Y₃, Y₄, Y₇, Y₈, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₅, Y₆, Y₇, Y₈, Y₉, Y₁₀, and Y₁₁ are each deuterium, and Y₁, Y₂, Y₃, and Y₄ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₅, Y₆, Y₇, and Y₈, are each deuterium, and Y₁, Y₂, Y₃, Y₄, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₅, Y₆, Y₇, Y₈, Y₉, Y₁₀, and Y₁₁ are each deuterium, and Y₁ and Y₂ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₅, Y₆, Y₇, and Y₈, are each deuterium, and Y₁, Y₂, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₆, Y₇, Y₈, Y₉, Y₁₀, and Y₁₁ are each deuterium, and Y₁, Y₂, Y₃, Y₄ and Y₅ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₆, Y₇, and Y₈, are each deuterium, and Y₁, Y₂, Y₃, Y₄, Y₅, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₆, Y₇, Y₈, Y₉, Y₁₀, and Y₁₁ are each deuterium, and Y₁, Y₂, Y₅, and Y₄ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₆, Y₇, and Y₈, are each deuterium, and Y₁, Y₂, Y₄, Y₅, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₇, Y₈, Y₉, Y₁₀, and Y₁₁ are each deuterium, and Y₁, Y₂, Y₅, and Y₆ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₇, and Y₈, are each deuterium, and Y₁, Y₂, Y₅, Y₆, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₇, Y₈, Y₉, Y₁₀, and Y₁₁ are each deuterium, and Y₁, Y₂, Y₄, Y₅, and Y₆ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₇, and Y₈, are each deuterium, and Y₁, Y₂, Y₄, Y₅, Y₆, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₅, Y₆, Y₇, Y₈, Y₉, Y₁₀, and Y₁₁ are each deuterium, and Y₃, Y₄ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₅, Y₆, Y₇, and Y₈, are each deuterium, and Y₃, Y₄, Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₃, Y₄, Y₅, Y₆, Y₇, and Y₈, are each deuterium, and Y₉, Y₁₀, and Y₁₁ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₃, Y₄, Y₅, Y₆, Y₇, Y₈, Y₉, Y₁₀, and Y₁₁ are each deuterium.

### Metabolic Pathways of 2C-B

The main metabolic pathways of 2C-B are illustrated in Scheme 1 and Table 1 below (see, e.g., Carmo et al Toxicology 2005, 206, 75-89).

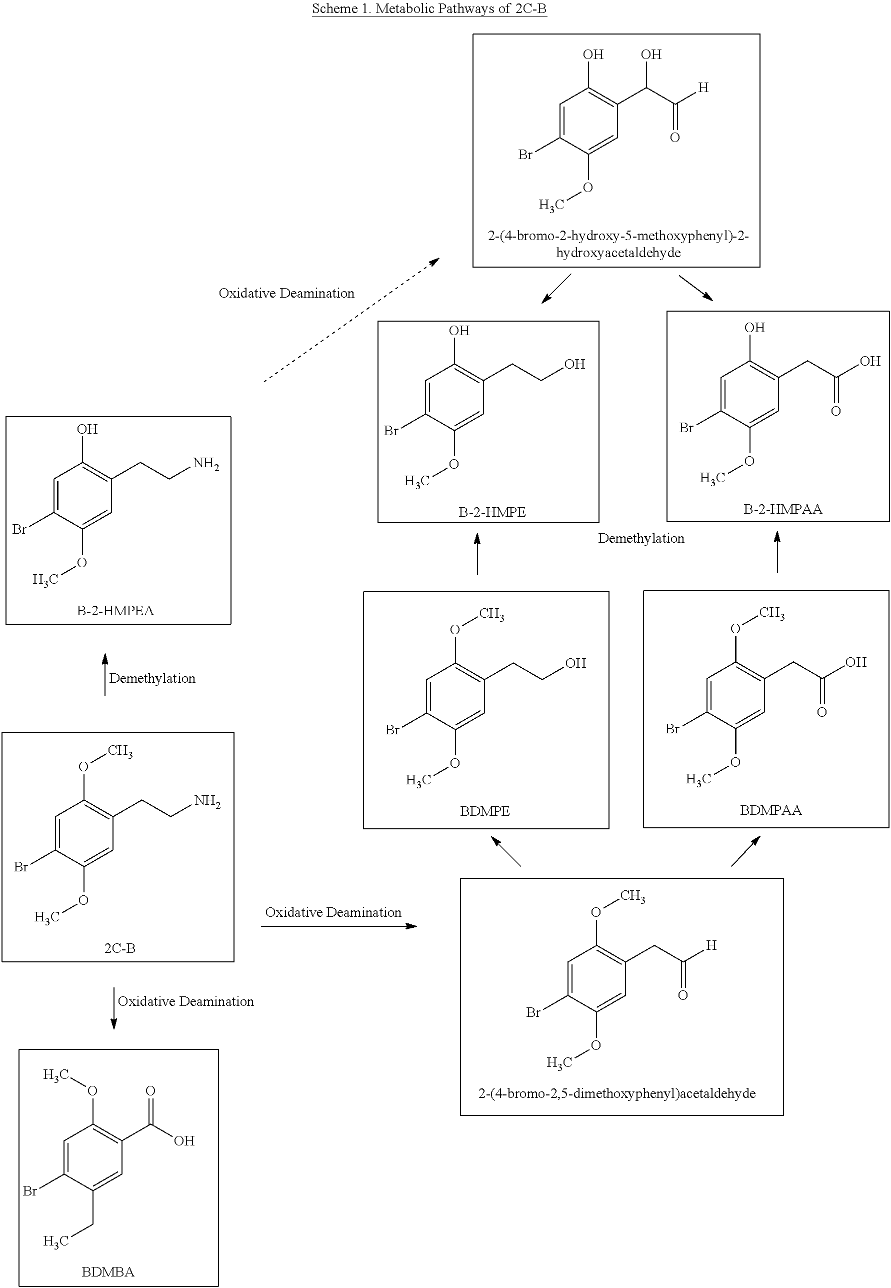

TABLE 1
Formation of 2C-B metabolites by human, monkey, rabbit, mouse, and dog
hepatocytes after incubation with 100 μM and 1000 μM 2C-B

| | | 2C-B (μM) | | | | | | | | | | | |
|---|---|---|---|---|---|---|---|---|---|---|---|---|---|
| | | BDMPAA | | B-2-HMPAA | | BDMBA | | BDMP | | BDMPE | | B-2-HMPE | |
| | Experiment* | 100 | 1000 | 100 | 1000 | 100 | 1000 | 100 | 1000 | 100 | 1000 | 100 | 1000 |
| Human | Donor 3 (fresh) | + | + | + | + | − | + | − | − | + | + | + | − |
| | Donor 2 (fresh) | + | + | + | − | + | + | − | − | + | + | + | − |
| | Donor 2 (cryo) | nd | + | nd | + | nd | + | nd | − | nd | + | nd | − |
| | Donor 1 (cryo) | nd | + | nd | + | nd | + | nd | − | nd | + | nd | + |
| Monkey | 1 | + | + | + | − | + | + | − | − | + | + | + | − |
| | 2 | nd | + | nd | + | nd | − | nd | − | nd | + | nd | − |
| | 3 | + | + | + | − | − | + | − | − | − | − | + | − |
| Rabbit | 1 | + | + | + | + | + | + | − | − | + | + | + | + |
| | 2 | + | + | + | + | + | + | − | − | − | + | + | + |
| | 3 | + | + | + | + | − | + | − | − | − | + | + | + |
| Rat | 1 | + | + | − | − | − | − | − | − | + | + | − | − |
| | 2 | + | + | − | − | − | − | − | − | + | + | − | − |
| | 3 | + | + | + | + | + | + | − | − | − | − | − | − |
| Mouse | 1 | + | + | + | + | − | + | − | + | − | − | − | − |
| | 2 | + | + | + | + | + | + | − | + | − | + | − | − |
| | 3 | + | + | + | − | + | + | − | + | − | − | − | − |
| Dog | | nd | + | nd | − | nd | + | nd | − | nd | + | nd | − |
| sb only | | − | − | − | − | − | − | | | | | | |

nd: not determined; fresh: human freshly isolated hepatocytes; cryo: human cryopreserved hepatocytes.
*Hepatocytes from all species were cryopreserved except for human donors 2 and 3; sb: suspension buffer.

### Deuterated Analogs of 2C-B

In some embodiments, provided herein are deuterated analogs of 2C-B, e.g., compounds of Formula (II):

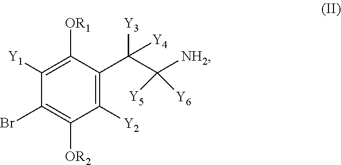

wherein:
R₁ and R₂ are each independently selected from CH₃, CH₂D, CHD₂, and CD₃;
Y₁, Y₂, Y₃, Y₄, Y₅, and Y₆ are each independently H or D; and
wherein at least one of R₁ and R₂ comprises one or more deuterium, or at least one of Y₁, Y₂, Y₃, Y₄, Y₅, and Y₆ is deuterium;
   or a stereoisomer or a pharmaceutically acceptable salt thereof.

In some embodiments of compounds of Formula (II), based on the site and degree of oxidative deamination, the following are selected as the preferred deuterated analogs of 2C-B:

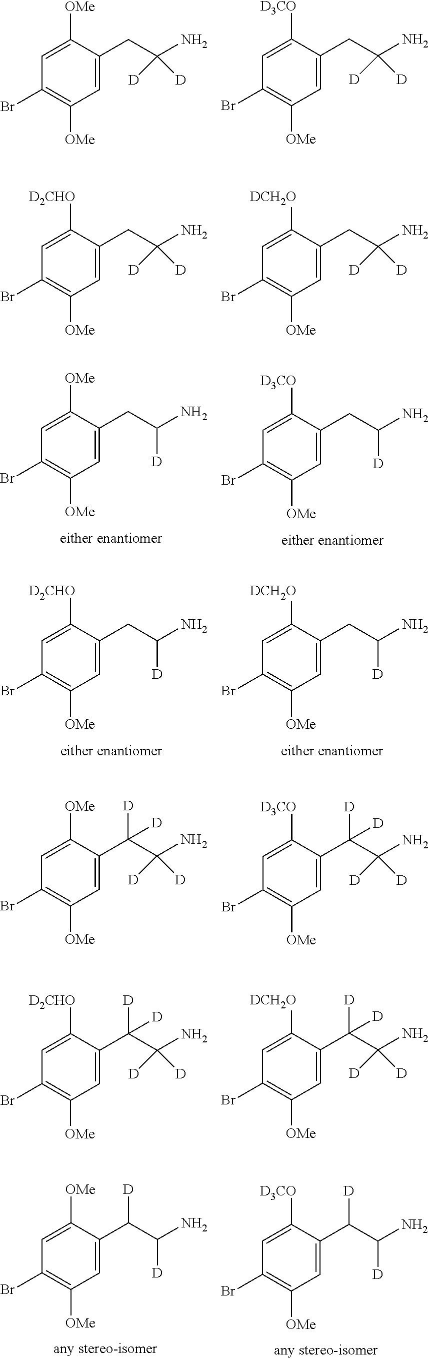

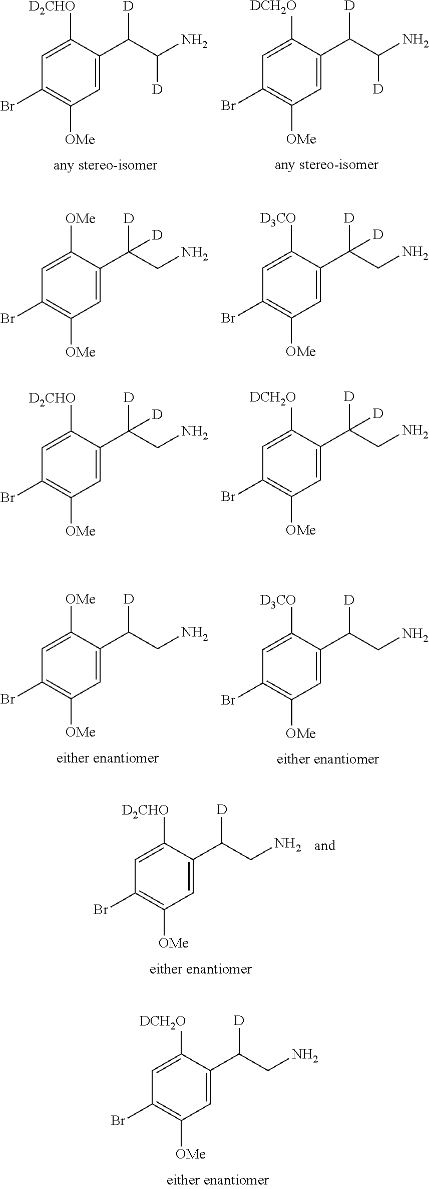

In some embodiments, Y₅ and Y₆ are each deuterium, and Y₁, Y₂, Y₃, and Y₄ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₅ and Y₆ are each deuterium, and Y₁, Y₂, Y₃, and Y₄ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₅ is deuterium, and Y₁, Y₂, Y₃, Y₄, and Y₆, are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₅ is deuterium, and Y₁, Y₂, Y₃, Y₄, and Y₆, are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃, Y₄, Y₅, and Y₆ are each deuterium, and Y₁ and Y₂ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₃, Y₄, Y₅, and Y₆ are each deuterium, and Y₁ and Y₂ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ and Y₆ are each deuterium, and Y₁, Y₂, Y₄, and Y₅ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₃ and Y₆ are each deuterium, and Y₁, Y₂, Y₄, and Y₅ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ and Y₄ are each deuterium, and Y₁, Y₂, Y₅, and Y₆ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₃ and Y₄ are each deuterium, and Y₁, Y₂, Y₅, and Y₆ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₃ is deuterium, and Y₁, Y₂, Y₄, Y₅, and Y₆ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₃ is deuterium, and Y₁, Y₂, Y₄, Y₅, and Y₆ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₃, Y₄, Y₅, and Y₆ are each deuterium. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, Y₁, Y₂, Y₃, Y₄, Y₅, and Y₆ are each deuterium. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₅, and Y₆ are each deuterium, and Y₃ and Y₄ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CH₃. In some embodiments, R₁ and R₂ are both CD₃. In some embodiments, R₁ is CD₃ and R₂ is CH₃. In some embodiments, R₁ is CH₃ and R₂ is CD₃.

In some embodiments, Y₁, Y₂, Y₅, and Y₆ are each deuterium, and Y₃ and Y₄ are each hydrogen. In one embodiment, R₁ is CD₃. In another embodiment, R₁ is CHD₂. In one embodiment, R₁ is CH₂D. In some embodiments, R₂ is CH₃.

In some embodiments, Y₁, Y₂, Y₃, Y₄, Y₅, and Y₆ are each hydrogen. In one embodiment, R₁ is CH₃. In another embodiment, R₁ is CHD₂. In one embodiment, R₁ is CH₂D. In one embodiment, R₁ is CD₃. In some embodiments, R₂ is CH₃. In another embodiment, R₂ is CHD₂. In one embodiment, R₂ is CH₂D. In some embodiments, R₂ is CD₃. In some embodiments, R₁ and R₂ are both CHD₂. In some embodiments, R₁ and R₂ are both CH₂D. In some embodiments, R₁ and R₂ are both CD₃. In some embodiments, R₁ is CD₃ and R₂ is CH₃. In some embodiments, R₁ is CH₃ and R₂ is CD₃. In some embodiments, R₁ is CH₃ and R₂ is CHD₂. In some embodiments, R₁ is CH₃ and R₂ is CH₂D. In some embodiments, R₁ is CHD₂ and R₂ is CH₃. In some embodiments, R₁ is CH₂D and R₂ is CH₃.

In some embodiments, the compound of Formula (II) has the formula (II-A):

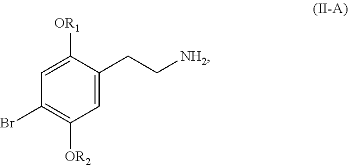

wherein at least one of R₁ and R₂ comprises one or more deuterium.

In some embodiments, the compound of Formula (II) has the formula (II-B):

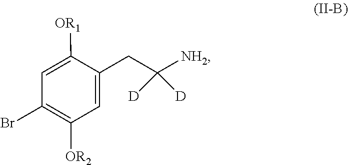

wherein R₁ and R₂ are each independently selected from CH₃, CH₂D, CHD₂, and CD₃.

In some embodiments, the compound of Formula (II) has the formula (IT-C):

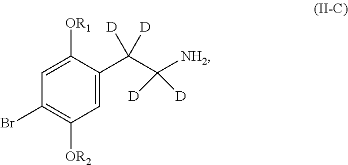

wherein R₁ and R₂ are each independently selected from CH₃, CH₂D, CHD₂, and CD₃.

### Deuteration of 2C-AL

In some embodiments, based on the site and degree of oxidative deamination, the following compound of Formula (IIm′) are selected as the preferred deuterated analogs of 2C-AL. In some embodiments, the compounds of Formula (IIm′) are selected from the group consisting or:

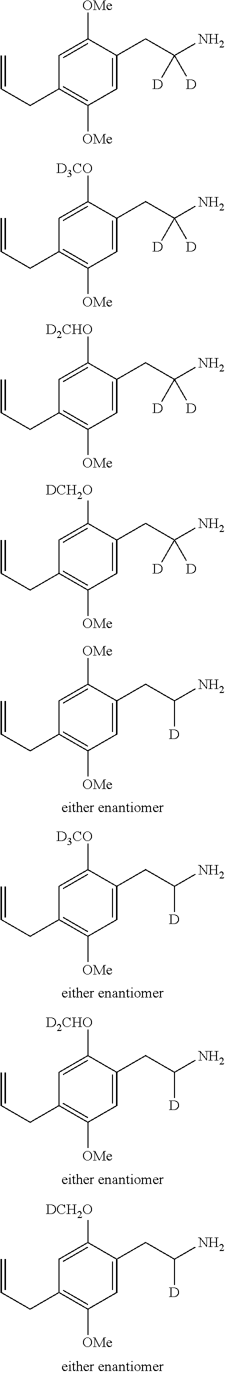

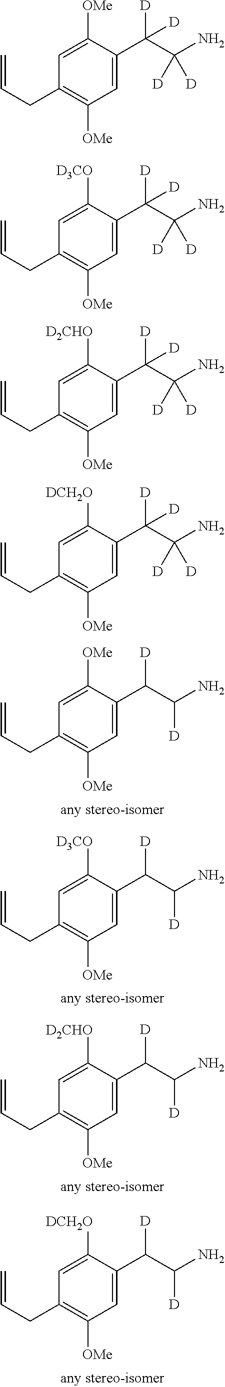

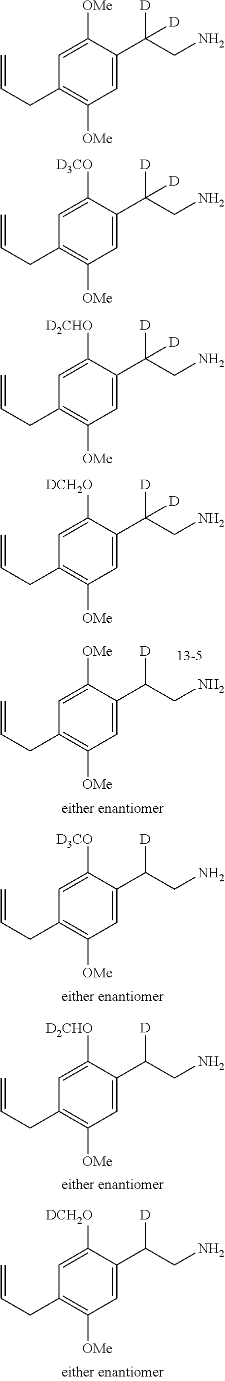

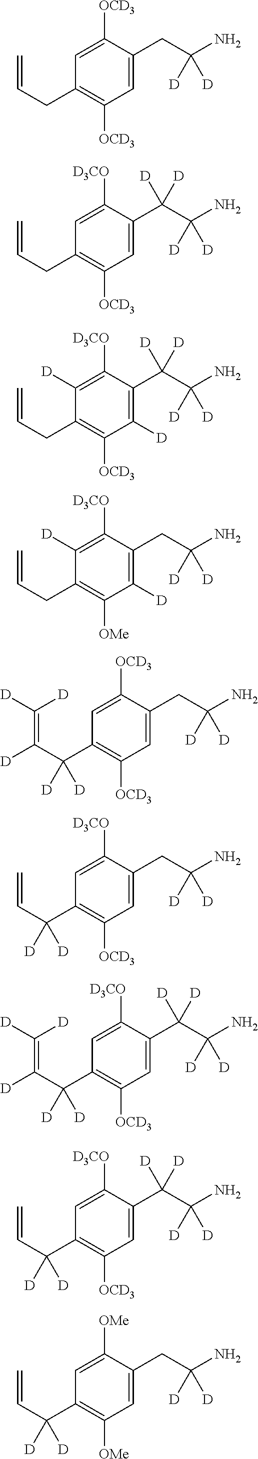

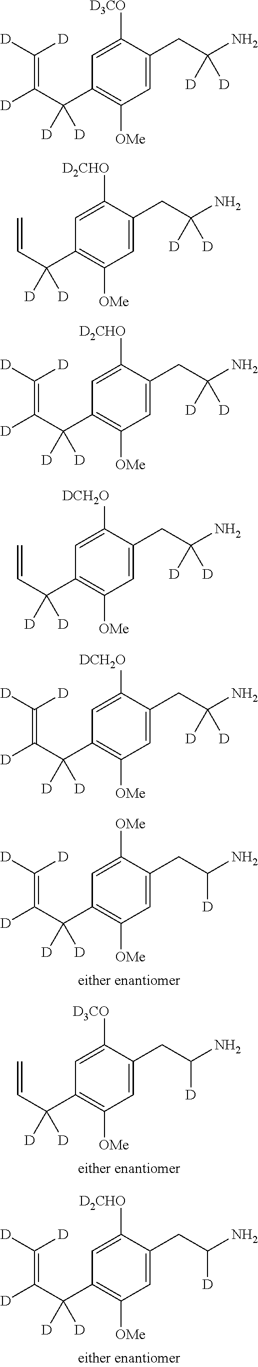

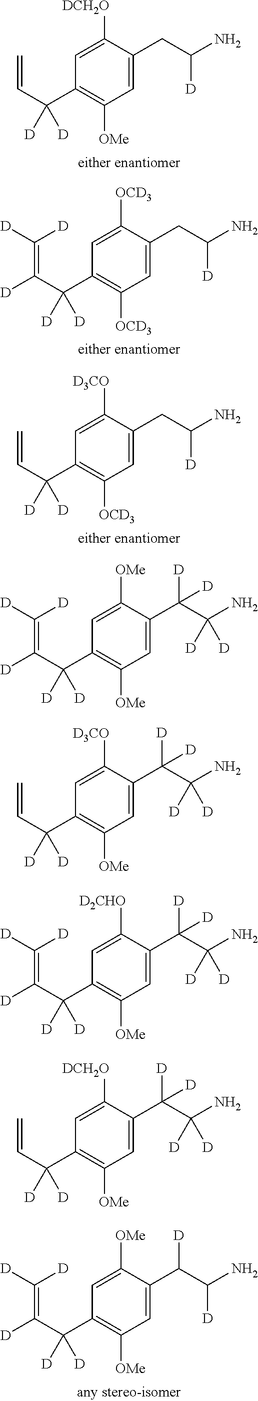

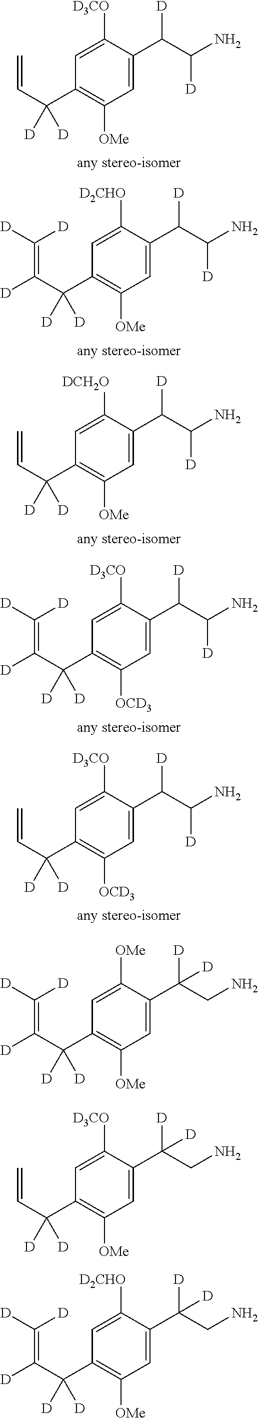

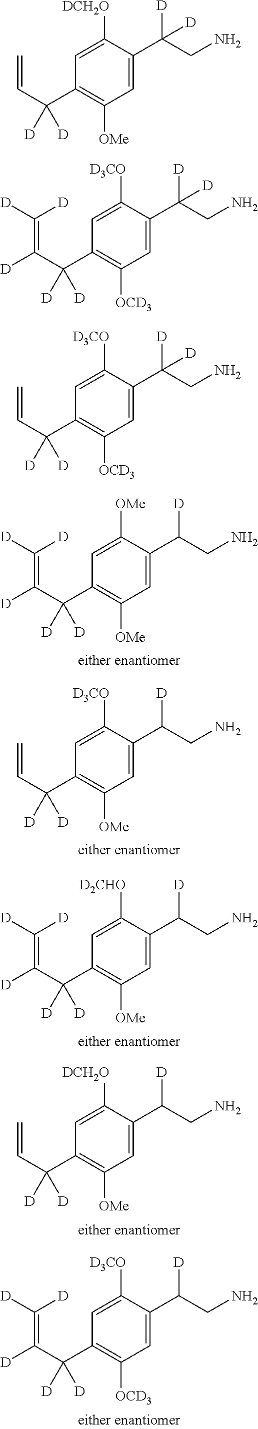

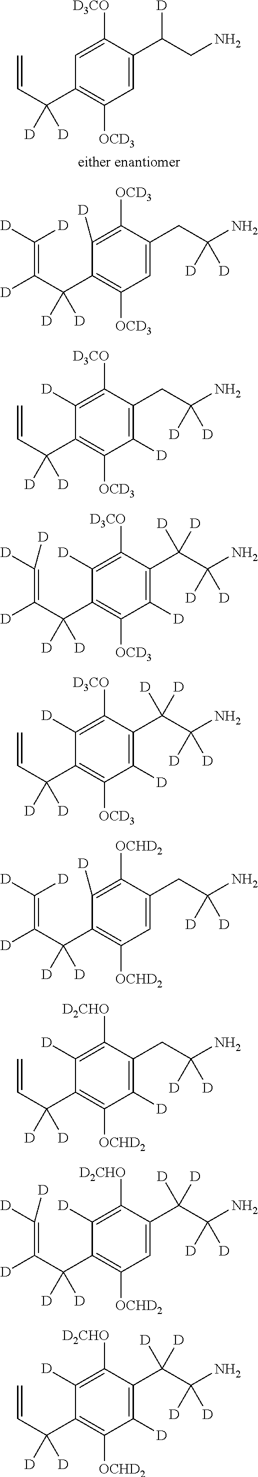

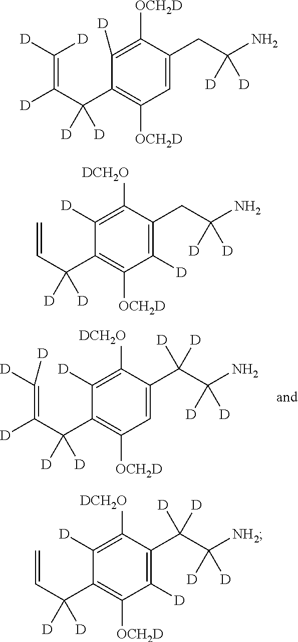

or a stereoisomer or pharmaceutically acceptable salt thereof.

Selected compounds of the disclosure with corresponding simplified molecular-input line-entry system (SMILES) strings are provided in Table 2.

In some embodiments, the compound of Formula (II) is a compound listed in Table 2.

TABLE 2

| | Structure |
|---|---|
| Cpd | SMILES |
| 1 | 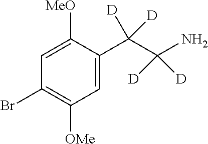 [SMILES: [2H]C([2H])(N)C([2H])([2H])c1cc(OC)c(Br)cc1OC] |
| | NC([2H])([2H])C([2H])([2H])C1═CC(O |
| | C)═C(Br)C═C1OC |
| | 2-(4-bromo-2,5- |
| | bis(methoxy)phenyl)ethan-1,1,2,2-d4-1- |
| | amine |
| 2 | 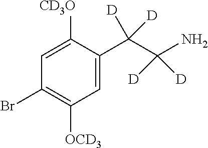 |
| | NC([2H])([2H])C([2H])([2H])C1═CC(O |
| | C([2H])([2H])[2H])═C(Br)C═C1OC([2H |
| | 1)([2H])[2H] |
| | 2-(4-bromo-2,5-bis(methoxy- |
| | d3)phenyl)ethan-1,1,2,2-d4-1-amine |
| 3 | 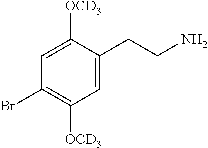 |
| | NCCC1═CC(OC([2H])([2H])[2H])═ |
| | C(Br)C═C1OC([2H])([2H])[2H] |
| | 2-(4-bromo-2,5-bis(methoxy- |
| | d3)phenyl)ethan-1-amine |
| 4 | 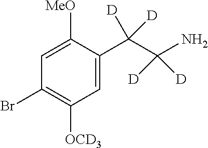 |
| | NC([2H])([2H])C([2H])([2H])C1═CC(O |
| | C([2H])([2H])[2H])═C(Br)C═C1OC |
| | 2-(4-bromo-2-methoxy-5-(methoxy- |
| | d₃)phenyl)ethan-1,1,2,2-d4-1-amine |
| 5 | 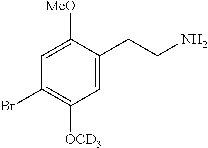 |
| | NCCC1═CC(OC([2H])([2H])[2H])═ |
| | C(Br)C═C1OC |
| | 2-(4-Bromo-2-methoxy-5-(methoxy- |
| | d₃)phenyl)ethan-1-amine |
| 6 | 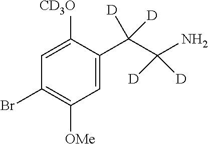 |
| | NC([2H])([2H])C([2H])([2H])C1═ |
| | CC(OC)═C(Br)C═C1OC([2H])([2H])[2H] |
| | 2-(4-bromo-5-methoxy-2-(methoxy- |
| | d₃)phenyl)ethan-1,1,2,2-d4-1-amine |
| 7 | 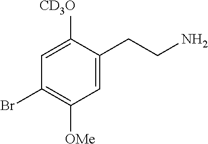 |
| | NCCC1═CC(OC)═C(Br)C═C1OC([2H]) |
| | ([2H])[2H] |
| | 2-(4-bromo-5-methoxy-2-(methoxy- |
| | d₃)phenyl)ethan-1-amine |
| 8 | 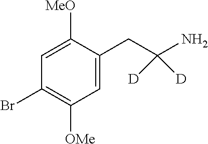 [SMILES: [2H]C([2H])(N)Cc1cc(OC)c(Br)cc1OC] |
| | NC([2H])([2H])CC1═CC(OC)═C(Br)C═ |
| | C1OC |
| | 2-(4-bromo-2,5- |
| | bis(methoxy)phenyl)ethan-1,1-d2-1- |
| | amine |
| 9 | 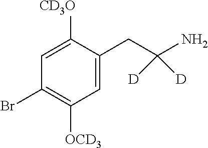 |
| | NC([2H])([2H])CC1═CC(OC([2H])([2H |
| | 1)[2H])═C(Br)C═C1OC([2H])([2H])[2H] |
| | 2-(4-bromo-2,5-bis(methoxy- |
| | d3)phenyl)ethan-1, 1-d2-1-amine |
| 10 | 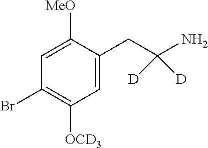 |
| | NC([2H])([2H])CC1═CC(OC([2H])([2H |
| | 1)[2H])═C(Br)C═C1OC |
| | 2-(4-bromo-2-methoxy-5-(methoxy- |
| | d₃)phenyl)ethan-1,1-d₂-1-amine |
| 11 | 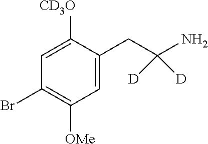 |
| | NC([2H])([2H])CC1═CC(OC)═C(Br)C═ |
| | C1OC([2H])([2H])[2H] |
| | 2-(4-bromo-5-methoxy-2-(methoxy- |
| | d₃)phenyl)ethan-1,1-d₂-1-amine |
| 12 | 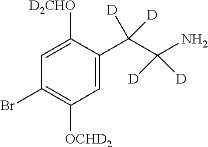 |
| | NC([2H])([2H])C([2H])([2H])C1═CC(O |
| | C([2H])[2H])═C(Br)C═C1OC([2H])[2H] |
| | 2-(4-bromo-2,5-bis(methoxy- |
| | d2)phenyl)ethan-1,1,2,2-d4-1-amine |
| 13 | 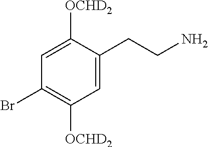 |
| | NCCC1═CC(OC([2H])[2H])═C(Br)C═ |
| | C1OC([2H])[2H] |
| | 2-(4-bromo-2,5-bis(methoxy- |
| | d2)phenyl)ethan-1-amine |
| 14 | 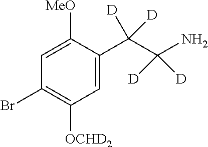 |
| | NC([2H])([2H])C([2H])([2H])C1═CC(O |
| | C([2H])[2H])═C(Br)C═C1OC |
| | 2-(4-bromo-2-methoxy-5-(methoxy- |
| | d₂)phenyl)ethan-1,1,2,2-d₄-1-amine |
| 15 | 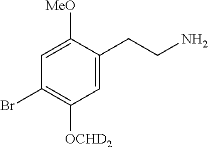 |
| | NCCC1═CC(OC([2H])[2H])═C(Br)C═ |
| | C1OC |
| | 2-(4-Bromo-2-methoxy-5-(methoxy- |
| | d₂)phenyl)ethan-1-amine |
| 16 | 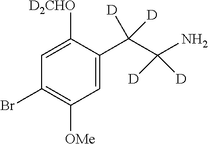 |
| | NC([2H])([2H])C([2H])([2H])C1═CC(O |
| | C)═C(Br)C═C1OC([2H])[2H] |
| | 2-(4-bromo-5-methoxy-2-(methoxy- |
| | d₂)phenyl)ethan-1,1,2,2-d₄-1-amine |
| 17 | 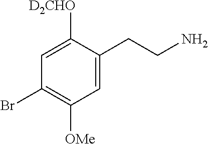 |
| | NCCC1═CC(OC)═C(Br)C═C1OC([2H]) |
| | [2H] |
| | 2-(4-bromo-5-methoxy-2-(methoxy- |
| | d₂)phenyl)ethan-1-amine |
| 18 | 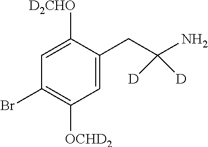 |
| | NC([2H])([2H])CC1═CC(OC([2H]) |
| | [2H])═C(Br)C═C1OC([2H])[2H] |
| | 2-(4-bromo-2,5-bis(methoxy- |
| | d2)phenyl)ethan-1,1-d2-1-amine |
| 19 | 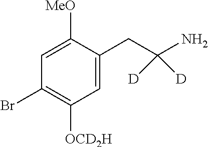 |
| | NC([2H])([2H])CC1═CC(OC([2H]) |
| | [2H])═C(Br)C═C1OC |
| | 2-(4-bromo-2-methoxy-5-(methoxy- |
| | d₂)phenyl)ethan-1,1-d₂-1-amine |
| 20 | 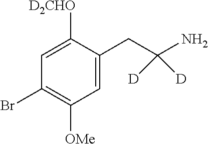 |
| | NC([2H])([2H])CC1═CC(OC)═C(Br)C═ |
| | C1OC([2H])[2H] |
| | 2-(4-bromo-5-methoxy-2-(methoxy- |
| | d₂)phenyl)ethan-1,1-d₂-1-amine |
| 21 | 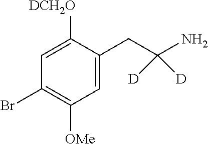 |
| | NC([2H])([2H])CC1═CC(OC)═C(Br)C═ |
| | C1OC[2H] |
| | 2-(4-bromo-5-methoxy-2-(methoxy- |
| | d₁)phenyl)ethan-1,1-d₂-1-amine |
| 22 | 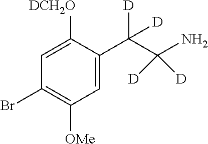 |
| | NC([2H])([2H])C([2H])([2H])C1═ |
| | CC(OC)═C(Br)C═C1OC[2H] |
| | 2-(4-bromo-5-methoxy-2-(methoxy- |
| | d₁)phenyl)ethan-1,1,2,2-d₄-1-amine |
| 23 | 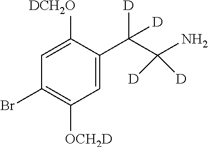 |
| | NC([2H])([2H])C([2H])([2H])C1═ |
| | CC(OC[2H])═C(Br)C═C1OC[2H] |
| | 2-(4-bromo-2,5-bis(methoxy- |
| | d₁)phenyl)ethan-1,1,2,2-d₄-1-amine |
| 24 | 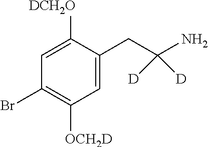 |
| | NC([2H])([2H])CC1═CC(OC[2H])═ |
| | C(Br)C═C1OC[2H] |
| | 2-(4-bromo-2,5-bis(methoxy- |
| | d₁)phenyl)ethan-1,1-d₂-1-amine |
| 25 | 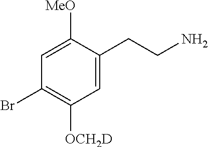 |
| | NCCC1=CC(OC[2H])═C(Br)C═C1OC |
| | 2-(4-bromo-2-methoxy-5-(methoxy- |
| | d₁)phenyl)ethan-1-amine |
| 26 | 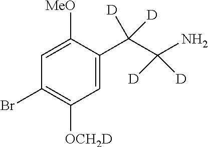 |
| | NC([2H])([2H])C([2H])([2H])C1═ |
| | CC(OC[2H])═C(Br)C═C1OC |
| | 2-(4-bromo-2-methoxy-5-(methoxy- |
| | d₁)phenyl)ethan-1,1,2,2-d₄-1-amine |
| 27 | 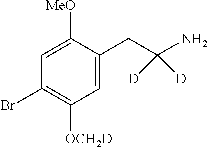 |
| | NC([2H])([2H])CC1═CC(OC[2H])═ |
| | C(Br)C═C1OC |
| | 2-(4-bromo-2-methoxy-5-(methoxy- |
| | d₁)phenyl)ethan-1,1-d₂-1-amine |

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutical Compositions

In another aspect, provided herein are pharmaceutical compositions of the compound described herein, such as a compound of Formula (I), (II), (IIa), (IIb), (IIc), (IId), (IIe), (IIf), (IIg), (IIh), (IIi), (IIj), (IIk), (IIl), (IIm), (IIm′), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), and a pharmaceutically acceptable carrier. In certain embodiments, the compound as described herein is administered as a pure chemical. In other embodiments, the compound described herein is combined with a pharmaceutically suitable or acceptable carrier (also referred to herein as a pharmaceutically acceptable excipient, physiologically acceptable excipient, or physiologically acceptable carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)), the disclosure of which is hereby incorporated herein by reference in its entirety.

In another aspect, provided herein are pharmaceutical compositions of the compound described herein, such as a compound of Formula (II), (II-A), (II-B), or (TI-C), and a pharmaceutically acceptable carrier. In certain embodiments, the compound as described herein is administered as a pure chemical. In other embodiments, the compound described herein is combined with a pharmaceutically suitable or acceptable carrier (also referred to herein as a pharmaceutically acceptable excipient, physiologically acceptable excipient, or physiologically acceptable carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)), the disclosure of which is hereby incorporated herein by reference in its entirety.

One embodiment provides a pharmaceutical composition comprising a pharmaceutically acceptable carrier and a compound of Formula (I), (II), (IIa), (IIb), (IIc), (IId), (IIe), (IIf), (IIg), (IIh), (IIi), (IIj), (IIk), (IIl), (IIm), (IIm′), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), or a pharmaceutically acceptable salt or solvate thereof.

Another embodiment provides a pharmaceutical composition comprising a pharmaceutically acceptable carrier and a compound of Formula (II), (II-A), (II-B), or (IT-C), or a pharmaceutically acceptable salt or solvate thereof

One embodiment provides a pharmaceutical composition comprising a pharmaceutically acceptable carrier and a compound of Formula (I), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), or a pharmaceutically acceptable salt or solvate thereof.

Another embodiment provides a pharmaceutical composition consisting essentially of a pharmaceutically acceptable carrier and a compound of Formula (II), (II-A), (II-B), or (II-C), or a pharmaceutically acceptable salt or solvate thereof.

Another embodiment provides a pharmaceutical composition consisting essentially of a pharmaceutically acceptable carrier and a compound of Formula (I), (II), (IIa), (IIb), (IIc), (IId), (IIe), (IIf), (IIg), (IIh), (IIi), (IIj), (IIk), (IIl), (IIm), (IIm′), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), or a pharmaceutically acceptable salt or solvate thereof.

Another embodiment provides a pharmaceutical composition consisting essentially of a pharmaceutically acceptable carrier and a compound of Formula (I), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (Ills), (IIIt), (IVa), or (IVb), or a pharmaceutically acceptable salt or solvate thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate, or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills, and capsules.

Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, the doses of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

“Pharmaceutically acceptable salt” includes both acid and base addition salts. A pharmaceutically acceptable salt of any one of the compounds described herein is intended to encompass any and all pharmaceutically suitable salt forms. Pharmaceutically acceptable salts of the compounds described herein are optionally pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). In some embodiments, acid addition salts of basic compounds are prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines, and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

### Methods of Use

In some embodiments, the compounds described herein, such as a compound of Formula (I), (II), (IIa), (IIb), (IIc), (IId), (IIe), (IIf), (IIg), (IIh), (Ili), (IIj), (IIk), (IIl), (IIm), (IIm′), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), can be used for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, the compounds described herein, such as a compound of Formula (II), (II-A), (II-B), or (IT-C), can be used for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, the compounds described herein, such as a compound of Formula (I), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IVa), or (IVb), can be used for increasing neuronal plasticity in a subject.

In some embodiments, the compounds described herein, such as a compound of Formula (II), (II-A), (II-B), or (IT-C), can be used for increasing neuronal plasticity in a subject.

In some embodiments, the compounds described herein can also be used to treat any brain disease or disorder. In some embodiments, the compounds described herein can also be used for increasing at least one of translation, transcription or secretion of neurotrophic factors. In some embodiments, the compound has, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the brain disease or disorder is a neurodegenerative disorder, Alzheimer's disease or Parkinson's disease. In some embodiments, the brain disease or disorder is psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, a compound described herein is used to treat a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, increasing neuronal plasticity by treating a subject with a compound the present disclosure can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, a compound disclosed herein is used to increase neuronal plasticity and has, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). Brain disorders can include, for example, migraines, addiction (e.g., substance use disorder), depression, and anxiety.

In some embodiments, the experiment or assay to determine increased neuronal plasticity derived from the administration of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the condition is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

As used herein, “treatment” or “treating” or “palliating” or “ameliorating” are used interchangeably herein. These terms refer to an approach for obtaining beneficial or desired results including, but not limited to, therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

Provided herein are methods of treat neurological diseases in a subject need thereof comprising administering a compound described herein (e.g., a compound of Formula (I) such as a compound of Formula (II)) to the subject. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In another aspect, provided herein are methods of treating a disease or disorder, comprising administering to a subject in need thereof a compound described herein (e.g., a compound of Formula (I)). In some embodiments, a therapeutically effective amount of the compound of Formula (I) is administered. In some embodiments, the disease or disorder is a musculoskeletal pain disorder including fibromyalgia, muscle pain, joint stiffness, osteoarthritis, rheumatoid arthritis, muscle cramps. In some embodiments, provided herein are method of treating a disease of women's reproductive health, for example, premenstrual dysphoric disorder (PMDD), premenstrual syndrome (PMS), post-partum depression, and menopause.

In some embodiments, the compounds described herein (e.g., a compound of Formula (I) such as a compound of Formula (II)) have activity as 5-HT₂A modulators. In some embodiments, the compounds described herein (e.g., a compound of Formula (I) such as a compound of Formula (II)) elicit a biological response by activating the 5-HT₂-A receptor (e.g., allosteric modulation or modulation of a biological target that activates the 5-HT₂-A receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT₂-A agonist activity, for example, DMT, LSD, and DOI. In some embodiments, the compounds described herein (e.g., a compound of Formula (I)) are 5-HT₂-A modulators and promote neural plasticity (e.g., cortical structural plasticity). In some embodiments, the compounds described herein (e.g., a compound of Formula (I)) are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT₂-A modulators (e.g., 5-HT₂-A agonists) are non-hallucinogenic. In some embodiments, non-hallucinogenic 5-HT₂-A modulators (e.g., 5-HT₂-A agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

### Methods of Treating a Brain Disorder

In yet another aspect, provided herein are method for treating a brain disorder in a subject in need thereof, comprising administering the compounds described herein (e.g., a compound of Formula (I)) to the subject. The compounds described herein (e.g., a compound of Formula (I) such as a compound of Formula (II)) can function as 5-HT₂-A agonists alone, or in combination with a second therapeutic agent that also is a 5-HT₂-A modulator. In such cases the second therapeutic agent can be an agonist or an antagonist. In some embodiments, administering a 5-HT₂-A antagonist in combination with a compound of the present invention to mitigate undesirable effects of 5-HT₂-A agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, Sβ242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, or lumateperone, or a pharmaceutically acceptable salt, solvate, metabolite, deuterated analogue, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about hours prior administration the compounds described herein (e.g., a compound of Formula (I) such as a compound of Formula (II)). In some embodiments, the serotonin receptor modulator is administered at most about one hour prior to the compounds described herein (e.g., a compound of Formula (I) such as a compound of Formula (II)). Thus, in some embodiments of combination therapy with the compounds described herein (e.g., a compound of Formula (I) such as a compound of Formula (II)), the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain embodiments, the compound described herein (e.g., a compound of Formula (I) such as a compound of Formula (II)) is provided at a dose of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg and the serotonin receptor modulator is provided at a dose of about 10 mg to about 100 mg.

In some embodiments, the compounds described herein (e.g., a compound of Formula (I) such as a compound of Formula (II)) are non-hallucinogenic 5-HT_{2A} modulators (e.g., 5-HT_{2A} agonists) and are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, the compounds described herein (e.g., a compound of Formula (I) such as a compound of Formula (II)) are non-hallucinogenic 5-HT_{2A} modulators (e.g., 5-HT_{2A} agonists) and are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-HT_{2A} agonists) are used for increasing at least one of translation, transcription, or secretion of neurotrophic factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the compounds described herein (e.g., a compound of Formula (I) such as a compound of Formula (II)) are administered at a low dose that is lower than a dose that would produce noticeable psychedelic effects but high enough to provide a therapeutic benefit. This dose range is predicted to be between 200 μg (micrograms) and 2 mg.

In some embodiments, oral doses typically range from about 1.0 mg to about 350 mg, one to four times, or more, per day. In certain embodiments, the compounds are administered to a subject at a daily dosage of between 0.01 mg/kg to about 50 mg/kg of body weight. In other embodiments, the dose is from 1 to 350 mg/day. In certain embodiments, the daily dose is from 1 to 750 mg/day; or from 10 to 350 mg/day. In certain embodiments, the compounds are provided at a daily dosage of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg.

In some embodiments, the compounds disclosed herein (e.g., a compound of Formula (II)), including those described in Table 2, is provided at a daily dose of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg.

In some embodiments, 2-(4-bromo-5-methoxy-2-(methoxy-d3)phenyl)ethan-1-amine hydrochloride is provided at a daily dose of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg.

In some embodiments, Compound 2 described in Table 2 is provided at a daily dose of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg.

In particular embodiments of treating the disorders described above, combination therapy is used as described herein. In one embodiment of such therapy a compound described herein (e.g., a compound of Formula (I) such as a compound of Formula (II)) is administered in combination with a serotonin receptor modulator. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, or lumateperone, or a pharmaceutically acceptable salt, solvate, metabolite, deuterated analogue, derivative, prodrug, or combinations thereof.

In some embodiments, the serotonin receptor modulator for combination with the presently disclosed compounds is selected from glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin (ACO-103), nelotanserin, lorcaserin, flibanserin, roluperiodone or a pharmaceutically acceptable salt, solvate, metabolite, deuterated analogue, derivative, prodrug, or combinations thereof.

In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the serotonin receptor modulator is ketanserin or a pharmaceutically acceptable salt, solvate, metabolite, deuterated analogue, derivative, or prodrug thereof. In some embodiments, the serotonin receptor modulator is pimavanserin or a pharmaceutically acceptable salt, solvate, metabolite, deuterated analogue, derivative, or prodrug thereof. In some embodiments, the serotonin receptor modulator is eplivanserin or a pharmaceutically acceptable salt, solvate, metabolite, deuterated analogue, derivative, or prodrug thereof. In some embodiments, the serotonin receptor modulator is flibanserin or a pharmaceutically acceptable salt, solvate, metabolite, deuterated analogue, derivative, or prodrug thereof. In some embodiments, the serotonin receptor modulator is roluperiodone or a pharmaceutically acceptable salt, solvate, metabolite, deuterated analogue, derivative, or prodrug thereof.

In some embodiments, the serotonin receptor modulator is administered prior to a compound disclosed herein, such as from about one to about three hours prior to administration of a compound disclosed herein. In some embodiments, the serotonin receptor modulator is administered at most about one hour prior to the presently disclosed compound.

In some embodiments, the serotonin receptor modulator for use with the compounds disclosed herein (e.g., a compound of Formula (II)), including those described in Table 2, is eplivanserin, wherein the eplivanserin is administered in about 1 mg to about 40 mg, or about 5 mg to about 10 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is volinanserin, wherein the volinanserin is administered in about 1 mg to about 60 mg, or about 5 mg to about 20 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is ketanserin, wherein the ketanserin is administered in about 10 mg to about 80 mg, about 30 mg to about 50 mg, or about 40 mg and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is ritanserin, wherein the ritanserin is administered in about 1 mg to about 40 mg, or about 2.5 mg to about 10 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is pimavanserin, wherein the pimavanserin is administered in about 1 mg to about 60 mg, or about 17 mg to about 34 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is nelotanserin, wherein the nelotanserin is administered in about 1 mg to about 80 mg, or about 40 mg to about 80 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is pruvanserin, wherein the pruvanserin is administered in about 1 mg to about 40 mg, or about 3 mg to about 10 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is flibanserin, wherein the flibanserin is administered in about 10 mg to about 200 mg, or about 80 mg to about 120 mg, or about 100 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is olanzapine, wherein the olanzapine is administered in about 2.5 mg to about 30 mg, or about 5 mg or about 10 mg, or about 20 mg or about 25 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is quetiapine, wherein the quetiapine is administered in about 25 mg to about 800 mg, or about 50 mg to about 100 mg, or about 150 mg or about 200 mg or about 250 mg or about 300 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is an extended-release of quetiapine, wherein the extended-release of quetiapine is administered in about 50 mg to about 300 mg, or about 50 mg or about 100 mg or about 200 mg, or about 300 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the compound disclosed herein (e.g., a compound of Formula (II)), including those described in Table 2, is administered between about 1 mg and 50 mg.

In some embodiments, the serotonin receptor modulator for use with the compounds disclosed herein (e.g., a compound of Formula (II)), including those described in Table 2, is an extended-release of risperidone including (RISPERDAL CONSTA), wherein the extended-release of risperidone is administered in about 12.5 mg, or about 25 mg, or about 37.5 mg, or about 50 mg, and the compound disclosed herein (e.g., a compound of Formula (II)), including those described in Table 2, is administered between about 1 mg and 50 mg.

In certain embodiments, such as those described above a compound disclosed herein (e.g., a compound of Formula (II)), including those described in Table 2, is co-administered with a serotonin receptor modulator in the same or in separate compositions. In one embodiment, the serotonin receptor modulator is administered prior to the compound disclosed herein (e.g., a compound of Formula (II)), including those described in Table 2. In one embodiment, the compound disclosed herein (e.g., a compound of Formula (II)), including those described in Table 2, is administered in a modified release formulation such that the subject is effectively pretreated with serotonin receptor modulator prior to release of an effective amount of the compound. In some embodiments the serotonin receptor modulator is part of a single fixed dose formulation that releases serotonin receptor modulator first followed by the compound on two different release profiles. In another embodiment the serotonin receptor modulator is administered first as a single dosage and after a length of time, the compound disclosed herein (e.g., a compound of Formula (II)), including those described in Table 2, is administered as a second dosage separate from the first dosage.

In some embodiments, the serotonin receptor modulator is eplivanserin, wherein the eplivanserin is administered to pretreat at least 15 minutes prior to the administration of the compound disclosed herein (e.g., a compound of Formula (II)), including those described in Table 2. In some embodiments, the serotonin receptor modulator is eplivanserin, wherein the eplivanserin is administered to pretreat between at least 30 minutes prior and 360 minutes prior to the release or administration of the compound disclosed herein (e.g., a compound of Formula (II)), including those described in Table 2. In some embodiments, the serotonin receptor modulator is eplivanserin, wherein the eplivanserin is administered to pretreat between at least 60 minutes prior and 360 minutes prior to the release or administration of the compound disclosed herein (e.g., a compound of Formula (II)), including those described in Table 2. In some embodiments, the serotonin receptor modulator is eplivanserin, wherein the eplivanserin is administered to pretreat at between least 90 minutes and 240 minutes prior to the compound disclosed herein (e.g., a compound of Formula (II)), including those described in Table 2. In some embodiments, the serotonin receptor modulator is eplivanserin, wherein the eplivanserin is administered to pretreat at least 120 minutes prior to the compound disclosed herein (e.g., a compound of Formula (II)), including those described in Table 2.

In some embodiments, the serotonin receptor modulator is eplivanserin, wherein the eplivanserin is administered to pretreat at least 150 minutes prior to the compound disclosed herein (e.g., a compound of Formula (II)), including those described in Table 2. In some embodiments, the serotonin receptor modulator is eplivanserin, wherein the eplivanserin is administered to pretreat at least 180 minutes prior to the compound disclosed herein (e.g., a compound of Formula (II)), including those described in Table 2. In some embodiments, the serotonin receptor modulator is eplivanserin, wherein the eplivanserin is administered to pretreat at least 210 minutes prior to the compound disclosed herein (e.g., a compound of Formula (II)), including those described in Table 2. In some embodiments, the serotonin receptor modulator is eplivanserin, wherein the eplivanserin is administered to pretreat at least 240 minutes prior to the compound disclosed herein (e.g., a compound of Formula (II)), including those described in Table 2.

In some embodiments, the serotonin receptor modulator is eplivanserin, wherein the eplivanserin is administered to pretreat at least 270 minutes prior to the compound disclosed herein (e.g., a compound of Formula (II)), including those described in Table 2. In some embodiments, the serotonin receptor modulator is eplivanserin, wherein the eplivanserin is administered to pretreat at least 300 minutes prior to the compound disclosed herein (e.g., a compound of Formula (II)), including those described in Table 2. In some embodiments, the serotonin receptor modulator is eplivanserin, wherein the eplivanserin is administered to pretreat at least 330 minutes prior to the compound disclosed herein (e.g., a compound of Formula (II)), including those described in Table 2. In some embodiments, the serotonin receptor modulator is eplivanserin, wherein the eplivanserin is administered to pretreat at least 360 minutes prior to the compound disclosed herein (e.g., a compound of Formula (II)), including those described in Table 2.

In some preferred embodiments, the serotonin receptor modulator is eplivanserin, wherein eplivanserin is administered to pretreat between about 60 minutes and about 180 minutes prior to the administration of the compound disclosed herein (e.g., a compound of Formula (II)), including those described in Table 2.

In some embodiments, the serotonin receptor modulator is volinanserin, wherein the volinanserin is administered to pretreat a subject between at least 15 minutes and 360 minutes prior to the administration or release of the compound disclosed herein (e.g., a compound of Formula (II)), including those described in Table 2. In some embodiments, the serotonin receptor modulator is volinanserin, wherein the volinanserin is administered to pretreat between at least 30 minutes and 360 minutes prior to the administration or release of the compound disclosed herein (e.g., a compound of Formula (II)), including those described in Table 2. In some embodiments, the serotonin receptor modulator is volinanserin, wherein the volinanserin is administered to pretreat between at least 60 minutes and 240 minutes prior to the administration or release of the compound disclosed herein (e.g., a compound of Formula (II)), including those described in Table 2. In some embodiments, the serotonin receptor modulator is volinanserin, wherein the volinanserin is administered to pretreat at least 90 minutes prior to the compound disclosed herein (e.g., a compound of Formula (II)), including those described in Table 2. In some embodiments, the serotonin receptor modulator is volinanserin, wherein the volinanserin is administered to pretreat at least 120 minutes prior to the compound disclosed herein (e.g., a compound of Formula (II)), including those described in Table 2.

In some embodiments, the serotonin receptor modulator is volinanserin, wherein the volinanserin is administered to pretreat at least 150 minutes prior to the compound disclosed herein (e.g., a compound of Formula (II)), including those described in Table 2. In some embodiments, the serotonin receptor modulator is volinanserin, wherein the volinanserin is administered to pretreat between about 15 minutes and about 150 minutes prior to the compound disclosed herein (e.g., a compound of Formula (II)), including those described in Table 2. In some embodiments, the serotonin receptor modulator is volinanserin, wherein the volinanserin is administered to pretreat at least 180 minutes prior to the compound disclosed herein (e.g., a compound of Formula (II)), including those described in Table 2. In some embodiments, the serotonin receptor modulator is volinanserin, wherein the volinanserin is administered to pretreat at least 210 minutes prior to the compound disclosed herein (e.g., a compound of Formula (II)), including those described in Table 2. In some embodiments, the serotonin receptor modulator is volinanserin, wherein the volinanserin is administered to pretreat at least 240 minutes prior to the compound disclosed herein (e.g., a compound of Formula (II)), including those described in Table 2. In some embodiments, the serotonin receptor modulator is volinanserin, wherein the volinanserin is administered to pretreat at least 270 minutes prior to the compound disclosed herein (e.g., a compound of Formula (II)), including those described in Table 2. In some embodiments, the serotonin receptor modulator is volinanserin, wherein the volinanserin is administered to pretreat at least 300 minutes prior to the compound disclosed herein (e.g., a compound of Formula (II)), including those described in Table 2. In some embodiments, the serotonin receptor modulator is volinanserin, wherein the volinanserin is administered to pretreat at least 330 minutes prior to the compound disclosed herein (e.g., a compound of Formula (II)), including those described in Table 2. In some embodiments, the serotonin receptor modulator is volinanserin, wherein the volinanserin is administered to pretreat at least 360 minutes prior to the compound disclosed herein (e.g., a compound of Formula (II)), including those described in Table 2. In some preferred embodiments, the serotonin receptor modulator is volinanserin, wherein volinanserin is administered to pretreat between about 60 minutes and about 180 minutes prior to the administration of the compound disclosed herein (e.g., a compound of Formula (II)), including those described in Table 2.

In some embodiments, the serotonin receptor modulator is ketanserin, wherein the ketanserin is administered to pretreat at least 15 minutes prior to the administration of the compound disclosed herein (e.g., a compound of Formula (II)), including those described in Table 2. In some embodiments, the serotonin receptor modulator is ketanserin, wherein the ketanserin is administered to pretreat between at least 30 minutes and 360 minutes prior to the administration or release of the compound disclosed herein (e.g., a compound of Formula (II)), including those described in Table 2. In some embodiments, the serotonin receptor modulator is ketanserin, wherein the ketanserin is administered to pretreat between at least 60 minutes and 240 minutes prior to the administration or release of the compound disclosed herein (e.g., a compound of Formula (II)), including those described in Table 2. In some embodiments, the serotonin receptor modulator is ketanserin, wherein the ketanserin is administered to pretreat at least 90 minutes prior to the compound disclosed herein (e.g., a compound of Formula (II)), including those described in Table 2. In some embodiments, the serotonin receptor modulator is ketanserin, wherein the ketanserin is administered to pretreat at least 120 minutes prior to the compound disclosed herein (e.g., a compound of Formula (II)), including those described in Table 2. In some embodiments, the serotonin receptor modulator is ketanserin, wherein the ketanserin is administered to pretreat at least 150 minutes prior to the compound disclosed herein (e.g., a compound of Formula (II)), including those described in Table 2. In some embodiments, the serotonin receptor modulator is ketanserin, wherein the ketanserin is administered to pretreat between about 15 minutes and about 150 minutes prior to the compound disclosed herein (e.g., a compound of Formula (II)), including those described in Table 2. In some embodiments, the serotonin receptor modulator is ketanserin, wherein the ketanserin is administered to pretreat at least 180 minutes prior to the compound disclosed herein (e.g., a compound of Formula (II)), including those described in Table 2. In some embodiments, the serotonin receptor modulator is ketanserin, wherein the ketanserin is administered to pretreat at least 210 minutes prior to the compound disclosed herein (e.g., a compound of Formula (II)), including those described in Table 2. In some embodiments, the serotonin receptor modulator is ketanserin, wherein the ketanserin is administered to pretreat at least 240 minutes prior to the compound disclosed herein (e.g., a compound of Formula (II)), including those described in Table 2. In some embodiments, the serotonin receptor modulator is ketanserin, wherein the ketanserin is administered to pretreat at least 270 minutes prior to the compound disclosed herein (e.g., a compound of Formula (II)), including those described in Table 2. In some embodiments, the serotonin receptor modulator is ketanserin, wherein the ketanserin is administered to pretreat at least 300 minutes prior to the compound disclosed herein (e.g., a compound of Formula (II)), including those described in Table 2. In some embodiments, the serotonin receptor modulator is ketanserin, wherein the ketanserin is administered to pretreat at least 330 minutes prior to the compound disclosed herein (e.g., a compound of Formula (II)), including those described in Table 2. In some embodiments, the serotonin receptor modulator is ketanserin, wherein the ketanserin is administered to pretreat at least 360 minutes prior to the compound disclosed herein (e.g., a compound of Formula (II)), including those described in Table 2. In some preferred embodiments, the serotonin receptor modulator is ketanserin, wherein ketanserin is administered to pretreat between about 60 minutes and about 180 minutes prior to the administration of the compound disclosed herein (e.g., a compound of Formula (II)), including those described in Table 2.

In some embodiments, the serotonin receptor modulator is ritanserin, wherein the ritanserin is administered to pretreat at least 15 minutes prior to the administration of the compound disclosed herein (e.g., a compound of Formula (II)), including those described in Table 2. In some embodiments, the serotonin receptor modulator is ritanserin, wherein the ritanserin is administered to pretreat at least 30 minutes prior to the compound disclosed herein (e.g., a compound of Formula (II)), including those described in Table 2. In some embodiments, the serotonin receptor modulator is ritanserin, wherein the ritanserin is administered to pretreat between at least 60 minutes and 240 minutes prior to the administration or release of the compound disclosed herein (e.g., a compound of Formula (II)), including those described in Table 2. In some embodiments, the serotonin receptor modulator is ritanserin, wherein the ritanserin is administered to pretreat at least 90 minutes prior to the compound disclosed herein (e.g., a compound of Formula (II)), including those described in Table 2. In some embodiments, the serotonin receptor modulator is ritanserin, wherein the ritanserin is administered to pretreat at least 120 minutes prior to the compound disclosed herein (e.g., a compound of Formula (II)), including those described in Table 2. In some embodiments, the serotonin receptor modulator is ritanserin, wherein the ritanserin is administered to pretreat at least 150 minutes prior to the compound disclosed herein (e.g., a compound of Formula (II)), including those described in Table 2. In some embodiments, the serotonin receptor modulator is ritanserin, wherein the ritanserin is administered to pretreat between about 15 minutes and about 150 minutes prior to the compound disclosed herein (e.g., a compound of Formula (II)), including those described in Table 2. In some embodiments, the serotonin receptor modulator is ritanserin, wherein the ritanserin is administered to pretreat at least 180 minutes prior to the compound disclosed herein (e.g., a compound of Formula (II)), including those described in Table 2. In some embodiments, the serotonin receptor modulator is ritanserin, wherein the ritanserin is administered to pretreat at least 210 minutes prior to the compound disclosed herein (e.g., a compound of Formula (II)), including those described in Table 2. In some embodiments, the serotonin receptor modulator is ritanserin, wherein the ritanserin is administered to pretreat at least 240 minutes prior to the compound disclosed herein (e.g., a compound of Formula (II)), including those described in Table 2. In some embodiments, the serotonin receptor modulator is ritanserin, wherein the ritanserin is administered to pretreat at least 270 minutes prior to the compound disclosed herein (e.g., a compound of Formula (II)), including those described in Table 2. In some embodiments, the serotonin receptor modulator is ritanserin, wherein the ritanserin is administered to pretreat at least 300 minutes prior to the compound disclosed herein (e.g., a compound of Formula (II)), including those described in Table 2. In some embodiments, the serotonin receptor modulator is ritanserin, wherein the ritanserin is administered to pretreat at least 330 minutes prior to the compound disclosed herein (e.g., a compound of Formula (II)), including those described in Table 2. In some embodiments, the serotonin receptor modulator is ritanserin, wherein the ritanserin is administered to pretreat at least 360 minutes prior to the compound disclosed herein (e.g., a compound of Formula (II)), including those described in Table 2. In some preferred embodiments, the serotonin receptor modulator is ritanserin, wherein ritanserin is administered to pretreat between about 60 minutes and about 180 minutes prior to the administration of the compound disclosed herein (e.g., a compound of Formula (II)), including those described in Table 2.

In some embodiments, the serotonin receptor modulator is pimavanserin, wherein the pimavanserin is administered to pretreat at least 15 minutes prior to the administration of the compound disclosed herein (e.g., a compound of Formula (II)), including those described in Table 2. In some embodiments, the serotonin receptor modulator is pimavanserin, wherein the pimavanserin is administered to pretreat at least 30 minutes prior to the compound disclosed herein (e.g., a compound of Formula (II)), including those described in Table 2. In some embodiments, the serotonin receptor modulator is pimavanserin, wherein the pimavanserin is administered to pretreat between at least 60 minutes and 240 minutes prior to the administration or release of the compound disclosed herein (e.g., a compound of Formula (II)), including those described in Table 2. In some embodiments, the serotonin receptor modulator is pimavanserin, wherein the pimavanserin is administered to pretreat at least 90 minutes prior to the compound disclosed herein (e.g., a compound of Formula (II)), including those described in Table 2. In some embodiments, the serotonin receptor modulator is pimavanserin, wherein the pimavanserin is administered to pretreat at least 120 minutes prior to the compound disclosed herein (e.g., a compound of Formula (II)), including those described in Table 2. In some embodiments, the serotonin receptor modulator is pimavanserin, wherein the pimavanserin is administered to pretreat at least 150 minutes prior to the compound disclosed herein (e.g., a compound of Formula (II)), including those described in Table 2. In some embodiments, the serotonin receptor modulator is pimavanserin, wherein the pimavanserin is administered to pretreat between about 15 minutes and about 150 minutes prior to the compound disclosed herein (e.g., a compound of Formula (II)), including those described in Table 2. In some embodiments, the serotonin receptor modulator is pimavanserin, wherein the pimavanserin is administered to pretreat at least 180 minutes prior to the compound disclosed herein (e.g., a compound of Formula (II)), including those described in Table 2. In some embodiments, the serotonin receptor modulator is pimavanserin, wherein the pimavanserin is administered to pretreat at least 210 minutes prior to the compound disclosed herein (e.g., a compound of Formula (II)), including those described in Table 2. In some embodiments, the serotonin receptor modulator is pimavanserin, wherein the pimavanserin is administered to pretreat at least 240 minutes prior to the compound disclosed herein (e.g., a compound of Formula (II)), including those described in Table 2. In some embodiments, the serotonin receptor modulator is pimavanserin, wherein the pimavanserin is administered to pretreat at least 270 minutes prior to the compound disclosed herein (e.g., a compound of Formula (II)), including those described in Table 2. In some embodiments, the serotonin receptor modulator is pimavanserin, wherein the pimavanserin is administered to pretreat at least 300 minutes prior to the compound disclosed herein (e.g., a compound of Formula (II)), including those described in Table 2. In some embodiments, the serotonin receptor modulator is pimavanserin, wherein the pimavanserin is administered to pretreat at least 330 minutes prior to the compound disclosed herein (e.g., a compound of Formula (II)), including those described in Table 2. In some embodiments, the serotonin receptor modulator is pimavanserin, wherein the pimavanserin is administered to pretreat at least 360 minutes prior to the compound disclosed herein (e.g., a compound of Formula (II)), including those described in Table 2. In some preferred embodiments, the serotonin receptor modulator is pimavanserin, wherein pimavanserin is administered to pretreat between about 60 minutes and about 180 minutes prior to the administration of the compound disclosed herein (e.g., a compound of Formula (II)), including those described in Table 2.

In some embodiments, the serotonin receptor modulator is nelotanserin, wherein the nelotanserin is administered to pretreat at least 15 minutes prior to the administration of the compound disclosed herein (e.g., a compound of Formula (II)), including those described in Table 2. In some embodiments, the serotonin receptor modulator is nelotanserin, wherein the nelotanserin is administered to pretreat at least 30 minutes prior to the compound disclosed herein (e.g., a compound of Formula (II)), including those described in Table 2. In some embodiments, the serotonin receptor modulator is nelotanserin, wherein the nelotanserin is administered to pretreat between at least 60 minutes and 240 minutes prior to the administration or release of the compound disclosed herein (e.g., a compound of Formula (II)), including those described in Table 2. In some embodiments, the serotonin receptor modulator is nelotanserin, wherein the nelotanserin is administered to pretreat at least 90 minutes prior to the compound disclosed herein (e.g., a compound of Formula (II)), including those described in Table 2. In some embodiments, the serotonin receptor modulator is nelotanserin, wherein the nelotanserin is administered to pretreat at least 120 minutes prior to the compound disclosed herein (e.g., a compound of Formula (II)), including those described in Table 2.

In some embodiments, the serotonin receptor modulator is nelotanserin, wherein the nelotanserin is administered to pretreat at least 150 minutes prior to the compound disclosed herein (e.g., a compound of Formula (II)), including those described in Table 2. In some embodiments, the serotonin receptor modulator is nelotanserin, wherein the nelotanserin is administered to pretreat between about 15 minutes and about 150 minutes prior to the compound disclosed herein (e.g., a compound of Formula (II)), including those described in Table 2. In some embodiments, the serotonin receptor modulator is nelotanserin, wherein the nelotanserin is administered to pretreat at least 180 minutes prior to the compound disclosed herein (e.g., a compound of Formula (II)), including those described in Table 2. In some embodiments, the serotonin receptor modulator is nelotanserin, wherein the nelotanserin is administered to pretreat at least 210 minutes prior to the compound disclosed herein (e.g., a compound of Formula (II)), including those described in Table 2. In some embodiments, the serotonin receptor modulator is nelotanserin, wherein the nelotanserin is administered to pretreat at least 240 minutes prior to the compound disclosed herein (e.g., a compound of Formula (II)), including those described in Table 2. In some embodiments, the serotonin receptor modulator is nelotanserin, wherein the nelotanserin is administered to pretreat at least 270 minutes prior to the compound disclosed herein (e.g., a compound of Formula (II)), including those described in Table 2.

In some embodiments, the serotonin receptor modulator is nelotanserin, wherein the nelotanserin is administered to pretreat at least 300 minutes prior to the compound disclosed herein (e.g., a compound of Formula (II)), including those described in Table 2. In some embodiments, the serotonin receptor modulator is nelotanserin, wherein the nelotanserin is administered to pretreat at least 330 minutes prior to the compound disclosed herein (e.g., a compound of Formula (II)), including those described in Table 2. In some embodiments, the serotonin receptor modulator is nelotanserin, wherein the nelotanserin is administered to pretreat at least 360 minutes prior to the compound disclosed herein (e.g., a compound of Formula (II)), including those described in Table 2. In some preferred embodiments, the serotonin receptor modulator is nelotanserin, wherein nelotanserin is administered to pretreat between about 60 minutes and about 180 minutes prior to the administration of the compound disclosed herein (e.g., a compound of Formula (II)), including those described in Table 2.

In some embodiments, the serotonin receptor modulator is pruvanserin, wherein the pruvanserin is administered to pretreat at least 15 minutes prior to the administration of the compound disclosed herein (e.g., a compound of Formula (II)), including those described in Table 2. In some embodiments, the serotonin receptor modulator is pruvanserin, wherein the pruvanserin is administered to pretreat at least 30 minutes prior to the compound disclosed herein (e.g., a compound of Formula (II)), including those described in Table 2. In some embodiments, the serotonin receptor modulator is pruvanserin, wherein the pruvanserin is administered to pretreat between at least 60 minutes and 240 minutes prior to the administration or release of the compound disclosed herein (e.g., a compound of Formula (II)), including those described in Table 2. In some embodiments, the serotonin receptor modulator is pruvanserin, wherein the pruvanserin is administered to pretreat at least 90 minutes prior to the compound disclosed herein (e.g., a compound of Formula (II)), including those described in Table 2. In some embodiments, the serotonin receptor modulator is pruvanserin, wherein the pruvanserin is administered to pretreat at least 120 minutes prior to the compound disclosed herein (e.g., a compound of Formula (II)), including those described in Table 2. In some embodiments, the serotonin receptor modulator is pruvanserin, wherein the pruvanserin is administered to pretreat at least 150 minutes prior to the compound disclosed herein (e.g., a compound of Formula (II)), including those described in Table 2. In some embodiments, the serotonin receptor modulator is pruvanserin, wherein the pruvanserin is administered to pretreat between about 15 minutes and about 150 minutes prior to the compound disclosed herein (e.g., a compound of Formula (II)), including those described in Table 2. In some embodiments, the serotonin receptor modulator is pruvanserin, wherein the pruvanserin is administered to pretreat at least 180 minutes prior to the compound disclosed herein (e.g., a compound of Formula (II)), including those described in Table 2. In some embodiments, the serotonin receptor modulator is pruvanserin, wherein the pruvanserin is administered to pretreat at least 210 minutes prior to the compound disclosed herein (e.g., a compound of Formula (II)), including those described in Table 2.

In some embodiments, the serotonin receptor modulator is pruvanserin, wherein the pruvanserin is administered to pretreat at least 240 minutes prior to the compound disclosed herein (e.g., a compound of Formula (II)), including those described in Table 2. In some embodiments, the serotonin receptor modulator is pruvanserin, wherein the pruvanserin is administered to pretreat at least 270 minutes prior to the compound disclosed herein (e.g., a compound of Formula (II)), including those described in Table 2. In some embodiments, the serotonin receptor modulator is pruvanserin, wherein the pruvanserin is administered to pretreat at least 300 minutes prior to the compound disclosed herein (e.g., a compound of Formula (II)), including those described in Table 2. In some embodiments, the serotonin receptor modulator is pruvanserin, wherein the pruvanserin is administered to pretreat at least 330 minutes prior to the compound disclosed herein (e.g., a compound of Formula (II)), including those described in Table 2. In some embodiments, the serotonin receptor modulator is pruvanserin, wherein the pruvanserin is administered to pretreat at least 360 minutes prior to the compound disclosed herein (e.g., a compound of Formula (II)), including those described in Table 2. In some preferred embodiments, the serotonin receptor modulator is pruvanserin, wherein pruvanserin is administered to pretreat between about 60 minutes and about 180 minutes prior to the administration of the compound disclosed herein (e.g., a compound of Formula (II)), including those described in Table 2.

In some embodiments, the serotonin receptor modulator is flibanserin, wherein the flibanserin is administered to pretreat at least 15 minutes prior to the administration of the compound disclosed herein (e.g., a compound of Formula (II)), including those described in Table 2. In some embodiments, the serotonin receptor modulator is flibanserin, wherein the flibanserin is administered to pretreat at least 30 minutes prior to the compound disclosed herein (e.g., a compound of Formula (II)), including those described in Table 2. In some embodiments, the serotonin receptor modulator is flibanserin, wherein the flibanserin is administered to pretreat between at least 60 minutes and 240 minutes prior to the administration or release of the compound disclosed herein (e.g., a compound of Formula (II)), including those described in Table 2. In some embodiments, the serotonin receptor modulator is flibanserin, wherein the flibanserin is administered to pretreat at least 90 minutes prior to the compound disclosed herein (e.g., a compound of Formula (II)), including those described in Table 2. In some embodiments, the serotonin receptor modulator is flibanserin, wherein the flibanserin is administered to pretreat at least 120 minutes prior to the compound disclosed herein (e.g., a compound of Formula (II)), including those described in Table 2. In some embodiments, the serotonin receptor modulator is flibanserin, wherein the flibanserin is administered to pretreat at least 150 minutes prior to the compound disclosed herein (e.g., a compound of Formula (II)), including those described in Table 2. In some embodiments, the serotonin receptor modulator is flibanserin, wherein the flibanserin is administered to pretreat between about 15 minutes and about 150 minutes prior to the compound disclosed herein (e.g., a compound of Formula (II)), including those described in Table 2. In some embodiments, the serotonin receptor modulator is flibanserin, wherein the flibanserin is administered to pretreat at least 180 minutes prior to the compound disclosed herein (e.g., a compound of Formula (II)), including those described in Table 2. In some embodiments, the serotonin receptor modulator is flibanserin, wherein the flibanserin is administered to pretreat at least 210 minutes prior to the compound disclosed herein (e.g., a compound of Formula (II)), including those described in Table 2.

In some embodiments, the serotonin receptor modulator is flibanserin, wherein the flibanserin is administered to pretreat at least 240 minutes prior to the compound disclosed herein (e.g., a compound of Formula (II)), including those described in Table 2. In some embodiments, the serotonin receptor modulator is flibanserin, wherein the flibanserin is administered to pretreat at least 270 minutes prior to the compound disclosed herein (e.g., a compound of Formula (II)), including those described in Table 2. In some embodiments, the serotonin receptor modulator is flibanserin, wherein the flibanserin is administered to pretreat at least 300 minutes prior to the compound disclosed herein (e.g., a compound of Formula (II)), including those described in Table 2. In some embodiments, the serotonin receptor modulator is flibanserin, wherein the flibanserin is administered to pretreat at least 330 minutes prior to the compound disclosed herein (e.g., a compound of Formula (II)), including those described in Table 2. In some embodiments, the serotonin receptor modulator is flibanserin, wherein the flibanserin is administered to pretreat at least 360 minutes prior to the compound disclosed herein (e.g., a compound of Formula (II)), including those described in Table 2. In some preferred embodiments, the serotonin receptor modulator is flibanserin, wherein flibanserin is administered to pretreat between about 60 minutes and about 180 minutes prior to the administration of the compound disclosed herein (e.g., a compound of Formula (II)), including those described in Table 2.

In some embodiments, the serotonin receptor modulator is olanzapine, wherein the olanzapine is administered to pretreat at least 15 minutes prior to the administration of the compound disclosed herein (e.g., a compound of Formula (II)), including those described in Table 2. In some embodiments, the serotonin receptor modulator is olanzapine, wherein the olanzapine is administered to pretreat at least 30 minutes prior to the compound disclosed herein (e.g., a compound of Formula (II)), including those described in Table 2. In some embodiments, the serotonin receptor modulator is olanzapine, wherein the olanzapine is administered to pretreat between at least 60 minutes and 240 minutes prior to the administration or release of the compound disclosed herein (e.g., a compound of Formula (II)), including those described in Table 2. In some embodiments, the serotonin receptor modulator is olanzapine, wherein the olanzapine is administered to pretreat at least 90 minutes prior to the compound disclosed herein (e.g., a compound of Formula (II)), including those described in Table 2. In some embodiments, the serotonin receptor modulator is olanzapine, wherein the olanzapine is administered to pretreat at least 120 minutes prior to the compound disclosed herein (e.g., a compound of Formula (II)), including those described in Table 2. In some embodiments, the serotonin receptor modulator is olanzapine, wherein the olanzapine is administered to pretreat at least 150 minutes prior to the compound disclosed herein (e.g., a compound of Formula (II)), including those described in Table 2. In some embodiments, the serotonin receptor modulator is olanzapine, wherein the olanzapine is administered to pretreat between about 15 minutes and about 150 minutes prior to the compound disclosed herein (e.g., a compound of Formula (II)), including those described in Table 2. In some embodiments, the serotonin receptor modulator is olanzapine, wherein the olanzapine is administered to pretreat at least 180 minutes prior to the compound disclosed herein (e.g., a compound of Formula (II)), including those described in Table 2. In some embodiments, the serotonin receptor modulator is olanzapine, wherein the olanzapine is administered to pretreat at least 210 minutes prior to the compound disclosed herein (e.g., a compound of Formula (II)), including those described in Table 2.

In some embodiments, the serotonin receptor modulator is olanzapine, wherein the olanzapine is administered to pretreat at least 240 minutes prior to the compound disclosed herein (e.g., a compound of Formula (II)), including those described in Table 2. In some embodiments, the serotonin receptor modulator is olanzapine, wherein the olanzapine is administered to pretreat at least 270 minutes prior to the compound disclosed herein (e.g., a compound of Formula (II)), including those described in Table 2. In some embodiments, the serotonin receptor modulator is olanzapine, wherein the olanzapine is administered to pretreat at least 300 minutes prior to the compound disclosed herein (e.g., a compound of Formula (II)), including those described in Table 2. In some embodiments, the serotonin receptor modulator is olanzapine, wherein the olanzapine is administered to pretreat at least 330 minutes prior to the compound disclosed herein (e.g., a compound of Formula (II)), including those described in Table 2. In some embodiments, the serotonin receptor modulator is olanzapine, wherein the olanzapine is administered to pretreat at least 360 minutes prior to the compound disclosed herein (e.g., a compound of Formula (II)), including those described in Table 2. In some preferred embodiments, the serotonin receptor modulator is olanzapine, wherein olanzapine is administered to pretreat between about 60 minutes and about 180 minutes prior to the administration of the compound disclosed herein (e.g., a compound of Formula (II)), including those described in Table 2.

In some embodiments, the serotonin receptor modulator is quetiapine, wherein the quetiapine is administered to pretreat at least 15 minutes prior to the administration of the compound disclosed herein (e.g., a compound of Formula (II)), including those described in Table 2. In some embodiments, the serotonin receptor modulator is quetiapine, wherein the quetiapine is administered to pretreat at least 30 minutes prior to the compound disclosed herein (e.g., a compound of Formula (II)), including those described in Table 2. In some embodiments, the serotonin receptor modulator is quetiapine, wherein the quetiapine is administered to pretreat between at least 60 minutes and 240 minutes prior to the administration or release of the compound disclosed herein (e.g., a compound of Formula (II)), including those described in Table 2. In some embodiments, the serotonin receptor modulator is quetiapine, wherein the quetiapine is administered to pretreat at least 90 minutes prior to the compound disclosed herein (e.g., a compound of Formula (II)), including those described in Table 2. In some embodiments, the serotonin receptor modulator is quetiapine, wherein the quetiapine is administered to pretreat at least 120 minutes prior to the compound disclosed herein (e.g., a compound of Formula (II)), including those described in Table 2. In some embodiments, the serotonin receptor modulator is quetiapine, wherein the quetiapine is administered to pretreat at least 150 minutes prior to the compound disclosed herein (e.g., a compound of Formula (II)), including those described in Table 2. In some embodiments, the serotonin receptor modulator is quetiapine, wherein the quetiapine is administered to pretreat between about 15 minutes and about 150 minutes prior to the compound disclosed herein (e.g., a compound of Formula (II)), including those described in Table 2. In some embodiments, the serotonin receptor modulator is quetiapine, wherein the quetiapine is administered to pretreat at least 180 minutes prior to the compound disclosed herein (e.g., a compound of Formula (II)), including those described in Table 2. In some embodiments, the serotonin receptor modulator is quetiapine, wherein the quetiapine is administered to pretreat at least 210 minutes prior to the compound disclosed herein (e.g., a compound of Formula (II)), including those described in Table 2.

In some embodiments, the serotonin receptor modulator is quetiapine, wherein the quetiapine is administered to pretreat at least 240 minutes prior to the compound disclosed herein (e.g., a compound of Formula (II)), including those described in Table 2. In some embodiments, the serotonin receptor modulator is quetiapine, wherein the quetiapine is administered to pretreat at least 270 minutes prior to the compound disclosed herein (e.g., a compound of Formula (II)), including those described in Table 2. In some embodiments, the serotonin receptor modulator is quetiapine, wherein the quetiapine is administered to pretreat at least 300 minutes prior to the compound disclosed herein (e.g., a compound of Formula (II)), including those described in Table 2. In some embodiments, the serotonin receptor modulator is quetiapine, wherein the quetiapine is administered to pretreat at least 330 minutes prior to the compound disclosed herein (e.g., a compound of Formula (II)), including those described in Table 2. In some embodiments, the serotonin receptor modulator is quetiapine, wherein the quetiapine is administered to pretreat at least 360 minutes prior to the compound disclosed herein (e.g., a compound of Formula (II)), including those described in Table 2. In some preferred embodiments, the serotonin receptor modulator is quetiapine, wherein quetiapine is administered to pretreat between about 60 minutes and about 180 minutes prior to the administration of the compound disclosed herein (e.g., a compound of Formula (II)), including those described in Table 2.

In some embodiments, the serotonin receptor modulator is risperidone, wherein the risperidone is administered to pretreat at least 15 minutes prior to the administration of the compound disclosed herein (e.g., a compound of Formula (II)), including those described in Table 2. In some embodiments, the serotonin receptor modulator is risperidone, wherein the risperidone is administered to pretreat at least 30 minutes prior to the compound disclosed herein (e.g., a compound of Formula (II)), including those described in Table 2. In some embodiments, the serotonin receptor modulator is risperidone, wherein the risperidone is administered to pretreat between at least 60 minutes and 240 minutes prior to the administration or release of the compound disclosed herein (e.g., a compound of Formula (II)), including those described in Table 2. In some embodiments, the serotonin receptor modulator is risperidone, wherein the risperidone is administered to pretreat at least 90 minutes prior to the compound disclosed herein (e.g., a compound of Formula (II)), including those described in Table 2. In some embodiments, the serotonin receptor modulator is risperidone, wherein the risperidone is administered to pretreat at least 120 minutes prior to the compound disclosed herein (e.g., a compound of Formula (II)), including those described in Table 2. In some embodiments, the serotonin receptor modulator is risperidone, wherein the risperidone is administered to pretreat at least 150 minutes prior to the compound disclosed herein (e.g., a compound of Formula (II)), including those described in Table 2. In some embodiments, the serotonin receptor modulator is risperidone, wherein the risperidone is administered to pretreat between about 15 minutes and about 150 minutes prior to the compound disclosed herein (e.g., a compound of Formula (II)), including those described in Table 2. In some embodiments, the serotonin receptor modulator is risperidone, wherein the risperidone is administered to pretreat at least 180 minutes prior to the compound disclosed herein (e.g., a compound of Formula (II)), including those described in Table 2. In some embodiments, the serotonin receptor modulator is risperidone, wherein the risperidone is administered to pretreat at least 210 minutes prior to the compound disclosed herein (e.g., a compound of Formula (II)), including those described in Table 2.

In some embodiments, the serotonin receptor modulator is risperidone, wherein the risperidone is administered to pretreat at least 240 minutes prior to the compound disclosed herein (e.g., a compound of Formula (II)), including those described in Table 2. In some embodiments, the serotonin receptor modulator is risperidone, wherein the risperidone is administered to pretreat at least 270 minutes prior to the compound disclosed herein (e.g., a compound of Formula (II)), including those described in Table 2. In some embodiments, the serotonin receptor modulator is risperidone, wherein the risperidone is administered to pretreat at least 300 minutes prior to the compound disclosed herein (e.g., a compound of Formula (II)), including those described in Table 2. In some embodiments, the serotonin receptor modulator is risperidone, wherein the risperidone is administered to pretreat at least 330 minutes prior to the compound disclosed herein (e.g., a compound of Formula (II)), including those described in Table 2. In some embodiments, the serotonin receptor modulator is risperidone, wherein the risperidone is administered to pretreat at least 360 minutes prior to the compound disclosed herein (e.g., a compound of Formula (II)), including those described in Table 2. In some preferred embodiments, the serotonin receptor modulator is risperidone, wherein risperidone is administered to pretreat between about 60 minutes and about 180 minutes prior to the administration of the compound disclosed herein (e.g., a compound of Formula (II)), including those described in Table 2.

In certain embodiments, such as those described above a compound disclosed herein (e.g., a compound of Formula (II)), including those described in Table 2, is co-administered with a serotonin receptor modulator in the same or in separate compositions. In one embodiment, the serotonin receptor modulator is administered after the compound disclosed herein (e.g., a compound of Formula (II)), including those described in Table 2. In one embodiment, the compound disclosed herein (e.g., a compound of Formula (II)), including those described in Table 2, is administered in a modified release formulation such that the subject is effectively post-treated with serotonin receptor modulator post to release of an effective amount of the compound. In some embodiments, the serotonin receptor modulator is part of a single fixed dose formulation that releases the compound first followed by serotonin receptor modulator on two different release profiles. In another embodiment, the compound disclosed herein (e.g., a compound of Formula (II)), including those described in Table 2, is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic.

In a preferred embodiment, the serotonin receptor modulator is administered at about 1 hour to about 3 hours after the administration of the psychedelic.

In some embodiments, the serotonin receptor modulator is eplivanserin, wherein the eplivanserin is administered to post-treat at least 15 minutes after the administration of the compound disclosed herein (e.g., a compound of Formula (II)), including those described in Table 2. In some embodiments, the serotonin receptor modulator is eplivanserin, wherein the eplivanserin is administered to post-treat between at least 30 minutes after and 360 minutes after the release or administration of the compound disclosed herein (e.g., a compound of Formula (II)), including those described in Table 2. In some embodiments, the serotonin receptor modulator is eplivanserin, wherein the eplivanserin is administered to post-treat between at least 60 minutes after and 360 minutes after the release or administration of the compound disclosed herein (e.g., a compound of Formula (II)), including those described in Table 2. In some embodiments, the serotonin receptor modulator is eplivanserin, wherein the eplivanserin is administered to post-treat at between least 90 minutes and 240 minutes after the compound disclosed herein (e.g., a compound of Formula (II)), including those described in Table 2. In some embodiments, the serotonin receptor modulator is eplivanserin, wherein the eplivanserin is administered to post-treat at least 120 minutes after the compound disclosed herein (e.g., a compound of Formula (II)), including those described in Table 2.

In some embodiments, the serotonin receptor modulator is eplivanserin, wherein the eplivanserin is administered to post-treat at least 150 minutes after the compound disclosed herein (e.g., a compound of Formula (II)), including those described in Table 2. In some embodiments, the serotonin receptor modulator is eplivanserin, wherein the eplivanserin is administered to post-treat at least 180 minutes after the compound disclosed herein (e.g., a compound of Formula (II)), including those described in Table 2. In some embodiments, the serotonin receptor modulator is eplivanserin, wherein the eplivanserin is administered to post-treat at least 210 minutes after the compound disclosed herein (e.g., a compound of Formula (II)), including those described in Table 2. In some embodiments, the serotonin receptor modulator is eplivanserin, wherein the eplivanserin is administered to post-treat at least 240 minutes after the compound disclosed herein (e.g., a compound of Formula (II)), including those described in Table 2.

In some embodiments, the serotonin receptor modulator is eplivanserin, wherein the eplivanserin is administered to post-treat at least 270 minutes after the compound disclosed herein (e.g., a compound of Formula (II)), including those described in Table 2. In some embodiments, the serotonin receptor modulator is eplivanserin, wherein the eplivanserin is administered to post-treat at least 300 minutes after the compound disclosed herein (e.g., a compound of Formula (II)), including those described in Table 2. In some embodiments, the serotonin receptor modulator is eplivanserin, wherein the eplivanserin is administered to post-treat at least 330 minutes after the compound disclosed herein (e.g., a compound of Formula (II)), including those described in Table 2. In some embodiments, the serotonin receptor modulator is eplivanserin, wherein the eplivanserin is administered to post-treat at least 360 minutes after the compound disclosed herein (e.g., a compound of Formula (II)), including those described in Table 2.

In some preferred embodiments, the serotonin receptor modulator is eplivanserin, wherein eplivanserin is administered to post-treat between about 60 minutes and about 180 minutes after the administration of the compound disclosed herein (e.g., a compound of Formula (II)), including those described in Table 2.

In some embodiments, the serotonin receptor modulator is volinanserin, wherein the volinanserin is administered to post-treat a subject between at least 15 minutes and 360 minutes after the administration or release of the compound disclosed herein (e.g., a compound of Formula (II)), including those described in Table 2. In some embodiments, the serotonin receptor modulator is volinanserin, wherein the volinanserin is administered to post-treat between at least 30 minutes and 360 minutes after the administration or release of the compound disclosed herein (e.g., a compound of Formula (II)), including those described in Table 2. In some embodiments, the serotonin receptor modulator is volinanserin, wherein the volinanserin is administered to post-treat between at least 60 minutes and 240 minutes after the administration or release of the compound disclosed herein (e.g., a compound of Formula (II)), including those described in Table 2. In some embodiments, the serotonin receptor modulator is volinanserin, wherein the volinanserin is administered to post-treat at least 90 minutes after the compound disclosed herein (e.g., a compound of Formula (II)), including those described in Table 2. In some embodiments, the serotonin receptor modulator is volinanserin, wherein the volinanserin is administered to post-treat at least 120 minutes after the compound disclosed herein (e.g., a compound of Formula (II)), including those described in Table 2.

In some embodiments, the serotonin receptor modulator is volinanserin, wherein the volinanserin is administered to post-treat at least 150 minutes after the compound disclosed herein (e.g., a compound of Formula (II)), including those described in Table 2. In some embodiments, the serotonin receptor modulator is volinanserin, wherein the volinanserin is administered to post-treat between about 15 minutes and about 150 minutes after the compound disclosed herein (e.g., a compound of Formula (II)), including those described in Table 2. In some embodiments, the serotonin receptor modulator is volinanserin, wherein the volinanserin is administered to post-treat at least 180 minutes after the compound disclosed herein (e.g., a compound of Formula (II)), including those described in Table 2. In some embodiments, the serotonin receptor modulator is volinanserin, wherein the volinanserin is administered to post-treat at least 210 minutes after the compound disclosed herein (e.g., a compound of Formula (II)), including those described in Table 2. In some embodiments, the serotonin receptor modulator is volinanserin, wherein the volinanserin is administered to post-treat at least 240 minutes after the compound disclosed herein (e.g., a compound of Formula (II)), including those described in Table 2. In some embodiments, the serotonin receptor modulator is volinanserin, wherein the volinanserin is administered to post-treat at least 270 minutes after the compound disclosed herein (e.g., a compound of Formula (II)), including those described in Table 2. In some embodiments, the serotonin receptor modulator is volinanserin, wherein the volinanserin is administered to post-treat at least 300 minutes after the compound disclosed herein (e.g., a compound of Formula (II)), including those described in Table 2. In some embodiments, the serotonin receptor modulator is volinanserin, wherein the volinanserin is administered to post-treat at least 330 minutes after the compound disclosed herein (e.g., a compound of Formula (II)), including those described in Table 2. In some embodiments, the serotonin receptor modulator is volinanserin, wherein the volinanserin is administered to post-treat at least 360 minutes after the compound disclosed herein (e.g., a compound of Formula (II)), including those described in Table 2. In some preferred embodiments, the serotonin receptor modulator is volinanserin, wherein volinanserin is administered to post-treat between about 60 minutes and about 180 minutes after the compound disclosed herein (e.g., a compound of Formula (II)), including those described in Table 2.

In some embodiments, the serotonin receptor modulator is ketanserin, wherein the ketanserin is administered to post-treat at least 15 minutes after the administration of the compound disclosed herein (e.g., a compound of Formula (II)), including those described in Table 2. In some embodiments, the serotonin receptor modulator is ketanserin, wherein the ketanserin is administered to post-treat between at least 30 minutes and 360 minutes after the administration or release of the compound disclosed herein (e.g., a compound of Formula (II)), including those described in Table 2. In some embodiments, the serotonin receptor modulator is ketanserin, wherein the ketanserin is administered to post-treat between at least 60 minutes and 240 minutes after the administration or release of the compound disclosed herein (e.g., a compound of Formula (II)), including those described in Table 2. In some embodiments, the serotonin receptor modulator is ketanserin, wherein the ketanserin is administered to post-treat at least 90 minutes after the compound disclosed herein (e.g., a compound of Formula (II)), including those described in Table 2. In some embodiments, the serotonin receptor modulator is ketanserin, wherein the ketanserin is administered to post-treat at least 120 minutes after the compound disclosed herein (e.g., a compound of Formula (II)), including those described in Table 2. In some embodiments, the serotonin receptor modulator is ketanserin, wherein the ketanserin is administered to post-treat at least 150 minutes after the compound disclosed herein (e.g., a compound of Formula (II)), including those described in Table 2. In some embodiments, the serotonin receptor modulator is ketanserin, wherein the ketanserin is administered to post-treat between about 15 minutes and about 150 minutes after the compound disclosed herein (e.g., a compound of Formula (II)), including those described in Table 2. In some embodiments, the serotonin receptor modulator is ketanserin, wherein the ketanserin is administered to post-treat at least 180 minutes after the compound disclosed herein (e.g., a compound of Formula (II)), including those described in Table 2. In some embodiments, the serotonin receptor modulator is ketanserin, wherein the ketanserin is administered to post-treat at least 210 minutes after the compound disclosed herein (e.g., a compound of Formula (II)), including those described in Table 2. In some embodiments, the serotonin receptor modulator is ketanserin, wherein the ketanserin is administered to post-treat at least 240 minutes after the compound disclosed herein (e.g., a compound of Formula (II)), including those described in Table 2. In some embodiments, the serotonin receptor modulator is ketanserin, wherein the ketanserin is administered to post-treat at least 270 minutes after the compound disclosed herein (e.g., a compound of Formula (II)), including those described in Table 2. In some embodiments, the serotonin receptor modulator is ketanserin, wherein the ketanserin is administered to post-treat at least 300 minutes after the compound disclosed herein (e.g., a compound of Formula (II)), including those described in Table 2. In some embodiments, the serotonin receptor modulator is ketanserin, wherein the ketanserin is administered to post-treat at least 330 minutes after the compound disclosed herein (e.g., a compound of Formula (II)), including those described in Table 2. In some embodiments, the serotonin receptor modulator is ketanserin, wherein the ketanserin is administered to post-treat at least 360 minutes after the compound disclosed herein (e.g., a compound of Formula (II)), including those described in Table 2. In some preferred embodiments, the serotonin receptor modulator is ketanserin, wherein ketanserin is administered to post-treat between about 60 minutes and about 180 minutes after the administration of the compound disclosed herein (e.g., a compound of Formula (II)), including those described in Table 2.

In some embodiments, the serotonin receptor modulator is ritanserin, wherein the ritanserin is administered to post-treat at least 15 minutes after the administration of the compound disclosed herein (e.g., a compound of Formula (II)), including those described in Table 2. In some embodiments, the serotonin receptor modulator is ritanserin, wherein the ritanserin is administered to post-treat at least 30 minutes after the compound disclosed herein (e.g., a compound of Formula (II)), including those described in Table 2. In some embodiments, the serotonin receptor modulator is ritanserin, wherein the ritanserin is administered to post-treat between at least 60 minutes and 240 minutes after the administration or release of the compound disclosed herein (e.g., a compound of Formula (II)), including those described in Table 2. In some embodiments, the serotonin receptor modulator is ritanserin, wherein the ritanserin is administered to post-treat at least 90 minutes after the compound disclosed herein (e.g., a compound of Formula (II)), including those described in Table 2. In some embodiments, the serotonin receptor modulator is ritanserin, wherein the ritanserin is administered to post-treat at least 120 minutes after the compound disclosed herein (e.g., a compound of Formula (II)), including those described in Table 2. In some embodiments, the serotonin receptor modulator is ritanserin, wherein the ritanserin is administered to post-treat at least 150 minutes after the compound disclosed herein (e.g., a compound of Formula (II)), including those described in Table 2. In some embodiments, the serotonin receptor modulator is ritanserin, wherein the ritanserin is administered to post-treat between about 15 minutes and about 150 minutes after the compound disclosed herein (e.g., a compound of Formula (II)), including those described in Table 2. In some embodiments, the serotonin receptor modulator is ritanserin, wherein the ritanserin is administered to post-treat at least 180 minutes after the compound disclosed herein (e.g., a compound of Formula (II)), including those described in Table 2. In some embodiments, the serotonin receptor modulator is ritanserin, wherein the ritanserin is administered to post-treat at least 210 minutes after the compound disclosed herein (e.g., a compound of Formula (II)), including those described in Table 2. In some embodiments, the serotonin receptor modulator is ritanserin, wherein the ritanserin is administered to post-treat at least 240 minutes after the compound disclosed herein (e.g., a compound of Formula (II)), including those described in Table 2. In some embodiments, the serotonin receptor modulator is ritanserin, wherein the ritanserin is administered to post-treat at least 270 minutes after the compound disclosed herein (e.g., a compound of Formula (II)), including those described in Table 2. In some embodiments, the serotonin receptor modulator is ritanserin, wherein the ritanserin is administered to post-treat at least 300 minutes after the compound disclosed herein (e.g., a compound of Formula (II)), including those described in Table 2. In some embodiments, the serotonin receptor modulator is ritanserin, wherein the ritanserin is administered to post-treat at least 330 minutes after the compound disclosed herein (e.g., a compound of Formula (II)), including those described in Table 2. In some embodiments, the serotonin receptor modulator is ritanserin, wherein the ritanserin is administered to post-treat at least 360 minutes after the compound disclosed herein (e.g., a compound of Formula (II)), including those described in Table 2. In some preferred embodiments, the serotonin receptor modulator is ritanserin, wherein ritanserin is administered to post-treat between about 60 minutes and about 180 minutes after the administration of the compound disclosed herein (e.g., a compound of Formula (II)), including those described in Table 2.

In some embodiments, the serotonin receptor modulator is pimavanserin, wherein the pimavanserin is administered to post-treat at least 15 minutes after the administration of the compound disclosed herein (e.g., a compound of Formula (II)), including those described in Table 2. In some embodiments, the serotonin receptor modulator is pimavanserin, wherein the pimavanserin is administered to post-treat at least 30 minutes after the compound disclosed herein (e.g., a compound of Formula (II)), including those described in Table 2. In some embodiments, the serotonin receptor modulator is pimavanserin, wherein the pimavanserin is administered to post-treat between at least 60 minutes and 240 minutes after the administration or release of the compound disclosed herein (e.g., a compound of Formula (II)), including those described in Table 2. In some embodiments, the serotonin receptor modulator is pimavanserin, wherein the pimavanserin is administered to post-treat at least 90 minutes after the compound disclosed herein (e.g., a compound of Formula (II)), including those described in Table 2. In some embodiments, the serotonin receptor modulator is pimavanserin, wherein the pimavanserin is administered to post-treat at least 120 minutes after the compound disclosed herein (e.g., a compound of Formula (II)), including those described in Table 2. In some embodiments, the serotonin receptor modulator is pimavanserin, wherein the pimavanserin is administered to post-treat at least 150 minutes after the compound disclosed herein (e.g., a compound of Formula (II)), including those described in Table 2. In some embodiments, the serotonin receptor modulator is pimavanserin, wherein the pimavanserin is administered to post-treat between about 15 minutes and about 150 minutes after the compound disclosed herein (e.g., a compound of Formula (II)), including those described in Table 2. In some embodiments, the serotonin receptor modulator is pimavanserin, wherein the pimavanserin is administered to post-treat at least 180 minutes after the compound disclosed herein (e.g., a compound of Formula (II)), including those described in Table 2. In some embodiments, the serotonin receptor modulator is pimavanserin, wherein the pimavanserin is administered to post-treat at least 210 minutes after the compound disclosed herein (e.g., a compound of Formula (II)), including those described in Table 2. In some embodiments, the serotonin receptor modulator is pimavanserin, wherein the pimavanserin is administered to post-treat at least 240 minutes after the compound disclosed herein (e.g., a compound of Formula (II)), including those described in Table 2. In some embodiments, the serotonin receptor modulator is pimavanserin, wherein the pimavanserin is administered to post-treat at least 270 minutes after the compound disclosed herein (e.g., a compound of Formula (II)), including those described in Table 2. In some embodiments, the serotonin receptor modulator is pimavanserin, wherein the pimavanserin is administered to post-treat at least 300 minutes after the compound disclosed herein (e.g., a compound of Formula (II)), including those described in Table 2. In some embodiments, the serotonin receptor modulator is pimavanserin, wherein the pimavanserin is administered to post-treat at least 330 minutes after the compound disclosed herein (e.g., a compound of Formula (II)), including those described in Table 2. In some embodiments, the serotonin receptor modulator is pimavanserin, wherein the pimavanserin is administered to post-treat at least 360 minutes after the compound disclosed herein (e.g., a compound of Formula (II)), including those described in Table 2. In some preferred embodiments, the serotonin receptor modulator is pimavanserin, wherein pimavanserin is administered to post-treat between about 60 minutes and about 180 minutes after the administration of the compound disclosed herein (e.g., a compound of Formula (II)), including those described in Table 2.

In some embodiments, the serotonin receptor modulator is nelotanserin, wherein the nelotanserin is administered to post-treat at least 15 minutes after the administration of the compound disclosed herein (e.g., a compound of Formula (II)), including those described in Table 2. In some embodiments, the serotonin receptor modulator is nelotanserin, wherein the nelotanserin is administered to post-treat at least 30 minutes after the compound disclosed herein (e.g., a compound of Formula (II)), including those described in Table 2. In some embodiments, the serotonin receptor modulator is nelotanserin, wherein the nelotanserin is administered to post-treat between at least 60 minutes and 240 minutes after the administration or release of the compound disclosed herein (e.g., a compound of Formula (II)), including those described in Table 2. In some embodiments, the serotonin receptor modulator is nelotanserin, wherein the nelotanserin is administered to post-treat at least 90 minutes after the compound disclosed herein (e.g., a compound of Formula (II)), including those described in Table 2. In some embodiments, the serotonin receptor modulator is nelotanserin, wherein the nelotanserin is administered to post-treat at least 120 minutes after the compound disclosed herein (e.g., a compound of Formula (II)), including those described in Table 2.

In some embodiments, the serotonin receptor modulator is nelotanserin, wherein the nelotanserin is administered to post-treat at least 150 minutes after the compound disclosed herein (e.g., a compound of Formula (II)), including those described in Table 2. In some embodiments, the serotonin receptor modulator is nelotanserin, wherein the nelotanserin is administered to post-treat between about 15 minutes and about 150 minutes after the compound disclosed herein (e.g., a compound of Formula (II)), including those described in Table 2. In some embodiments, the serotonin receptor modulator is nelotanserin, wherein the nelotanserin is administered to post-treat at least 180 minutes after the compound disclosed herein (e.g., a compound of Formula (II)), including those described in Table 2. In some embodiments, the serotonin receptor modulator is nelotanserin, wherein the nelotanserin is administered to post-treat at least 210 minutes after the compound disclosed herein (e.g., a compound of Formula (II)), including those described in Table 2. In some embodiments, the serotonin receptor modulator is nelotanserin, wherein the nelotanserin is administered to post-treat at least 240 minutes after the compound disclosed herein (e.g., a compound of Formula (II)), including those described in Table 2. In some embodiments, the serotonin receptor modulator is nelotanserin, wherein the nelotanserin is administered to post-treat at least 270 minutes after the compound disclosed herein (e.g., a compound of Formula (II)), including those described in Table 2.

In some embodiments, the serotonin receptor modulator is nelotanserin, wherein the nelotanserin is administered to post-treat at least 300 minutes after the compound disclosed herein (e.g., a compound of Formula (II)), including those described in Table 2. In some embodiments, the serotonin receptor modulator is nelotanserin, wherein the nelotanserin is administered to post-treat at least 330 minutes after the compound disclosed herein (e.g., a compound of Formula (II)), including those described in Table 2. In some embodiments, the serotonin receptor modulator is nelotanserin, wherein the nelotanserin is administered to post-treat at least 360 minutes after the compound disclosed herein (e.g., a compound of Formula (II)), including those described in Table 2. In some preferred embodiments, the serotonin receptor modulator is nelotanserin, wherein nelotanserin is administered to post-treat between about 60 minutes and about 180 minutes after the administration of the compound disclosed herein (e.g., a compound of Formula (II)), including those described in Table 2.

In some embodiments, the serotonin receptor modulator is pruvanserin, wherein the pruvanserin is administered to post-treat at least 15 minutes after the administration of the compound disclosed herein (e.g., a compound of Formula (II)), including those described in Table 2. In some embodiments, the serotonin receptor modulator is pruvanserin, wherein the pruvanserin is administered to post-treat at least 30 minutes after the compound disclosed herein (e.g., a compound of Formula (II)), including those described in Table 2. In some embodiments, the serotonin receptor modulator is pruvanserin, wherein the pruvanserin is administered to post-treat between at least 60 minutes and 240 minutes after the administration or release of the compound disclosed herein (e.g., a compound of Formula (II)), including those described in Table 2. In some embodiments, the serotonin receptor modulator is pruvanserin, wherein the pruvanserin is administered to post-treat at least 90 minutes after the compound disclosed herein (e.g., a compound of Formula (II)), including those described in Table 2. In some embodiments, the serotonin receptor modulator is pruvanserin, wherein the pruvanserin is administered to post-treat at least 120 minutes after the compound disclosed herein (e.g., a compound of Formula (II)), including those described in Table 2. In some embodiments, the serotonin receptor modulator is pruvanserin, wherein the pruvanserin is administered to post-treat at least 150 minutes after the compound disclosed herein (e.g., a compound of Formula (II)), including those described in Table 2. In some embodiments, the serotonin receptor modulator is pruvanserin, wherein the pruvanserin is administered to post-treat between about 15 minutes and about 150 minutes after the compound disclosed herein (e.g., a compound of Formula (II)), including those described in Table 2. In some embodiments, the serotonin receptor modulator is pruvanserin, wherein the pruvanserin is administered to post-treat at least 180 minutes after the compound disclosed herein (e.g., a compound of Formula (II)), including those described in Table 2. In some embodiments, the serotonin receptor modulator is pruvanserin, wherein the pruvanserin is administered to post-treat at least 210 minutes after the compound disclosed herein (e.g., a compound of Formula (II)), including those described in Table 2.

In some embodiments, the serotonin receptor modulator is pruvanserin, wherein the pruvanserin is administered to post-treat at least 240 minutes after the compound disclosed herein (e.g., a compound of Formula (II)), including those described in Table 2. In some embodiments, the serotonin receptor modulator is pruvanserin, wherein the pruvanserin is administered to post-treat at least 270 minutes after the compound disclosed herein (e.g., a compound of Formula (II)), including those described in Table 2. In some embodiments, the serotonin receptor modulator is pruvanserin, wherein the pruvanserin is administered to post-treat at least 300 minutes after the compound disclosed herein (e.g., a compound of Formula (II)), including those described in Table 2. In some embodiments, the serotonin receptor modulator is pruvanserin, wherein the pruvanserin is administered to post-treat at least 330 minutes after the compound disclosed herein (e.g., a compound of Formula (II)), including those described in Table 2. In some embodiments, the serotonin receptor modulator is pruvanserin, wherein the pruvanserin is administered to post-treat at least 360 minutes after the compound disclosed herein (e.g., a compound of Formula (II)), including those described in Table 2. In some preferred embodiments, the serotonin receptor modulator is pruvanserin, wherein pruvanserin is administered to post-treat between about 60 minutes and about 180 minutes after the administration of the compound disclosed herein (e.g., a compound of Formula (II)), including those described in Table 2.

In some embodiments, the serotonin receptor modulator is flibanserin, wherein the flibanserin is administered to post-treat at least 15 minutes post to the administration of the compound disclosed herein (e.g., a compound of Formula (II)), including those described in Table 2. In some embodiments, the serotonin receptor modulator is flibanserin, wherein the flibanserin is administered to post-treat at least 30 minutes post to the compound disclosed herein (e.g., a compound of Formula (II)), including those described in Table 2. In some embodiments, the serotonin receptor modulator is flibanserin, wherein the flibanserin is administered to post-treat between at least 60 minutes and 240 minutes post to the administration or release of the compound disclosed herein (e.g., a compound of Formula (II)), including those described in Table 2. In some embodiments, the serotonin receptor modulator is flibanserin, wherein the flibanserin is administered to post-treat at least 90 minutes post to the compound disclosed herein (e.g., a compound of Formula (II)), including those described in Table 2. In some embodiments, the serotonin receptor modulator is flibanserin, wherein the flibanserin is administered to post-treat at least 120 minutes post to the compound disclosed herein (e.g., a compound of Formula (II)), including those described in Table 2. In some embodiments, the serotonin receptor modulator is flibanserin, wherein the flibanserin is administered to post-treat at least 150 minutes post to the compound disclosed herein (e.g., a compound of Formula (II)), including those described in Table 2. In some embodiments, the serotonin receptor modulator is flibanserin, wherein the flibanserin is administered to post-treat between about 15 minutes and about 150 minutes post to the compound disclosed herein (e.g., a compound of Formula (II)), including those described in Table 2. In some embodiments, the serotonin receptor modulator is flibanserin, wherein the flibanserin is administered to post-treat at least 180 minutes post to the compound disclosed herein (e.g., a compound of Formula (II)), including those described in Table 2. In some embodiments, the serotonin receptor modulator is flibanserin, wherein the flibanserin is administered to post-treat at least 210 minutes post to the compound disclosed herein (e.g., a compound of Formula (II)), including those described in Table 2.

In some embodiments, the serotonin receptor modulator is flibanserin, wherein the flibanserin is administered to post-treat at least 240 minutes post to the compound disclosed herein (e.g., a compound of Formula (II)), including those described in Table 2. In some embodiments, the serotonin receptor modulator is flibanserin, wherein the flibanserin is administered to post-treat at least 270 minutes post to the compound disclosed herein (e.g., a compound of Formula (II)), including those described in Table 2. In some embodiments, the serotonin receptor modulator is flibanserin, wherein the flibanserin is administered to post-treat at least 300 minutes post to the compound disclosed herein (e.g., a compound of Formula (II)), including those described in Table 2. In some embodiments, the serotonin receptor modulator is flibanserin, wherein the flibanserin is administered to post-treat at least 330 minutes post to the compound disclosed herein (e.g., a compound of Formula (II)), including those described in Table 2. In some embodiments, the serotonin receptor modulator is flibanserin, wherein the flibanserin is administered to post-treat at least 360 minutes post to the compound disclosed herein (e.g., a compound of Formula (II)), including those described in Table 2. In some preferred embodiments, the serotonin receptor modulator is flibanserin, wherein flibanserin is administered to post-treat between about 60 minutes and about 180 minutes post to the administration of the compound disclosed herein (e.g., a compound of Formula (II)), including those described in Table 2.

In some embodiments, the serotonin receptor modulator is olanzapine, wherein the olanzapine is administered to post-treat at least 15 minutes post to the administration of the compound disclosed herein (e.g., a compound of Formula (II)), including those described in Table 2. In some embodiments, the serotonin receptor modulator is olanzapine, wherein the olanzapine is administered to post-treat at least 30 minutes post to the compound disclosed herein (e.g., a compound of Formula (II)), including those described in Table 2. In some embodiments, the serotonin receptor modulator is olanzapine, wherein the olanzapine is administered to post-treat between at least 60 minutes and 240 minutes post to the administration or release of the compound disclosed herein (e.g., a compound of Formula (II)), including those described in Table 2. In some embodiments, the serotonin receptor modulator is olanzapine, wherein the olanzapine is administered to post-treat at least 90 minutes post to the compound disclosed herein (e.g., a compound of Formula (II)), including those described in Table 2. In some embodiments, the serotonin receptor modulator is olanzapine, wherein the olanzapine is administered to post-treat at least 120 minutes post to the compound disclosed herein (e.g., a compound of Formula (II)), including those described in Table 2. In some embodiments, the serotonin receptor modulator is olanzapine, wherein the olanzapine is administered to post-treat at least 150 minutes post to the compound disclosed herein (e.g., a compound of Formula (II)), including those described in Table 2. In some embodiments, the serotonin receptor modulator is olanzapine, wherein the olanzapine is administered to post-treat between about 15 minutes and about 150 minutes post to the compound disclosed herein (e.g., a compound of Formula (II)), including those described in Table 2. In some embodiments, the serotonin receptor modulator is olanzapine, wherein the olanzapine is administered to post-treat at least 180 minutes post to the compound disclosed herein (e.g., a compound of Formula (II)), including those described in Table 2. In some embodiments, the serotonin receptor modulator is olanzapine, wherein the olanzapine is administered to post-treat at least 210 minutes post to the compound disclosed herein (e.g., a compound of Formula (II)), including those described in Table 2.

In some embodiments, the serotonin receptor modulator is olanzapine, wherein the olanzapine is administered to post-treat at least 240 minutes post to the compound disclosed herein (e.g., a compound of Formula (II)), including those described in Table 2. In some embodiments, the serotonin receptor modulator is olanzapine, wherein the olanzapine is administered to post-treat at least 270 minutes post to the compound disclosed herein (e.g., a compound of Formula (II)), including those described in Table 2. In some embodiments, the serotonin receptor modulator is olanzapine, wherein the olanzapine is administered to post-treat at least 300 minutes post to the compound disclosed herein (e.g., a compound of Formula (II)), including those described in Table 2. In some embodiments, the serotonin receptor modulator is olanzapine, wherein the olanzapine is administered to post-treat at least 330 minutes post to the compound disclosed herein (e.g., a compound of Formula (II)), including those described in Table 2. In some embodiments, the serotonin receptor modulator is olanzapine, wherein the olanzapine is administered to post-treat at least 360 minutes post to the compound disclosed herein (e.g., a compound of Formula (II)), including those described in Table 2. In some preferred embodiments, the serotonin receptor modulator is olanzapine, wherein olanzapine is administered to post-treat between about 60 minutes and about 180 minutes post to the administration of the compound disclosed herein (e.g., a compound of Formula (II)), including those described in Table 2.

In some embodiments, the serotonin receptor modulator is quetiapine, wherein the quetiapine is administered to post-treat at least 15 minutes post to the administration of the compound disclosed herein (e.g., a compound of Formula (II)), including those described in Table 2. In some embodiments, the serotonin receptor modulator is quetiapine, wherein the quetiapine is administered to post-treat at least 30 minutes post to the compound disclosed herein (e.g., a compound of Formula (II)), including those described in Table 2. In some embodiments, the serotonin receptor modulator is quetiapine, wherein the quetiapine is administered to post-treat between at least 60 minutes and 240 minutes post to the administration or release of the compound disclosed herein (e.g., a compound of Formula (II)), including those described in Table 2. In some embodiments, the serotonin receptor modulator is quetiapine, wherein the quetiapine is administered to post-treat at least 90 minutes post to the compound disclosed herein (e.g., a compound of Formula (II)), including those described in Table 2. In some embodiments, the serotonin receptor modulator is quetiapine, wherein the quetiapine is administered to post-treat at least 120 minutes post to the compound disclosed herein (e.g., a compound of Formula (II)), including those described in Table 2. In some embodiments, the serotonin receptor modulator is quetiapine, wherein the quetiapine is administered to post-treat at least 150 minutes post to the compound disclosed herein (e.g., a compound of Formula (II)), including those described in Table 2. In some embodiments, the serotonin receptor modulator is quetiapine, wherein the quetiapine is administered to post-treat between about 15 minutes and about 150 minutes post to the compound disclosed herein (e.g., a compound of Formula (II)), including those described in Table 2. In some embodiments, the serotonin receptor modulator is quetiapine, wherein the quetiapine is administered to post-treat at least 180 minutes post to the compound disclosed herein (e.g., a compound of Formula (II)), including those described in Table 2. In some embodiments, the serotonin receptor modulator is quetiapine, wherein the quetiapine is administered to post-treat at least 210 minutes post to the compound disclosed herein (e.g., a compound of Formula (II)), including those described in Table 2.

In some embodiments, the serotonin receptor modulator is quetiapine, wherein the quetiapine is administered to post-treat at least 240 minutes post to the compound disclosed herein (e.g., a compound of Formula (II)), including those described in Table 2. In some embodiments, the serotonin receptor modulator is quetiapine, wherein the quetiapine is administered to post-treat at least 270 minutes post to the compound disclosed herein (e.g., a compound of Formula (II)), including those described in Table 2. In some embodiments, the serotonin receptor modulator is quetiapine, wherein the quetiapine is administered to post-treat at least 300 minutes post to the compound disclosed herein (e.g., a compound of Formula (II)), including those described in Table 2. In some embodiments, the serotonin receptor modulator is quetiapine, wherein the quetiapine is administered to post-treat at least 330 minutes post to the compound disclosed herein (e.g., a compound of Formula (II)), including those described in Table 2. In some embodiments, the serotonin receptor modulator is quetiapine, wherein the quetiapine is administered to post-treat at least 360 minutes post to the compound disclosed herein (e.g., a compound of Formula (II)), including those described in Table 2. In some preferred embodiments, the serotonin receptor modulator is quetiapine, wherein quetiapine is administered to post-treat between about 60 minutes and about 180 minutes post to the administration of the compound disclosed herein (e.g., a compound of Formula (II)), including those described in Table 2.

In some embodiments, the serotonin receptor modulator is risperidone, wherein the risperidone is administered to post-treat at least 15 minutes post to the administration of the compound disclosed herein (e.g., a compound of Formula (II)), including those described in Table 2. In some embodiments, the serotonin receptor modulator is risperidone, wherein the risperidone is administered to post-treat at least 30 minutes post to the compound disclosed herein (e.g., a compound of Formula (II)), including those described in Table 2. In some embodiments, the serotonin receptor modulator is risperidone, wherein the risperidone is administered to post-treat between at least 60 minutes and 240 minutes post to the administration or release of the compound disclosed herein (e.g., a compound of Formula (II)), including those described in Table 2. In some embodiments, the serotonin receptor modulator is risperidone, wherein the risperidone is administered to post-treat at least 90 minutes post to the compound disclosed herein (e.g., a compound of Formula (II)), including those described in Table 2. In some embodiments, the serotonin receptor modulator is risperidone, wherein the risperidone is administered to post-treat at least 120 minutes post to the compound disclosed herein (e.g., a compound of Formula (II)), including those described in Table 2. In some embodiments, the serotonin receptor modulator is risperidone, wherein the risperidone is administered to post-treat at least 150 minutes post to the compound disclosed herein (e.g., a compound of Formula (II)), including those described in Table 2. In some embodiments, the serotonin receptor modulator is risperidone, wherein the risperidone is administered to post-treat between about 15 minutes and about 150 minutes post to the compound disclosed herein (e.g., a compound of Formula (II)), including those described in Table 2. In some embodiments, the serotonin receptor modulator is risperidone, wherein the risperidone is administered to post-treat at least 180 minutes post to the compound disclosed herein (e.g., a compound of Formula (II)), including those described in Table 2. In some embodiments, the serotonin receptor modulator is risperidone, wherein the risperidone is administered to post-treat at least 210 minutes post to the compound disclosed herein (e.g., a compound of Formula (II)), including those described in Table 2.

In some embodiments, the serotonin receptor modulator is risperidone, wherein the risperidone is administered to post-treat at least 240 minutes post to the compound disclosed herein (e.g., a compound of Formula (II)), including those described in Table 2. In some embodiments, the serotonin receptor modulator is risperidone, wherein the risperidone is administered to post-treat at least 270 minutes post to the compound disclosed herein (e.g., a compound of Formula (II)), including those described in Table 2. In some embodiments, the serotonin receptor modulator is risperidone, wherein the risperidone is administered to post-treat at least 300 minutes post to the compound disclosed herein (e.g., a compound of Formula (II)), including those described in Table 2. In some embodiments, the serotonin receptor modulator is risperidone, wherein the risperidone is administered to post-treat at least 330 minutes post to the compound disclosed herein (e.g., a compound of Formula (II)), including those described in Table 2. In some embodiments, the serotonin receptor modulator is risperidone, wherein the risperidone is administered to post-treat at least 360 minutes post to the compound disclosed herein (e.g., a compound of Formula (II)), including those described in Table 2. In some preferred embodiments, the serotonin receptor modulator is risperidone, wherein risperidone is administered to post-treat between about 60 minutes and about 180 minutes post to the administration of the compound disclosed herein (e.g., a compound of Formula (II)), including those described in Table 2.

In some embodiments, the serotonin receptor modulator for use with 2-(4-bromo-5-methoxy-2-(methoxy-d3)phenyl)ethan-1-amine hydrochloride is eplivanserin, wherein the eplivanserin is administered in about 1 mg to about 40 mg, or about 5 mg to about 10 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is volinanserin, wherein the volinanserin is administered in about 1 mg to about 60 mg, or about 5 mg to about 20 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is ketanserin, wherein the ketanserin is administered in about 10 mg to about 80 mg, about 30 mg to about 50 mg, or about 40 mg and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is ritanserin, wherein the ritanserin is administered in about 1 mg to about 40 mg, or about 2.5 mg to about 10 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is pimavanserin, wherein the pimavanserin is administered in about 1 mg to about 60 mg, or about 17 mg to about 34 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is nelotanserin, wherein the nelotanserin is administered in about 1 mg to about 80 mg, or about 40 mg to about 80 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is pruvanserin, wherein the pruvanserin is administered in about 1 mg to about 40 mg, or about 3 mg to about 10 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is flibanserin, wherein the flibanserin is administered in about 10 mg to about 200 mg, or about 80 mg to about 120 mg, or about 100 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is olanzapine, wherein the olanzapine is administered in about 2.5 mg to about 30 mg, or about 5 mg or about 10 mg, or about 20 mg or about 25 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is an extended-release of olanzapine such as ZYPREXA RELPREVV, wherein the extended release olanzapine is administered in about 50 mg to about 450 mg, or about 150 mg or about 210 mg, or about 300 mg or about 405 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is quetiapine, wherein the quetiapine is administered in about 25 mg to about 800 mg, or about 50 mg to about 100 mg, or about 150 mg or about 200 mg or about 250 mg or about 300 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is an extended-release of quetiapine, wherein the extended-release of quetiapine is administered in about 50 mg to about 300 mg, or about 50 mg or about 100 mg or about 200 mg, or about 300 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and 2-(4-bromo-5-methoxy-2-(methoxy-d3)phenyl)ethan-1-amine hydrochloride is administered between about 1 mg and 50 mg.

In some embodiments, the serotonin receptor modulator for use with 2-(4-bromo-5-methoxy-2-(methoxy-d3)phenyl)ethan-1-amine hydrochloride is an extended-release of risperidone including (RISPERDAL CONSTA), wherein the extended-release of risperidone is administered in about 12.5 mg, or about 25 mg, or about 37.5 mg, or about 50 mg, and 2-(4-bromo-5-methoxy-2-(methoxy-d3)phenyl)ethan-1-amine hydrochloride is administered between about 1 mg and 50 mg.

In certain embodiments, such as those described above, 2-(4-bromo-5-methoxy-2-(methoxy-d3)phenyl)ethan-1-amine hydrochloride is co-administered with a serotonin receptor modulator in the same or in separate compositions. In one embodiment, the serotonin receptor modulator is administered prior to 2-(4-bromo-5-methoxy-2-(methoxy-d3)phenyl)ethan-1-amine hydrochloride. In one embodiment, 2-(4-bromo-5-methoxy-2-(methoxy-d3)phenyl)ethan-1-amine hydrochloride is administered in a modified release formulation such that the subject is effectively pretreated with serotonin receptor modulator prior to release of an effective amount of the compound. In some embodiments the serotonin receptor modulator is part of a single fixed dose formulation that releases serotonin receptor modulator first followed by the compound on two different release profiles. In another embodiment the serotonin receptor modulator is administered first as a single dosage and after a length of time, 2-(4-bromo-5-methoxy-2-(methoxy-d3)phenyl)ethan-1-amine hydrochloride is administered as a second dosage separate from the first dosage.

In some embodiments, the serotonin receptor modulator is eplivanserin, wherein the eplivanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat between at least 30 minutes prior and 360 minutes prior to the release or administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat between at least 60 minutes prior and 360 minutes prior to the release or administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at between least 90 minutes and 240 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 120 minutes prior to 2-(4-bromo-5-methoxy-2-(methoxy-d3)phenyl)ethan-1-amine hydrochloride.

In some embodiments, the serotonin receptor modulator is eplivanserin, wherein the eplivanserin is administered to pretreat at least 150 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 180 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 210 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 240 minutes prior to 2-(4-bromo-5-methoxy-2-(methoxy-d3)phenyl)ethan-1-amine hydrochloride.

In some embodiments, the serotonin receptor modulator is eplivanserin, wherein the eplivanserin is administered to pretreat at least 270 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 300 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 330 minutes prior to 2-(4-bromo-5-methoxy-2-(methoxy-d3)phenyl)ethan-1-amine hydrochloride. In some embodiments, the serotonin receptor modulator is eplivanserin, wherein the eplivanserin is administered to pretreat at least 360 minutes prior to 2-(4-bromo-5-methoxy-2-(methoxy-d3)phenyl)ethan-1-amine hydrochloride.

In some preferred embodiments, the serotonin receptor modulator is eplivanserin, wherein eplivanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is volinanserin, wherein the volinanserin is administered to pretreat a subject between at least 15 minutes and 360 minutes prior to the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat between at least 30 minutes and 360 minutes prior to the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 90 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 120 minutes prior to 2-(4-bromo-5-methoxy-2-(methoxy-d3)phenyl)ethan-1-amine hydrochloride.

In some embodiments, the serotonin receptor modulator is volinanserin, wherein the volinanserin is administered to pretreat at least 150 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 180 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 210 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 240 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 270 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 300 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 330 minutes prior to 2-(4-bromo-5-methoxy-2-(methoxy-d3)phenyl)ethan-1-amine hydrochloride. In some embodiments, the serotonin receptor modulator is volinanserin, wherein the volinanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is volinanserin, wherein volinanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is ketanserin, wherein the ketanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat between at least 30 minutes and 360 minutes prior to the administration or release of 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 90 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 120 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 150 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 180 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 210 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 240 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 270 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 300 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 330 minutes prior to 2-(4-bromo-5-methoxy-2-(methoxy-d3)phenyl)ethan-1-amine hydrochloride. In some embodiments, the serotonin receptor modulator is ketanserin, wherein the ketanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is ketanserin, wherein ketanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is ritanserin, wherein the ritanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 30 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 90 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 120 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 150 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 180 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 210 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 240 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 270 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 300 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 330 minutes prior to 2-(4-bromo-5-methoxy-2-(methoxy-d3)phenyl)ethan-1-amine hydrochloride. In some embodiments, the serotonin receptor modulator is ritanserin, wherein the ritanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is ritanserin, wherein ritanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is pimavanserin, wherein the pimavanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 30 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 90 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 120 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 150 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 180 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 210 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 240 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 270 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 300 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 330 minutes prior to 2-(4-bromo-5-methoxy-2-(methoxy-d3)phenyl)ethan-1-amine hydrochloride. In some embodiments, the serotonin receptor modulator is pimavanserin, wherein the pimavanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is pimavanserin, wherein pimavanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is nelotanserin, wherein the nelotanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 30 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 90 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 120 minutes prior to 2-(4-bromo-5-methoxy-2-(methoxy-d3)phenyl)ethan-1-amine hydrochloride.

In some embodiments, the serotonin receptor modulator is nelotanserin, wherein the nelotanserin is administered to pretreat at least 150 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 180 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 210 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 240 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 270 minutes prior to 2-(4-bromo-5-methoxy-2-(methoxy-d3)phenyl)ethan-1-amine hydrochloride.

In some embodiments, the serotonin receptor modulator is nelotanserin, wherein the nelotanserin is administered to pretreat at least 300 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 330 minutes prior to 2-(4-bromo-5-methoxy-2-(methoxy-d3)phenyl)ethan-1-amine hydrochloride. In some embodiments, the serotonin receptor modulator is nelotanserin, wherein the nelotanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is nelotanserin, wherein nelotanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is pruvanserin, wherein the pruvanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 30 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 90 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 120 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 150 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 180 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 210 minutes prior to 2-(4-bromo-5-methoxy-2-(methoxy-d3)phenyl)ethan-1-amine hydrochloride.

In some embodiments, the serotonin receptor modulator is pruvanserin, wherein the pruvanserin is administered to pretreat at least 240 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 270 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 300 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 330 minutes prior to 2-(4-bromo-5-methoxy-2-(methoxy-d3)phenyl)ethan-1-amine hydrochloride. In some embodiments, the serotonin receptor modulator is pruvanserin, wherein the pruvanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is pruvanserin, wherein pruvanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is flibanserin, wherein the flibanserin is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 30 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 90 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 120 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 150 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 180 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 210 minutes prior to 2-(4-bromo-5-methoxy-2-(methoxy-d3)phenyl)ethan-1-amine hydrochloride.

In some embodiments, the serotonin receptor modulator is flibanserin, wherein the flibanserin is administered to pretreat at least 240 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 270 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 300 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 330 minutes prior to 2-(4-bromo-5-methoxy-2-(methoxy-d3)phenyl)ethan-1-amine hydrochloride. In some embodiments, the serotonin receptor modulator is flibanserin, wherein the flibanserin is administered to pretreat at least 360 minutes prior to 2-(4-bromo-5-methoxy-2-(methoxy-d3)phenyl)ethan-1-amine hydrochloride. In some preferred embodiments, the serotonin receptor modulator is flibanserin, wherein flibanserin is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is olanzapine, wherein the olanzapine is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 30 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 90 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 120 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 150 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 180 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 210 minutes prior to 2-(4-bromo-5-methoxy-2-(methoxy-d3)phenyl)ethan-1-amine hydrochloride.

In some embodiments, the serotonin receptor modulator is olanzapine, wherein the olanzapine is administered to pretreat at least 240 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 270 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 300 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 330 minutes prior to 2-(4-bromo-5-methoxy-2-(methoxy-d3)phenyl)ethan-1-amine hydrochloride. In some embodiments, the serotonin receptor modulator is olanzapine, wherein the olanzapine is administered to pretreat at least 360 minutes prior to 2-(4-bromo-5-methoxy-2-(methoxy-d3)phenyl)ethan-1-amine hydrochloride. In some preferred embodiments, the serotonin receptor modulator is olanzapine, wherein olanzapine is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is quetiapine, wherein the quetiapine is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 30 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 90 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 120 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 150 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 180 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 210 minutes prior to 2-(4-bromo-5-methoxy-2-(methoxy-d3)phenyl)ethan-1-amine hydrochloride.

In some embodiments, the serotonin receptor modulator is quetiapine, wherein the quetiapine is administered to pretreat at least 240 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 270 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 300 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 330 minutes prior to 2-(4-bromo-5-methoxy-2-(methoxy-d3)phenyl)ethan-1-amine hydrochloride. In some embodiments, the serotonin receptor modulator is quetiapine, wherein the quetiapine is administered to pretreat at least 360 minutes prior to 2-(4-bromo-5-methoxy-2-(methoxy-d3)phenyl)ethan-1-amine hydrochloride. In some preferred embodiments, the serotonin receptor modulator is quetiapine, wherein quetiapine is administered to pretreat between about 60 minutes and about 180 minutes prior to the administration of 2-(4-bromo-5-methoxy-2-(methoxy-d3)phenyl)ethan-1-amine hydrochloride.

In some embodiments, the serotonin receptor modulator is risperidone, wherein the risperidone is administered to pretreat at least 15 minutes prior to the administration of 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 30 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat between at least 60 minutes and 240 minutes prior to the administration or release of 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 90 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 120 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 150 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat between about 15 minutes and about 150 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 180 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 210 minutes prior to 2-(4-bromo-5-methoxy-2-(methoxy-d3)phenyl)ethan-1-amine hydrochloride.

In some embodiments, the serotonin receptor modulator is risperidone, wherein the risperidone is administered to pretreat at least 240 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 270 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 300 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 330 minutes prior to 2-(4-bromo-5-methoxy-2-(methoxy-d3)phenyl)ethan-1-amine hydrochloride. In some embodiments, the serotonin receptor modulator is risperidone, wherein the risperidone is administered to pretreat at least 360 minutes prior to 2-(4-bromo-5-methoxy-2-(methoxy-d3)phenyl)ethan-1-amine hydrochloride. In some preferred embodiments, the serotonin receptor modulator is risperidone, wherein risperidone is administered to pretreat between about 60 minutes and about 180 minutes prior to the administration of 2-(4-bromo-5-methoxy-2-(methoxy-d3)phenyl)ethan-1-amine hydrochloride.

In certain embodiments, such as those described above, 2-(4-bromo-5-methoxy-2-(methoxy-d3)phenyl)ethan-1-amine hydrochloride is co-administered with a serotonin receptor modulator in the same or in separate compositions. In one embodiment, the serotonin receptor modulator is administered after 2-(4-bromo-5-methoxy-2-(methoxy-d3)phenyl)ethan-1-amine hydrochloride. In one embodiment, 2-(4-bromo-5-methoxy-2-(methoxy-d3)phenyl)ethan-1-amine hydrochloride is administered in a modified release formulation such that the subject is effectively post-treated with serotonin receptor modulator post to release of an effective amount of the compound. In some embodiments, the serotonin receptor modulator is part of a single fixed dose formulation that releases the compound first followed by serotonin receptor modulator on two different release profiles. In another embodiment, 2-(4-bromo-5-methoxy-2-(methoxy-d3)phenyl)ethan-1-amine hydrochloride is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic (e.g., 2-(4-bromo-5-methoxy-2-(methoxy-d3)phenyl)ethan-1-amine hydrochloride).

In a preferred embodiment, the serotonin receptor modulator is administered at about 1 hour to about 3 hours after the administration of the psychedelic (e.g., 2-(4-bromo-5-methoxy-2-(methoxy-d3)phenyl)ethan-1-amine hydrochloride).

In some embodiments, the serotonin receptor modulator is eplivanserin, wherein the eplivanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat between at least 30 minutes after and 360 minutes after the release or administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat between at least 60 minutes after and 360 minutes after the release or administration of 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at between least 90 minutes and 240 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 120 minutes after 2-(4-bromo-5-methoxy-2-(methoxy-d3)phenyl)ethan-1-amine hydrochloride.

In some embodiments, the serotonin receptor modulator is eplivanserin, wherein the eplivanserin is administered to post-treat at least 150 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 180 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 210 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 240 minutes after 2-(4-bromo-5-methoxy-2-(methoxy-d3)phenyl)ethan-1-amine hydrochloride.

In some embodiments, the serotonin receptor modulator is eplivanserin, wherein the eplivanserin is administered to post-treat at least 270 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 300 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 330 minutes after 2-(4-bromo-5-methoxy-2-(methoxy-d3)phenyl)ethan-1-amine hydrochloride. In some embodiments, the serotonin receptor modulator is eplivanserin, wherein the eplivanserin is administered to post-treat at least 360 minutes after 2-(4-bromo-5-methoxy-2-(methoxy-d3)phenyl)ethan-1-amine hydrochloride.

In some preferred embodiments, the serotonin receptor modulator is eplivanserin, wherein eplivanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is volinanserin, wherein the volinanserin is administered to post-treat a subject between at least 15 minutes and 360 minutes after the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat between at least 30 minutes and 360 minutes after the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 90 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 120 minutes after 2-(4-bromo-5-methoxy-2-(methoxy-d3)phenyl)ethan-1-amine hydrochloride.

In some embodiments, the serotonin receptor modulator is volinanserin, wherein the volinanserin is administered to post-treat at least 150 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 180 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 210 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 240 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 270 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 300 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 330 minutes after 2-(4-bromo-5-methoxy-2-(methoxy-d3)phenyl)ethan-1-amine hydrochloride. In some embodiments, the serotonin receptor modulator is volinanserin, wherein the volinanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is volinanserin, wherein volinanserin is administered to post-treat between about 60 minutes and about 180 minutes after 2-(4-bromo-5-methoxy-2-(methoxy-d3)phenyl)ethan-1-amine hydrochloride.

In some embodiments, the serotonin receptor modulator is ketanserin, wherein the ketanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat between at least 30 minutes and 360 minutes after the administration or release of 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 90 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 120 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 150 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 180 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 210 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 240 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 270 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 300 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 330 minutes after 2-(4-bromo-5-methoxy-2-(methoxy-d3)phenyl)ethan-1-amine hydrochloride. In some embodiments, the serotonin receptor modulator is ketanserin, wherein the ketanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is ketanserin, wherein ketanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is ritanserin, wherein the ritanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 30 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 90 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 120 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 150 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 180 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 210 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 240 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 270 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 300 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 330 minutes after 2-(4-bromo-5-methoxy-2-(methoxy-d3)phenyl)ethan-1-amine hydrochloride. In some embodiments, the serotonin receptor modulator is ritanserin, wherein the ritanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is ritanserin, wherein ritanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is pimavanserin, wherein the pimavanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 30 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 90 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 120 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 150 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 180 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 210 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 240 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 270 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 300 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 330 minutes after 2-(4-bromo-5-methoxy-2-(methoxy-d3)phenyl)ethan-1-amine hydrochloride. In some embodiments, the serotonin receptor modulator is pimavanserin, wherein the pimavanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is pimavanserin, wherein pimavanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is nelotanserin, wherein the nelotanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 30 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 90 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 120 minutes after 2-(4-bromo-5-methoxy-2-(methoxy-d3)phenyl)ethan-1-amine hydrochloride.

In some embodiments, the serotonin receptor modulator is nelotanserin, wherein the nelotanserin is administered to post-treat at least 150 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 180 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 210 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 240 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 270 minutes after 2-(4-bromo-5-methoxy-2-(methoxy-d3)phenyl)ethan-1-amine hydrochloride.

In some embodiments, the serotonin receptor modulator is nelotanserin, wherein the nelotanserin is administered to post-treat at least 300 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 330 minutes after 2-(4-bromo-5-methoxy-2-(methoxy-d3)phenyl)ethan-1-amine hydrochloride. In some embodiments, the serotonin receptor modulator is nelotanserin, wherein the nelotanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is nelotanserin, wherein nelotanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is pruvanserin, wherein the pruvanserin is administered to post-treat at least 15 minutes after the administration of 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 30 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat between at least 60 minutes and 240 minutes after the administration or release of 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 90 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 120 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 150 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat between about 15 minutes and about 150 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 180 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 210 minutes after 2-(4-bromo-5-methoxy-2-(methoxy-d3)phenyl)ethan-1-amine hydrochloride.

In some embodiments, the serotonin receptor modulator is pruvanserin, wherein the pruvanserin is administered to post-treat at least 240 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 270 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 300 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 330 minutes after 2-(4-bromo-5-methoxy-2-(methoxy-d3)phenyl)ethan-1-amine hydrochloride. In some embodiments, the serotonin receptor modulator is pruvanserin, wherein the pruvanserin is administered to post-treat at least 360 minutes after 2-(4-bromo-5-methoxy-2-(methoxy-d3)phenyl)ethan-1-amine hydrochloride. In some preferred embodiments, the serotonin receptor modulator is pruvanserin, wherein pruvanserin is administered to post-treat between about 60 minutes and about 180 minutes after the administration of 2-(4-bromo-5-methoxy-2-(methoxy-d3)phenyl)ethan-1-amine hydrochloride.

In some embodiments, the serotonin receptor modulator is flibanserin, wherein the flibanserin is administered to post-treat at least 15 minutes post to the administration of 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 30 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat between at least 60 minutes and 240 minutes post to the administration or release of 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 90 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 120 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 150 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat between about 15 minutes and about 150 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 180 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 210 minutes post to 2-(4-bromo-5-methoxy-2-(methoxy-d3)phenyl)ethan-1-amine hydrochloride.

In some embodiments, the serotonin receptor modulator is flibanserin, wherein the flibanserin is administered to post-treat at least 240 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 270 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 300 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 330 minutes post to 2-(4-bromo-5-methoxy-2-(methoxy-d3)phenyl)ethan-1-amine hydrochloride. In some embodiments, the serotonin receptor modulator is flibanserin, wherein the flibanserin is administered to post-treat at least 360 minutes post to 2-(4-bromo-5-methoxy-2-(methoxy-d3)phenyl)ethan-1-amine hydrochloride. In some preferred embodiments, the serotonin receptor modulator is flibanserin, wherein flibanserin is administered to post-treat between about 60 minutes and about 180 minutes post to the administration of 2-(4-bromo-5-methoxy-2-(methoxy-d3)phenyl)ethan-1-amine hydrochloride.

In some embodiments, the serotonin receptor modulator is olanzapine, wherein the olanzapine is administered to post-treat at least 15 minutes post to the administration of 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 30 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat between at least 60 minutes and 240 minutes post to the administration or release of 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 90 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 120 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 150 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat between about 15 minutes and about 150 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 180 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 210 minutes post to 2-(4-bromo-5-methoxy-2-(methoxy-d3)phenyl)ethan-1-amine hydrochloride.

In some embodiments, the serotonin receptor modulator is olanzapine, wherein the olanzapine is administered to post-treat at least 240 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 270 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 300 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 330 minutes post to 2-(4-bromo-5-methoxy-2-(methoxy-d3)phenyl)ethan-1-amine hydrochloride. In some embodiments, the serotonin receptor modulator is olanzapine, wherein the olanzapine is administered to post-treat at least 360 minutes post to 2-(4-bromo-5-methoxy-2-(methoxy-d3)phenyl)ethan-1-amine hydrochloride. In some preferred embodiments, the serotonin receptor modulator is olanzapine, wherein olanzapine is administered to post-treat between about 60 minutes and about 180 minutes post to the administration of 2-(4-bromo-5-methoxy-2-(methoxy-d3)phenyl)ethan-1-amine hydrochloride.

In some embodiments, the serotonin receptor modulator is quetiapine, wherein the quetiapine is administered to post-treat at least 15 minutes post to the administration of 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 30 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat between at least 60 minutes and 240 minutes post to the administration or release of 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 90 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 120 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 150 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat between about 15 minutes and about 150 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 180 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 210 minutes post to 2-(4-bromo-5-methoxy-2-(methoxy-d3)phenyl)ethan-1-amine hydrochloride.

In some embodiments, the serotonin receptor modulator is quetiapine, wherein the quetiapine is administered to post-treat at least 240 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 270 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 300 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 330 minutes post to 2-(4-bromo-5-methoxy-2-(methoxy-d3)phenyl)ethan-1-amine hydrochloride. In some embodiments, the serotonin receptor modulator is quetiapine, wherein the quetiapine is administered to post-treat at least 360 minutes post to 2-(4-bromo-5-methoxy-2-(methoxy-d3)phenyl)ethan-1-amine hydrochloride. In some preferred embodiments, the serotonin receptor modulator is quetiapine, wherein quetiapine is administered to post-treat between about 60 minutes and about 180 minutes post to the administration of 2-(4-bromo-5-methoxy-2-(methoxy-d3)phenyl)ethan-1-amine hydrochloride.

In some embodiments, the serotonin receptor modulator is risperidone, wherein the risperidone is administered to post-treat at least 15 minutes post to the administration of 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 30 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat between at least 60 minutes and 240 minutes post to the administration or release of 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 90 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 120 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 150 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat between about 15 minutes and about 150 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 180 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 210 minutes post to 2-(4-bromo-5-methoxy-2-(methoxy-d3)phenyl)ethan-1-amine hydrochloride.

In some embodiments, the serotonin receptor modulator is risperidone, wherein the risperidone is administered to post-treat at least 240 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 270 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 300 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 330 minutes post to 2-(4-bromo-5-methoxy-2-(methoxy-d3)phenyl)ethan-1-amine hydrochloride. In some embodiments, the serotonin receptor modulator is risperidone, wherein the risperidone is administered to post-treat at least 360 minutes post to 2-(4-bromo-5-methoxy-2-(methoxy-d3)phenyl)ethan-1-amine hydrochloride. In some preferred embodiments, the serotonin receptor modulator is risperidone, wherein risperidone is administered to post-treat between about 60 minutes and about 180 minutes post to the administration of 2-(4-bromo-5-methoxy-2-(methoxy-d3)phenyl)ethan-1-amine hydrochloride.

In some embodiments, the serotonin receptor modulator for use with Compound 2 described in Table 2 is eplivanserin, wherein the eplivanserin is administered in about 1 mg to about 40 mg, or about 5 mg to about 10 mg, and Compound 2 is administered between about 1 mg and 50 mg.

In some embodiments, the serotonin receptor modulator for use with Compound 2 described in Table 2 is volinanserin, wherein the volinanserin is administered in about 1 mg to about 60 mg, or about 5 mg to about 20 mg, and Compound 2 is administered between about 1 mg and 50 mg.

In some embodiments, the serotonin receptor modulator for use with Compound 2 described in Table 2 is ketanserin, wherein the ketanserin is administered in about 10 mg to about 80 mg, about 30 mg to about 50 mg, or about 40 mg and Compound 2 is administered between about 1 mg and 50 mg.

In some embodiments, the serotonin receptor modulator for use with Compound 2 described in Table 2 is ritanserin, wherein the ritanserin is administered in about 1 mg to about 40 mg, or about 2.5 mg to about 10 mg, and Compound 2 is administered between about 1 mg and 50 mg.

In some embodiments, the serotonin receptor modulator for use with Compound 2 described in Table 2 is pimavanserin, wherein the pimavanserin is administered in about 1 mg to about 60 mg, or about 17 mg to about 34 mg, and Compound 2 described in Table 2 is administered between about 1 mg and 50 mg.

In some embodiments, the serotonin receptor modulator for use with Compound 2 described in Table 2 is nelotanserin, wherein the nelotanserin is administered in about 1 mg to about 80 mg, or about 40 mg to about 80 mg, and Compound 2 is administered between about 1 mg and 50 mg.

In some embodiments, the serotonin receptor modulator for use with Compound 2, including those described in Table 2, is pruvanserin, wherein the pruvanserin is administered in about 1 mg to about 40 mg, or about 3 mg to about 10 mg, and Compound 2 is administered between about 1 mg and 50 mg.

In some embodiments, the serotonin receptor modulator for use with Compound 2 described in Table 2 is flibanserin, wherein the flibanserin is administered in about 10 mg to about 200 mg, or about 80 mg to about 120 mg, or about 100 mg, and Compound 2 is administered between about 1 mg and 50 mg.

In some embodiments, the serotonin receptor modulator for use with Compound 2 described in Table 2 is olanzapine, wherein the olanzapine is administered in about 2.5 mg to about 30 mg, or about 5 mg or about 10 mg, or about 20 mg or about 25 mg, and Compound 2 is administered between about 1 mg and 50 mg.

In some embodiments, the serotonin receptor modulator for use with Compound 2 described in Table 2 is an extended-release of olanzapine such as ZYPREXA RELPREVV, wherein the extended release olanzapine is administered in about 50 mg to about 450 mg, or about 150 mg or about 210 mg, or about 300 mg or about 405 mg, and Compound 2 is administered between about 1 mg and 50 mg.

In some embodiments, the serotonin receptor modulator for use with Compound 2 described in Table 2 is quetiapine, wherein the quetiapine is administered in about 25 mg to about 800 mg, or about 50 mg to about 100 mg, or about 150 mg or about 200 mg or about 250 mg or about 300 mg, and Compound 2 is administered between about 1 mg and 50 mg.

In some embodiments, the serotonin receptor modulator for use with Compound 2 described in Table 2 is an extended-release of quetiapine, wherein the extended-release of quetiapine is administered in about 50 mg to about 300 mg, or about 50 mg or about 100 mg or about 200 mg, or about 300 mg, and Compound 2 is administered between about 1 mg and 50 mg.

In some embodiments, the serotonin receptor modulator for use with Compound 2 described in Table 2 is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and Compound 2 is administered between about 1 mg and 50 mg.

In some embodiments, the serotonin receptor modulator for use with Compound 2 described in Table 2 is an extended-release of risperidone including (RISPERDAL CONSTA), wherein the extended-release of risperidone is administered in about 12.5 mg, or about 25 mg, or about 37.5 mg, or about 50 mg, and Compound 2 is administered between about 1 mg and 50 mg.

In certain embodiments, such as those described above, Compound 2 described in Table 2 is co-administered with a serotonin receptor modulator in the same or in separate compositions. In one embodiment, the serotonin receptor modulator is administered prior to Compound 2. In one embodiment, Compound 2 is administered in a modified release formulation such that the subject is effectively pretreated with serotonin receptor modulator prior to release of an effective amount of the compound. In some embodiments the serotonin receptor modulator is part of a single fixed dose formulation that releases serotonin receptor modulator first followed by the compound on two different release profiles. In another embodiment the serotonin receptor modulator is administered first as a single dosage and after a length of time, Compound 2 is administered as a second dosage separate from the first dosage.

In some embodiments, the serotonin receptor modulator is eplivanserin, wherein the eplivanserin is administered to pretreat at least 15 minutes prior to the administration of Compound 2 described in Table 2. In some embodiments, the serotonin receptor modulator is eplivanserin, wherein the eplivanserin is administered to pretreat between at least 30 minutes prior and 360 minutes prior to the release or administration of Compound 2 described in Table 2. In some embodiments, the serotonin receptor modulator is eplivanserin, wherein the eplivanserin is administered to pretreat between at least 60 minutes prior and 360 minutes prior to the release or administration of Compound 2 described in Table 2. In some embodiments, the serotonin receptor modulator is eplivanserin, wherein the eplivanserin is administered to pretreat at between least 90 minutes and 240 minutes prior to Compound 2 described in Table 2. In some embodiments, the serotonin receptor modulator is eplivanserin, wherein the eplivanserin is administered to pretreat at least 120 minutes prior to Compound 2 described in Table 2.

In some embodiments, the serotonin receptor modulator is eplivanserin, wherein the eplivanserin is administered to pretreat at least 150 minutes prior to Compound 2 described in Table 2. In some embodiments, the serotonin receptor modulator is eplivanserin, wherein the eplivanserin is administered to pretreat at least 180 minutes prior to Compound 2 described in Table 2. In some embodiments, the serotonin receptor modulator is eplivanserin, wherein the eplivanserin is administered to pretreat at least 210 minutes prior to Compound 2 described in Table 2. In some embodiments, the serotonin receptor modulator is eplivanserin, wherein the eplivanserin is administered to pretreat at least 240 minutes prior to Compound 2 described in Table 2.

In some embodiments, the serotonin receptor modulator is eplivanserin, wherein the eplivanserin is administered to pretreat at least 270 minutes prior to Compound 2 described in Table 2. In some embodiments, the serotonin receptor modulator is eplivanserin, wherein the eplivanserin is administered to pretreat at least 300 minutes prior to Compound 2 described in Table 2. In some embodiments, the serotonin receptor modulator is eplivanserin, wherein the eplivanserin is administered to pretreat at least 330 minutes prior to Compound 2 described in Table 2. In some embodiments, the serotonin receptor modulator is eplivanserin, wherein the eplivanserin is administered to pretreat at least 360 minutes prior to Compound 2 described in Table 2.

In some preferred embodiments, the serotonin receptor modulator is eplivanserin, wherein eplivanserin is administered to pretreat between about 60 minutes and about 180 minutes prior to the administration of Compound 2 described in Table 2.

In some embodiments, the serotonin receptor modulator is volinanserin, wherein the volinanserin is administered to pretreat a subject between at least 15 minutes and 360 minutes prior to the administration or release of Compound 2 described in Table 2. In some embodiments, the serotonin receptor modulator is volinanserin, wherein the volinanserin is administered to pretreat between at least 30 minutes and 360 minutes prior to the administration or release of Compound 2 described in Table 2. In some embodiments, the serotonin receptor modulator is volinanserin, wherein the volinanserin is administered to pretreat between at least 60 minutes and 240 minutes prior to the administration or release of Compound 2 described in Table 2. In some embodiments, the serotonin receptor modulator is volinanserin, wherein the volinanserin is administered to pretreat at least 90 minutes prior to Compound 2 described in Table 2. In some embodiments, the serotonin receptor modulator is volinanserin, wherein the volinanserin is administered to pretreat at least 120 minutes prior to Compound 2 described in Table 2.

In some embodiments, the serotonin receptor modulator is volinanserin, wherein the volinanserin is administered to pretreat at least 150 minutes prior to Compound 2 described in Table 2. In some embodiments, the serotonin receptor modulator is volinanserin, wherein the volinanserin is administered to pretreat between about 15 minutes and about 150 minutes prior to Compound 2 described in Table 2. In some embodiments, the serotonin receptor modulator is volinanserin, wherein the volinanserin is administered to pretreat at least 180 minutes prior to Compound 2 described in Table 2. In some embodiments, the serotonin receptor modulator is volinanserin, wherein the volinanserin is administered to pretreat at least 210 minutes prior to Compound 2 described in Table 2. In some embodiments, the serotonin receptor modulator is volinanserin, wherein the volinanserin is administered to pretreat at least 240 minutes prior to Compound 2 described in Table 2. In some embodiments, the serotonin receptor modulator is volinanserin, wherein the volinanserin is administered to pretreat at least 270 minutes prior to Compound 2 described in Table 2. In some embodiments, the serotonin receptor modulator is volinanserin, wherein the volinanserin is administered to pretreat at least 300 minutes prior to Compound 2 described in Table 2. In some embodiments, the serotonin receptor modulator is volinanserin, wherein the volinanserin is administered to pretreat at least 330 minutes prior to Compound 2 described in Table 2. In some embodiments, the serotonin receptor modulator is volinanserin, wherein the volinanserin is administered to pretreat at least 360 minutes prior to Compound 2 described in Table 2. In some preferred embodiments, the serotonin receptor modulator is volinanserin, wherein volinanserin is administered to pretreat between about 60 minutes and about 180 minutes prior to the administration of Compound 2 described in Table 2.

In some embodiments, the serotonin receptor modulator is ketanserin, wherein the ketanserin is administered to pretreat at least 15 minutes prior to the administration of Compound 2 described in Table 2. In some embodiments, the serotonin receptor modulator is ketanserin, wherein the ketanserin is administered to pretreat between at least 30 minutes and 360 minutes prior to the administration or release of Compound 2 described in Table 2. In some embodiments, the serotonin receptor modulator is ketanserin, wherein the ketanserin is administered to pretreat between at least 60 minutes and 240 minutes prior to the administration or release of Compound 2 described in Table 2. In some embodiments, the serotonin receptor modulator is ketanserin, wherein the ketanserin is administered to pretreat at least 90 minutes prior to Compound 2 described in Table 2. In some embodiments, the serotonin receptor modulator is ketanserin, wherein the ketanserin is administered to pretreat at least 120 minutes prior to Compound 2 described in Table 2. In some embodiments, the serotonin receptor modulator is ketanserin, wherein the ketanserin is administered to pretreat at least 150 minutes prior to Compound 2 described in Table 2. In some embodiments, the serotonin receptor modulator is ketanserin, wherein the ketanserin is administered to pretreat between about 15 minutes and about 150 minutes prior to Compound 2 described in Table 2. In some embodiments, the serotonin receptor modulator is ketanserin, wherein the ketanserin is administered to pretreat at least 180 minutes prior to Compound 2 described in Table 2. In some embodiments, the serotonin receptor modulator is ketanserin, wherein the ketanserin is administered to pretreat at least 210 minutes prior to Compound 2 described in Table 2. In some embodiments, the serotonin receptor modulator is ketanserin, wherein the ketanserin is administered to pretreat at least 240 minutes prior to Compound 2 described in Table 2. In some embodiments, the serotonin receptor modulator is ketanserin, wherein the ketanserin is administered to pretreat at least 270 minutes prior to Compound 2 described in Table 2. In some embodiments, the serotonin receptor modulator is ketanserin, wherein the ketanserin is administered to pretreat at least 300 minutes prior to Compound 2 described in Table 2. In some embodiments, the serotonin receptor modulator is ketanserin, wherein the ketanserin is administered to pretreat at least 330 minutes prior to Compound 2 described in Table 2. In some embodiments, the serotonin receptor modulator is ketanserin, wherein the ketanserin is administered to pretreat at least 360 minutes prior to Compound 2 described in Table 2. In some preferred embodiments, the serotonin receptor modulator is ketanserin, wherein ketanserin is administered to pretreat between about 60 minutes and about 180 minutes prior to the administration of Compound 2 described in Table 2.

In some embodiments, the serotonin receptor modulator is ritanserin, wherein the ritanserin is administered to pretreat at least 15 minutes prior to the administration of Compound 2 described in Table 2. In some embodiments, the serotonin receptor modulator is ritanserin, wherein the ritanserin is administered to pretreat at least 30 minutes prior to Compound 2 described in Table 2. In some embodiments, the serotonin receptor modulator is ritanserin, wherein the ritanserin is administered to pretreat between at least 60 minutes and 240 minutes prior to the administration or release of Compound 2 described in Table 2. In some embodiments, the serotonin receptor modulator is ritanserin, wherein the ritanserin is administered to pretreat at least 90 minutes prior to Compound 2 described in Table 2. In some embodiments, the serotonin receptor modulator is ritanserin, wherein the ritanserin is administered to pretreat at least 120 minutes prior to Compound 2 described in Table 2. In some embodiments, the serotonin receptor modulator is ritanserin, wherein the ritanserin is administered to pretreat at least 150 minutes prior to Compound 2 described in Table 2. In some embodiments, the serotonin receptor modulator is ritanserin, wherein the ritanserin is administered to pretreat between about 15 minutes and about 150 minutes prior to Compound 2 described in Table 2. In some embodiments, the serotonin receptor modulator is ritanserin, wherein the ritanserin is administered to pretreat at least 180 minutes prior to Compound 2 described in Table 2. In some embodiments, the serotonin receptor modulator is ritanserin, wherein the ritanserin is administered to pretreat at least 210 minutes prior to Compound 2 described in Table 2. In some embodiments, the serotonin receptor modulator is ritanserin, wherein the ritanserin is administered to pretreat at least 240 minutes prior to Compound 2 described in Table 2. In some embodiments, the serotonin receptor modulator is ritanserin, wherein the ritanserin is administered to pretreat at least 270 minutes prior to Compound 2 described in Table 2. In some embodiments, the serotonin receptor modulator is ritanserin, wherein the ritanserin is administered to pretreat at least 300 minutes prior to Compound 2 described in Table 2. In some embodiments, the serotonin receptor modulator is ritanserin, wherein the ritanserin is administered to pretreat at least 330 minutes prior to Compound 2 described in Table 2. In some embodiments, the serotonin receptor modulator is ritanserin, wherein the ritanserin is administered to pretreat at least 360 minutes prior to Compound 2 described in Table 2. In some preferred embodiments, the serotonin receptor modulator is ritanserin, wherein ritanserin is administered to pretreat between about 60 minutes and about 180 minutes prior to the administration of Compound 2 described in Table 2.

In some embodiments, the serotonin receptor modulator is pimavanserin, wherein the pimavanserin is administered to pretreat at least 15 minutes prior to the administration of Compound 2 described in Table 2. In some embodiments, the serotonin receptor modulator is pimavanserin, wherein the pimavanserin is administered to pretreat at least 30 minutes prior to Compound 2 described in Table 2. In some embodiments, the serotonin receptor modulator is pimavanserin, wherein the pimavanserin is administered to pretreat between at least 60 minutes and 240 minutes prior to the administration or release of Compound 2 described in Table 2. In some embodiments, the serotonin receptor modulator is pimavanserin, wherein the pimavanserin is administered to pretreat at least 90 minutes prior to Compound 2 described in Table 2. In some embodiments, the serotonin receptor modulator is pimavanserin, wherein the pimavanserin is administered to pretreat at least 120 minutes prior to Compound 2 described in Table 2. In some embodiments, the serotonin receptor modulator is pimavanserin, wherein the pimavanserin is administered to pretreat at least 150 minutes prior to Compound 2 described in Table 2. In some embodiments, the serotonin receptor modulator is pimavanserin, wherein the pimavanserin is administered to pretreat between about 15 minutes and about 150 minutes prior to Compound 2 described in Table 2. In some embodiments, the serotonin receptor modulator is pimavanserin, wherein the pimavanserin is administered to pretreat at least 180 minutes prior to Compound 2 described in Table 2. In some embodiments, the serotonin receptor modulator is pimavanserin, wherein the pimavanserin is administered to pretreat at least 210 minutes prior to Compound 2 described in Table 2. In some embodiments, the serotonin receptor modulator is pimavanserin, wherein the pimavanserin is administered to pretreat at least 240 minutes prior to Compound 2 described in Table 2. In some embodiments, the serotonin receptor modulator is pimavanserin, wherein the pimavanserin is administered to pretreat at least 270 minutes prior to Compound 2 described in Table 2. In some embodiments, the serotonin receptor modulator is pimavanserin, wherein the pimavanserin is administered to pretreat at least 300 minutes prior to Compound 2 described in Table 2. In some embodiments, the serotonin receptor modulator is pimavanserin, wherein the pimavanserin is administered to pretreat at least 330 minutes prior to Compound 2 described in Table 2. In some embodiments, the serotonin receptor modulator is pimavanserin, wherein the pimavanserin is administered to pretreat at least 360 minutes prior to Compound 2 described in Table 2. In some preferred embodiments, the serotonin receptor modulator is pimavanserin, wherein pimavanserin is administered to pretreat between about 60 minutes and about 180 minutes prior to the administration of Compound 2 described in Table 2.

In some embodiments, the serotonin receptor modulator is nelotanserin, wherein the nelotanserin is administered to pretreat at least 15 minutes prior to the administration of Compound 2 described in Table 2. In some embodiments, the serotonin receptor modulator is nelotanserin, wherein the nelotanserin is administered to pretreat at least 30 minutes prior to Compound 2 described in Table 2. In some embodiments, the serotonin receptor modulator is nelotanserin, wherein the nelotanserin is administered to pretreat between at least 60 minutes and 240 minutes prior to the administration or release of Compound 2 described in Table 2. In some embodiments, the serotonin receptor modulator is nelotanserin, wherein the nelotanserin is administered to pretreat at least 90 minutes prior to Compound 2 described in Table 2. In some embodiments, the serotonin receptor modulator is nelotanserin, wherein the nelotanserin is administered to pretreat at least 120 minutes prior to Compound 2 described in Table 2.

In some embodiments, the serotonin receptor modulator is nelotanserin, wherein the nelotanserin is administered to pretreat at least 150 minutes prior to Compound 2 described in Table 2. In some embodiments, the serotonin receptor modulator is nelotanserin, wherein the nelotanserin is administered to pretreat between about 15 minutes and about 150 minutes prior to Compound 2 described in Table 2. In some embodiments, the serotonin receptor modulator is nelotanserin, wherein the nelotanserin is administered to pretreat at least 180 minutes prior to Compound 2 described in Table 2. In some embodiments, the serotonin receptor modulator is nelotanserin, wherein the nelotanserin is administered to pretreat at least 210 minutes prior to Compound 2 described in Table 2. In some embodiments, the serotonin receptor modulator is nelotanserin, wherein the nelotanserin is administered to pretreat at least 240 minutes prior to Compound 2 described in Table 2. In some embodiments, the serotonin receptor modulator is nelotanserin, wherein the nelotanserin is administered to pretreat at least 270 minutes prior to Compound 2 described in Table 2.

In some embodiments, the serotonin receptor modulator is nelotanserin, wherein the nelotanserin is administered to pretreat at least 300 minutes prior to Compound 2 described in Table 2. In some embodiments, the serotonin receptor modulator is nelotanserin, wherein the nelotanserin is administered to pretreat at least 330 minutes prior to Compound 2 described in Table 2. In some embodiments, the serotonin receptor modulator is nelotanserin, wherein the nelotanserin is administered to pretreat at least 360 minutes prior to Compound 2 described in Table 2. In some preferred embodiments, the serotonin receptor modulator is nelotanserin, wherein nelotanserin is administered to pretreat between about 60 minutes and about 180 minutes prior to the administration of Compound 2 described in Table 2.

In some embodiments, the serotonin receptor modulator is pruvanserin, wherein the pruvanserin is administered to pretreat at least 15 minutes prior to the administration of Compound 2 described in Table 2. In some embodiments, the serotonin receptor modulator is pruvanserin, wherein the pruvanserin is administered to pretreat at least 30 minutes prior to Compound 2 described in Table 2. In some embodiments, the serotonin receptor modulator is pruvanserin, wherein the pruvanserin is administered to pretreat between at least 60 minutes and 240 minutes prior to the administration or release of Compound 2 described in Table 2. In some embodiments, the serotonin receptor modulator is pruvanserin, wherein the pruvanserin is administered to pretreat at least 90 minutes prior to Compound 2 described in Table 2. In some embodiments, the serotonin receptor modulator is pruvanserin, wherein the pruvanserin is administered to pretreat at least 120 minutes prior to Compound 2 described in Table 2. In some embodiments, the serotonin receptor modulator is pruvanserin, wherein the pruvanserin is administered to pretreat at least 150 minutes prior to Compound 2 described in Table 2. In some embodiments, the serotonin receptor modulator is pruvanserin, wherein the pruvanserin is administered to pretreat between about 15 minutes and about 150 minutes prior to Compound 2 described in Table 2. In some embodiments, the serotonin receptor modulator is pruvanserin, wherein the pruvanserin is administered to pretreat at least 180 minutes prior to Compound 2 described in Table 2. In some embodiments, the serotonin receptor modulator is pruvanserin, wherein the pruvanserin is administered to pretreat at least 210 minutes prior to Compound 2 described in Table 2.

In some embodiments, the serotonin receptor modulator is pruvanserin, wherein the pruvanserin is administered to pretreat at least 240 minutes prior to Compound 2 described in Table 2. In some embodiments, the serotonin receptor modulator is pruvanserin, wherein the pruvanserin is administered to pretreat at least 270 minutes prior to Compound 2 described in Table 2. In some embodiments, the serotonin receptor modulator is pruvanserin, wherein the pruvanserin is administered to pretreat at least 300 minutes prior to Compound 2 described in Table 2. In some embodiments, the serotonin receptor modulator is pruvanserin, wherein the pruvanserin is administered to pretreat at least 330 minutes prior to Compound 2 described in Table 2. In some embodiments, the serotonin receptor modulator is pruvanserin, wherein the pruvanserin is administered to pretreat at least 360 minutes prior to Compound 2 described in Table 2. In some preferred embodiments, the serotonin receptor modulator is pruvanserin, wherein pruvanserin is administered to pretreat between about 60 minutes and about 180 minutes prior to the administration of Compound 2 described in Table 2.

In some embodiments, the serotonin receptor modulator is flibanserin, wherein the flibanserin is administered to pretreat at least 15 minutes prior to the administration of Compound 2 described in Table 2. In some embodiments, the serotonin receptor modulator is flibanserin, wherein the flibanserin is administered to pretreat at least 30 minutes prior to Compound 2 described in Table 2. In some embodiments, the serotonin receptor modulator is flibanserin, wherein the flibanserin is administered to pretreat between at least 60 minutes and 240 minutes prior to the administration or release of Compound 2 described in Table 2. In some embodiments, the serotonin receptor modulator is flibanserin, wherein the flibanserin is administered to pretreat at least 90 minutes prior to Compound 2 described in Table 2. In some embodiments, the serotonin receptor modulator is flibanserin, wherein the flibanserin is administered to pretreat at least 120 minutes prior to Compound 2 described in Table 2. In some embodiments, the serotonin receptor modulator is flibanserin, wherein the flibanserin is administered to pretreat at least 150 minutes prior to Compound 2 described in Table 2. In some embodiments, the serotonin receptor modulator is flibanserin, wherein the flibanserin is administered to pretreat between about 15 minutes and about 150 minutes prior to Compound 2 described in Table 2. In some embodiments, the serotonin receptor modulator is flibanserin, wherein the flibanserin is administered to pretreat at least 180 minutes prior to Compound 2 described in Table 2. In some embodiments, the serotonin receptor modulator is flibanserin, wherein the flibanserin is administered to pretreat at least 210 minutes prior to Compound 2 described in Table 2.

In some embodiments, the serotonin receptor modulator is flibanserin, wherein the flibanserin is administered to pretreat at least 240 minutes prior to Compound 2 described in Table 2. In some embodiments, the serotonin receptor modulator is flibanserin, wherein the flibanserin is administered to pretreat at least 270 minutes prior to Compound 2 described in Table 2. In some embodiments, the serotonin receptor modulator is flibanserin, wherein the flibanserin is administered to pretreat at least 300 minutes prior to Compound 2 described in Table 2. In some embodiments, the serotonin receptor modulator is flibanserin, wherein the flibanserin is administered to pretreat at least 330 minutes prior to Compound 2 described in Table 2. In some embodiments, the serotonin receptor modulator is flibanserin, wherein the flibanserin is administered to pretreat at least 360 minutes prior to Compound 2 described in Table 2. In some preferred embodiments, the serotonin receptor modulator is flibanserin, wherein flibanserin is administered to pretreat between about 60 minutes and about 180 minutes prior to the administration of Compound 2 described in Table 2.

In some embodiments, the serotonin receptor modulator is olanzapine, wherein the olanzapine is administered to pretreat at least 15 minutes prior to the administration of Compound 2 described in Table 2. In some embodiments, the serotonin receptor modulator is olanzapine, wherein the olanzapine is administered to pretreat at least 30 minutes prior to Compound 2 described in Table 2. In some embodiments, the serotonin receptor modulator is olanzapine, wherein the olanzapine is administered to pretreat between at least 60 minutes and 240 minutes prior to the administration or release of Compound 2 described in Table 2. In some embodiments, the serotonin receptor modulator is olanzapine, wherein the olanzapine is administered to pretreat at least 90 minutes prior to Compound 2 described in Table 2. In some embodiments, the serotonin receptor modulator is olanzapine, wherein the olanzapine is administered to pretreat at least 120 minutes prior to Compound 2 described in Table 2. In some embodiments, the serotonin receptor modulator is olanzapine, wherein the olanzapine is administered to pretreat at least 150 minutes prior to Compound 2 described in Table 2. In some embodiments, the serotonin receptor modulator is olanzapine, wherein the olanzapine is administered to pretreat between about 15 minutes and about 150 minutes prior to Compound 2 described in Table 2. In some embodiments, the serotonin receptor modulator is olanzapine, wherein the olanzapine is administered to pretreat at least 180 minutes prior to Compound 2 described in Table 2. In some embodiments, the serotonin receptor modulator is olanzapine, wherein the olanzapine is administered to pretreat at least 210 minutes prior to Compound 2 described in Table 2.

In some embodiments, the serotonin receptor modulator is olanzapine, wherein the olanzapine is administered to pretreat at least 240 minutes prior to Compound 2 described in Table 2. In some embodiments, the serotonin receptor modulator is olanzapine, wherein the olanzapine is administered to pretreat at least 270 minutes prior to Compound 2 described in Table 2. In some embodiments, the serotonin receptor modulator is olanzapine, wherein the olanzapine is administered to pretreat at least 300 minutes prior to Compound 2 described in Table 2. In some embodiments, the serotonin receptor modulator is olanzapine, wherein the olanzapine is administered to pretreat at least 330 minutes prior to Compound 2 described in Table 2. In some embodiments, the serotonin receptor modulator is olanzapine, wherein the olanzapine is administered to pretreat at least 360 minutes prior to Compound 2 described in Table 2. In some preferred embodiments, the serotonin receptor modulator is olanzapine, wherein olanzapine is administered to pretreat between about 60 minutes and about 180 minutes prior to the administration of Compound 2 described in Table 2.

In some embodiments, the serotonin receptor modulator is quetiapine, wherein the quetiapine is administered to pretreat at least 15 minutes prior to the administration of Compound 2 described in Table 2. In some embodiments, the serotonin receptor modulator is quetiapine, wherein the quetiapine is administered to pretreat at least 30 minutes prior to Compound 2 described in Table 2. In some embodiments, the serotonin receptor modulator is quetiapine, wherein the quetiapine is administered to pretreat between at least 60 minutes and 240 minutes prior to the administration or release of Compound 2 described in Table 2. In some embodiments, the serotonin receptor modulator is quetiapine, wherein the quetiapine is administered to pretreat at least 90 minutes prior to Compound 2 described in Table 2. In some embodiments, the serotonin receptor modulator is quetiapine, wherein the quetiapine is administered to pretreat at least 120 minutes prior to Compound 2 described in Table 2. In some embodiments, the serotonin receptor modulator is quetiapine, wherein the quetiapine is administered to pretreat at least 150 minutes prior to Compound 2 described in Table 2. In some embodiments, the serotonin receptor modulator is quetiapine, wherein the quetiapine is administered to pretreat between about 15 minutes and about 150 minutes prior to Compound 2 described in Table 2. In some embodiments, the serotonin receptor modulator is quetiapine, wherein the quetiapine is administered to pretreat at least 180 minutes prior to Compound 2 described in Table 2. In some embodiments, the serotonin receptor modulator is quetiapine, wherein the quetiapine is administered to pretreat at least 210 minutes prior to Compound 2 described in Table 2.

In some embodiments, the serotonin receptor modulator is quetiapine, wherein the quetiapine is administered to pretreat at least 240 minutes prior to Compound 2 described in Table 2. In some embodiments, the serotonin receptor modulator is quetiapine, wherein the quetiapine is administered to pretreat at least 270 minutes prior to Compound 2 described in Table 2. In some embodiments, the serotonin receptor modulator is quetiapine, wherein the quetiapine is administered to pretreat at least 300 minutes prior to Compound 2 described in Table 2. In some embodiments, the serotonin receptor modulator is quetiapine, wherein the quetiapine is administered to pretreat at least 330 minutes prior to Compound 2 described in Table 2. In some embodiments, the serotonin receptor modulator is quetiapine, wherein the quetiapine is administered to pretreat at least 360 minutes prior to Compound 2 described in Table 2. In some preferred embodiments, the serotonin receptor modulator is quetiapine, wherein quetiapine is administered to pretreat between about 60 minutes and about 180 minutes prior to the administration of Compound 2 described in Table 2.

In some embodiments, the serotonin receptor modulator is risperidone, wherein the risperidone is administered to pretreat at least 15 minutes prior to the administration of Compound 2 described in Table 2. In some embodiments, the serotonin receptor modulator is risperidone, wherein the risperidone is administered to pretreat at least 30 minutes prior to Compound 2 described in Table 2. In some embodiments, the serotonin receptor modulator is risperidone, wherein the risperidone is administered to pretreat between at least 60 minutes and 240 minutes prior to the administration or release of Compound 2 described in Table 2. In some embodiments, the serotonin receptor modulator is risperidone, wherein the risperidone is administered to pretreat at least 90 minutes prior to Compound 2 described in Table 2. In some embodiments, the serotonin receptor modulator is risperidone, wherein the risperidone is administered to pretreat at least 120 minutes prior to Compound 2 described in Table 2. In some embodiments, the serotonin receptor modulator is risperidone, wherein the risperidone is administered to pretreat at least 150 minutes prior to Compound 2 described in Table 2. In some embodiments, the serotonin receptor modulator is risperidone, wherein the risperidone is administered to pretreat between about 15 minutes and about 150 minutes prior to Compound 2 described in Table 2. In some embodiments, the serotonin receptor modulator is risperidone, wherein the risperidone is administered to pretreat at least 180 minutes prior to Compound 2 described in Table 2. In some embodiments, the serotonin receptor modulator is risperidone, wherein the risperidone is administered to pretreat at least 210 minutes prior to Compound 2 described in Table 2.

In some embodiments, the serotonin receptor modulator is risperidone, wherein the risperidone is administered to pretreat at least 240 minutes prior to Compound 2 described in Table 2. In some embodiments, the serotonin receptor modulator is risperidone, wherein the risperidone is administered to pretreat at least 270 minutes prior to Compound 2 described in Table 2. In some embodiments, the serotonin receptor modulator is risperidone, wherein the risperidone is administered to pretreat at least 300 minutes prior to Compound 2 described in Table 2. In some embodiments, the serotonin receptor modulator is risperidone, wherein the risperidone is administered to pretreat at least 330 minutes prior to Compound 2 described in Table 2. In some embodiments, the serotonin receptor modulator is risperidone, wherein the risperidone is administered to pretreat at least 360 minutes prior to Compound 2 described in Table 2. In some preferred embodiments, the serotonin receptor modulator is risperidone, wherein risperidone is administered to pretreat between about 60 minutes and about 180 minutes prior to the administration of Compound 2 described in Table 2.

In certain embodiments, such as those described above, Compound 2 described in Table 2, is co-administered with a serotonin receptor modulator in the same or in separate compositions. In one embodiment, the serotonin receptor modulator is administered after Compound 2. In one embodiment, Compound 2 is administered in a modified release formulation such that the subject is effectively post-treated with serotonin receptor modulator post to release of an effective amount of the compound. In some embodiments, the serotonin receptor modulator is part of a single fixed dose formulation that releases the compound first followed by serotonin receptor modulator on two different release profiles. In another embodiment, Compound 2 is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic.

In a preferred embodiment, the serotonin receptor modulator is administered at about 1 hour to about 3 hours after the administration of the psychedelic.

In some embodiments, the serotonin receptor modulator is eplivanserin, wherein the eplivanserin is administered to post-treat at least 15 minutes after the administration of Compound 2 described in Table 2. In some embodiments, the serotonin receptor modulator is eplivanserin, wherein the eplivanserin is administered to post-treat between at least 30 minutes after and 360 minutes after the release or administration of Compound 2 described in Table 2. In some embodiments, the serotonin receptor modulator is eplivanserin, wherein the eplivanserin is administered to post-treat between at least 60 minutes after and 360 minutes after the release or administration of Compound 2 described in Table 2. In some embodiments, the serotonin receptor modulator is eplivanserin, wherein the eplivanserin is administered to post-treat at between least 90 minutes and 240 minutes after Compound 2 described in Table 2. In some embodiments, the serotonin receptor modulator is eplivanserin, wherein the eplivanserin is administered to post-treat at least 120 minutes after Compound 2 described in Table 2.

In some embodiments, the serotonin receptor modulator is eplivanserin, wherein the eplivanserin is administered to post-treat at least 150 minutes after Compound 2 described in Table 2. In some embodiments, the serotonin receptor modulator is eplivanserin, wherein the eplivanserin is administered to post-treat at least 180 minutes after Compound 2 described in Table 2. In some embodiments, the serotonin receptor modulator is eplivanserin, wherein the eplivanserin is administered to post-treat at least 210 minutes after Compound 2 described in Table 2. In some embodiments, the serotonin receptor modulator is eplivanserin, wherein the eplivanserin is administered to post-treat at least 240 minutes after Compound 2 described in Table 2.

In some embodiments, the serotonin receptor modulator is eplivanserin, wherein the eplivanserin is administered to post-treat at least 270 minutes after Compound 2 described in Table 2. In some embodiments, the serotonin receptor modulator is eplivanserin, wherein the eplivanserin is administered to post-treat at least 300 minutes after Compound 2 described in Table 2. In some embodiments, the serotonin receptor modulator is eplivanserin, wherein the eplivanserin is administered to post-treat at least 330 minutes after Compound 2 described in Table 2. In some embodiments, the serotonin receptor modulator is eplivanserin, wherein the eplivanserin is administered to post-treat at least 360 minutes after Compound 2 described in Table 2.

In some preferred embodiments, the serotonin receptor modulator is eplivanserin, wherein eplivanserin is administered to post-treat between about 60 minutes and about 180 minutes after the administration of Compound 2 described in Table 2.

In some embodiments, the serotonin receptor modulator is volinanserin, wherein the volinanserin is administered to post-treat a subject between at least 15 minutes and 360 minutes after the administration or release of Compound 2 described in Table 2. In some embodiments, the serotonin receptor modulator is volinanserin, wherein the volinanserin is administered to post-treat between at least 30 minutes and 360 minutes after the administration or release of Compound 2 described in Table 2. In some embodiments, the serotonin receptor modulator is volinanserin, wherein the volinanserin is administered to post-treat between at least 60 minutes and 240 minutes after the administration or release of Compound 2 described in Table 2. In some embodiments, the serotonin receptor modulator is volinanserin, wherein the volinanserin is administered to post-treat at least 90 minutes after Compound 2 described in Table 2. In some embodiments, the serotonin receptor modulator is volinanserin, wherein the volinanserin is administered to post-treat at least 120 minutes after Compound 2 described in Table 2.

In some embodiments, the serotonin receptor modulator is volinanserin, wherein the volinanserin is administered to post-treat at least 150 minutes after Compound 2 described in Table 2. In some embodiments, the serotonin receptor modulator is volinanserin, wherein the volinanserin is administered to post-treat between about 15 minutes and about 150 minutes after Compound 2 described in Table 2. In some embodiments, the serotonin receptor modulator is volinanserin, wherein the volinanserin is administered to post-treat at least 180 minutes after Compound 2 described in Table 2. In some embodiments, the serotonin receptor modulator is volinanserin, wherein the volinanserin is administered to post-treat at least 210 minutes after Compound 2 described in Table 2. In some embodiments, the serotonin receptor modulator is volinanserin, wherein the volinanserin is administered to post-treat at least 240 minutes after Compound 2 described in Table 2. In some embodiments, the serotonin receptor modulator is volinanserin, wherein the volinanserin is administered to post-treat at least 270 minutes after Compound 2 described in Table 2. In some embodiments, the serotonin receptor modulator is volinanserin, wherein the volinanserin is administered to post-treat at least 300 minutes after Compound 2 described in Table 2. In some embodiments, the serotonin receptor modulator is volinanserin, wherein the volinanserin is administered to post-treat at least 330 minutes after Compound 2 described in Table 2. In some embodiments, the serotonin receptor modulator is volinanserin, wherein the volinanserin is administered to post-treat at least 360 minutes after Compound 2 described in Table 2. In some preferred embodiments, the serotonin receptor modulator is volinanserin, wherein volinanserin is administered to post-treat between about 60 minutes and about 180 minutes after Compound 2 described in Table 2.

In some embodiments, the serotonin receptor modulator is ketanserin, wherein the ketanserin is administered to post-treat at least 15 minutes after the administration of Compound 2 described in Table 2. In some embodiments, the serotonin receptor modulator is ketanserin, wherein the ketanserin is administered to post-treat between at least 30 minutes and 360 minutes after the administration or release of Compound 2 described in Table 2. In some embodiments, the serotonin receptor modulator is ketanserin, wherein the ketanserin is administered to post-treat between at least 60 minutes and 240 minutes after the administration or release of Compound 2 described in Table 2. In some embodiments, the serotonin receptor modulator is ketanserin, wherein the ketanserin is administered to post-treat at least 90 minutes after Compound 2 described in Table 2. In some embodiments, the serotonin receptor modulator is ketanserin, wherein the ketanserin is administered to post-treat at least 120 minutes after Compound 2 described in Table 2. In some embodiments, the serotonin receptor modulator is ketanserin, wherein the ketanserin is administered to post-treat at least 150 minutes after Compound 2 described in Table 2. In some embodiments, the serotonin receptor modulator is ketanserin, wherein the ketanserin is administered to post-treat between about 15 minutes and about 150 minutes after Compound 2 described in Table 2. In some embodiments, the serotonin receptor modulator is ketanserin, wherein the ketanserin is administered to post-treat at least 180 minutes after Compound 2 described in Table 2. In some embodiments, the serotonin receptor modulator is ketanserin, wherein the ketanserin is administered to post-treat at least 210 minutes after Compound 2 described in Table 2. In some embodiments, the serotonin receptor modulator is ketanserin, wherein the ketanserin is administered to post-treat at least 240 minutes after Compound 2 described in Table 2. In some embodiments, the serotonin receptor modulator is ketanserin, wherein the ketanserin is administered to post-treat at least 270 minutes after Compound 2 described in Table 2. In some embodiments, the serotonin receptor modulator is ketanserin, wherein the ketanserin is administered to post-treat at least 300 minutes after Compound 2 described in Table 2. In some embodiments, the serotonin receptor modulator is ketanserin, wherein the ketanserin is administered to post-treat at least 330 minutes after Compound 2 described in Table 2. In some embodiments, the serotonin receptor modulator is ketanserin, wherein the ketanserin is administered to post-treat at least 360 minutes after Compound 2 described in Table 2. In some preferred embodiments, the serotonin receptor modulator is ketanserin, wherein ketanserin is administered to post-treat between about 60 minutes and about 180 minutes after the administration of Compound 2 described in Table 2.

In some embodiments, the serotonin receptor modulator is ritanserin, wherein the ritanserin is administered to post-treat at least 15 minutes after the administration of Compound 2 described in Table 2. In some embodiments, the serotonin receptor modulator is ritanserin, wherein the ritanserin is administered to post-treat at least 30 minutes after Compound 2 described in Table 2. In some embodiments, the serotonin receptor modulator is ritanserin, wherein the ritanserin is administered to post-treat between at least 60 minutes and 240 minutes after the administration or release of Compound 2 described in Table 2. In some embodiments, the serotonin receptor modulator is ritanserin, wherein the ritanserin is administered to post-treat at least 90 minutes after Compound 2 described in Table 2. In some embodiments, the serotonin receptor modulator is ritanserin, wherein the ritanserin is administered to post-treat at least 120 minutes after Compound 2 described in Table 2. In some embodiments, the serotonin receptor modulator is ritanserin, wherein the ritanserin is administered to post-treat at least 150 minutes after Compound 2 described in Table 2. In some embodiments, the serotonin receptor modulator is ritanserin, wherein the ritanserin is administered to post-treat between about 15 minutes and about 150 minutes after Compound 2 described in Table 2. In some embodiments, the serotonin receptor modulator is ritanserin, wherein the ritanserin is administered to post-treat at least 180 minutes after Compound 2 described in Table 2. In some embodiments, the serotonin receptor modulator is ritanserin, wherein the ritanserin is administered to post-treat at least 210 minutes after Compound 2 described in Table 2. In some embodiments, the serotonin receptor modulator is ritanserin, wherein the ritanserin is administered to post-treat at least 240 minutes after Compound 2 described in Table 2. In some embodiments, the serotonin receptor modulator is ritanserin, wherein the ritanserin is administered to post-treat at least 270 minutes after Compound 2 described in Table 2. In some embodiments, the serotonin receptor modulator is ritanserin, wherein the ritanserin is administered to post-treat at least 300 minutes after Compound 2 described in Table 2. In some embodiments, the serotonin receptor modulator is ritanserin, wherein the ritanserin is administered to post-treat at least 330 minutes after Compound 2 described in Table 2. In some embodiments, the serotonin receptor modulator is ritanserin, wherein the ritanserin is administered to post-treat at least 360 minutes after Compound 2 described in Table 2. In some preferred embodiments, the serotonin receptor modulator is ritanserin, wherein ritanserin is administered to post-treat between about 60 minutes and about 180 minutes after the administration of Compound 2 described in Table 2.

In some embodiments, the serotonin receptor modulator is pimavanserin, wherein the pimavanserin is administered to post-treat at least 15 minutes after the administration of Compound 2 described in Table 2. In some embodiments, the serotonin receptor modulator is pimavanserin, wherein the pimavanserin is administered to post-treat at least 30 minutes after Compound 2 described in Table 2. In some embodiments, the serotonin receptor modulator is pimavanserin, wherein the pimavanserin is administered to post-treat between at least 60 minutes and 240 minutes after the administration or release of Compound 2 described in Table 2. In some embodiments, the serotonin receptor modulator is pimavanserin, wherein the pimavanserin is administered to post-treat at least 90 minutes after Compound 2 described in Table 2. In some embodiments, the serotonin receptor modulator is pimavanserin, wherein the pimavanserin is administered to post-treat at least 120 minutes after Compound 2 described in Table 2. In some embodiments, the serotonin receptor modulator is pimavanserin, wherein the pimavanserin is administered to post-treat at least 150 minutes after Compound 2 described in Table 2. In some embodiments, the serotonin receptor modulator is pimavanserin, wherein the pimavanserin is administered to post-treat between about 15 minutes and about 150 minutes after Compound 2 described in Table 2. In some embodiments, the serotonin receptor modulator is pimavanserin, wherein the pimavanserin is administered to post-treat at least 180 minutes after Compound 2 described in Table 2. In some embodiments, the serotonin receptor modulator is pimavanserin, wherein the pimavanserin is administered to post-treat at least 210 minutes after Compound 2 described in Table 2. In some embodiments, the serotonin receptor modulator is pimavanserin, wherein the pimavanserin is administered to post-treat at least 240 minutes after Compound 2 described in Table 2. In some embodiments, the serotonin receptor modulator is pimavanserin, wherein the pimavanserin is administered to post-treat at least 270 minutes after Compound 2 described in Table 2. In some embodiments, the serotonin receptor modulator is pimavanserin, wherein the pimavanserin is administered to post-treat at least 300 minutes after Compound 2 described in Table 2. In some embodiments, the serotonin receptor modulator is pimavanserin, wherein the pimavanserin is administered to post-treat at least 330 minutes after Compound 2 described in Table 2. In some embodiments, the serotonin receptor modulator is pimavanserin, wherein the pimavanserin is administered to post-treat at least 360 minutes after Compound 2 described in Table 2. In some preferred embodiments, the serotonin receptor modulator is pimavanserin, wherein pimavanserin is administered to post-treat between about 60 minutes and about 180 minutes after the administration of Compound 2 described in Table 2.

In some embodiments, the serotonin receptor modulator is nelotanserin, wherein the nelotanserin is administered to post-treat at least 15 minutes after the administration of Compound 2 described in Table 2. In some embodiments, the serotonin receptor modulator is nelotanserin, wherein the nelotanserin is administered to post-treat at least 30 minutes after Compound 2 described in Table 2. In some embodiments, the serotonin receptor modulator is nelotanserin, wherein the nelotanserin is administered to post-treat between at least 60 minutes and 240 minutes after the administration or release of Compound 2 described in Table 2. In some embodiments, the serotonin receptor modulator is nelotanserin, wherein the nelotanserin is administered to post-treat at least 90 minutes after Compound 2 described in Table 2. In some embodiments, the serotonin receptor modulator is nelotanserin, wherein the nelotanserin is administered to post-treat at least 120 minutes after Compound 2 described in Table 2.

In some embodiments, the serotonin receptor modulator is nelotanserin, wherein the nelotanserin is administered to post-treat at least 150 minutes after Compound 2 described in Table 2. In some embodiments, the serotonin receptor modulator is nelotanserin, wherein the nelotanserin is administered to post-treat between about 15 minutes and about 150 minutes after Compound 2 described in Table 2. In some embodiments, the serotonin receptor modulator is nelotanserin, wherein the nelotanserin is administered to post-treat at least 180 minutes after Compound 2 described in Table 2. In some embodiments, the serotonin receptor modulator is nelotanserin, wherein the nelotanserin is administered to post-treat at least 210 minutes after Compound 2 described in Table 2. In some embodiments, the serotonin receptor modulator is nelotanserin, wherein the nelotanserin is administered to post-treat at least 240 minutes after Compound 2 described in Table 2. In some embodiments, the serotonin receptor modulator is nelotanserin, wherein the nelotanserin is administered to post-treat at least 270 minutes after Compound 2 described in Table 2.

In some embodiments, the serotonin receptor modulator is nelotanserin, wherein the nelotanserin is administered to post-treat at least 300 minutes after Compound 2 described in Table 2. In some embodiments, the serotonin receptor modulator is nelotanserin, wherein the nelotanserin is administered to post-treat at least 330 minutes after Compound 2 described in Table 2. In some embodiments, the serotonin receptor modulator is nelotanserin, wherein the nelotanserin is administered to post-treat at least 360 minutes after Compound 2 described in Table 2. In some preferred embodiments, the serotonin receptor modulator is nelotanserin, wherein nelotanserin is administered to post-treat between about 60 minutes and about 180 minutes after the administration of Compound 2 described in Table 2.

In some embodiments, the serotonin receptor modulator is pruvanserin, wherein the pruvanserin is administered to post-treat at least 15 minutes after the administration of Compound 2 described in Table 2. In some embodiments, the serotonin receptor modulator is pruvanserin, wherein the pruvanserin is administered to post-treat at least 30 minutes after Compound 2 described in Table 2. In some embodiments, the serotonin receptor modulator is pruvanserin, wherein the pruvanserin is administered to post-treat between at least 60 minutes and 240 minutes after the administration or release of Compound 2 described in Table 2. In some embodiments, the serotonin receptor modulator is pruvanserin, wherein the pruvanserin is administered to post-treat at least 90 minutes after Compound 2 described in Table 2. In some embodiments, the serotonin receptor modulator is pruvanserin, wherein the pruvanserin is administered to post-treat at least 120 minutes after Compound 2 described in Table 2. In some embodiments, the serotonin receptor modulator is pruvanserin, wherein the pruvanserin is administered to post-treat at least 150 minutes after Compound 2 described in Table 2. In some embodiments, the serotonin receptor modulator is pruvanserin, wherein the pruvanserin is administered to post-treat between about 15 minutes and about 150 minutes after Compound 2 described in Table 2. In some embodiments, the serotonin receptor modulator is pruvanserin, wherein the pruvanserin is administered to post-treat at least 180 minutes after Compound 2 described in Table 2. In some embodiments, the serotonin receptor modulator is pruvanserin, wherein the pruvanserin is administered to post-treat at least 210 minutes after Compound 2 described in Table 2.

In some embodiments, the serotonin receptor modulator is pruvanserin, wherein the pruvanserin is administered to post-treat at least 240 minutes after Compound 2 described in Table 2. In some embodiments, the serotonin receptor modulator is pruvanserin, wherein the pruvanserin is administered to post-treat at least 270 minutes after Compound 2 described in Table 2. In some embodiments, the serotonin receptor modulator is pruvanserin, wherein the pruvanserin is administered to post-treat at least 300 minutes after Compound 2 described in Table 2. In some embodiments, the serotonin receptor modulator is pruvanserin, wherein the pruvanserin is administered to post-treat at least 330 minutes after Compound 2 described in Table 2. In some embodiments, the serotonin receptor modulator is pruvanserin, wherein the pruvanserin is administered to post-treat at least 360 minutes after Compound 2 described in Table 2. In some preferred embodiments, the serotonin receptor modulator is pruvanserin, wherein pruvanserin is administered to post-treat between about 60 minutes and about 180 minutes after the administration of Compound 2 described in Table 2.

In some embodiments, the serotonin receptor modulator is flibanserin, wherein the flibanserin is administered to post-treat at least 15 minutes post to the administration of Compound 2 described in Table 2. In some embodiments, the serotonin receptor modulator is flibanserin, wherein the flibanserin is administered to post-treat at least 30 minutes post to Compound 2 described in Table 2. In some embodiments, the serotonin receptor modulator is flibanserin, wherein the flibanserin is administered to post-treat between at least 60 minutes and 240 minutes post to the administration or release of Compound 2 described in Table 2. In some embodiments, the serotonin receptor modulator is flibanserin, wherein the flibanserin is administered to post-treat at least 90 minutes post to Compound 2 described in Table 2. In some embodiments, the serotonin receptor modulator is flibanserin, wherein the flibanserin is administered to post-treat at least 120 minutes post to Compound 2 described in Table 2. In some embodiments, the serotonin receptor modulator is flibanserin, wherein the flibanserin is administered to post-treat at least 150 minutes post to Compound 2 described in Table 2. In some embodiments, the serotonin receptor modulator is flibanserin, wherein the flibanserin is administered to post-treat between about 15 minutes and about 150 minutes post to Compound 2 described in Table 2. In some embodiments, the serotonin receptor modulator is flibanserin, wherein the flibanserin is administered to post-treat at least 180 minutes post to Compound 2 described in Table 2. In some embodiments, the serotonin receptor modulator is flibanserin, wherein the flibanserin is administered to post-treat at least 210 minutes post to Compound 2 described in Table 2.

In some embodiments, the serotonin receptor modulator is flibanserin, wherein the flibanserin is administered to post-treat at least 240 minutes post to Compound 2 described in Table 2. In some embodiments, the serotonin receptor modulator is flibanserin, wherein the flibanserin is administered to post-treat at least 270 minutes post to Compound 2 described in Table 2. In some embodiments, the serotonin receptor modulator is flibanserin, wherein the flibanserin is administered to post-treat at least 300 minutes post to Compound 2 described in Table 2. In some embodiments, the serotonin receptor modulator is flibanserin, wherein the flibanserin is administered to post-treat at least 330 minutes post to Compound 2 described in Table 2. In some embodiments, the serotonin receptor modulator is flibanserin, wherein the flibanserin is administered to post-treat at least 360 minutes post to Compound 2 described in Table 2. In some preferred embodiments, the serotonin receptor modulator is flibanserin, wherein flibanserin is administered to post-treat between about 60 minutes and about 180 minutes post to the administration of Compound 2 described in Table 2.

In some embodiments, the serotonin receptor modulator is olanzapine, wherein the olanzapine is administered to post-treat at least 15 minutes post to the administration of Compound 2 described in Table 2. In some embodiments, the serotonin receptor modulator is olanzapine, wherein the olanzapine is administered to post-treat at least 30 minutes post to Compound 2 described in Table 2. In some embodiments, the serotonin receptor modulator is olanzapine, wherein the olanzapine is administered to post-treat between at least 60 minutes and 240 minutes post to the administration or release of Compound 2 described in Table 2. In some embodiments, the serotonin receptor modulator is olanzapine, wherein the olanzapine is administered to post-treat at least 90 minutes post to Compound 2 described in Table 2. In some embodiments, the serotonin receptor modulator is olanzapine, wherein the olanzapine is administered to post-treat at least 120 minutes post to Compound 2 described in Table 2. In some embodiments, the serotonin receptor modulator is olanzapine, wherein the olanzapine is administered to post-treat at least 150 minutes post to Compound 2 described in Table 2. In some embodiments, the serotonin receptor modulator is olanzapine, wherein the olanzapine is administered to post-treat between about 15 minutes and about 150 minutes post to Compound 2 described in Table 2. In some embodiments, the serotonin receptor modulator is olanzapine, wherein the olanzapine is administered to post-treat at least 180 minutes post to Compound 2 described in Table 2. In some embodiments, the serotonin receptor modulator is olanzapine, wherein the olanzapine is administered to post-treat at least 210 minutes post to Compound 2 described in Table 2.

In some embodiments, the serotonin receptor modulator is olanzapine, wherein the olanzapine is administered to post-treat at least 240 minutes post to Compound 2 described in Table 2. In some embodiments, the serotonin receptor modulator is olanzapine, wherein the olanzapine is administered to post-treat at least 270 minutes post to Compound 2 described in Table 2. In some embodiments, the serotonin receptor modulator is olanzapine, wherein the olanzapine is administered to post-treat at least 300 minutes post to Compound 2 described in Table 2. In some embodiments, the serotonin receptor modulator is olanzapine, wherein the olanzapine is administered to post-treat at least 330 minutes post to Compound 2 described in Table 2. In some embodiments, the serotonin receptor modulator is olanzapine, wherein the olanzapine is administered to post-treat at least 360 minutes post to Compound 2 described in Table 2. In some preferred embodiments, the serotonin receptor modulator is olanzapine, wherein olanzapine is administered to post-treat between about 60 minutes and about 180 minutes post to the administration of Compound 2 described in Table 2.

In some embodiments, the serotonin receptor modulator is quetiapine, wherein the quetiapine is administered to post-treat at least 15 minutes post to the administration of Compound 2 described in Table 2. In some embodiments, the serotonin receptor modulator is quetiapine, wherein the quetiapine is administered to post-treat at least 30 minutes post to Compound 2 described in Table 2. In some embodiments, the serotonin receptor modulator is quetiapine, wherein the quetiapine is administered to post-treat between at least 60 minutes and 240 minutes post to the administration or release of Compound 2 described in Table 2. In some embodiments, the serotonin receptor modulator is quetiapine, wherein the quetiapine is administered to post-treat at least 90 minutes post to Compound 2 described in Table 2. In some embodiments, the serotonin receptor modulator is quetiapine, wherein the quetiapine is administered to post-treat at least 120 minutes post to Compound 2 described in Table 2. In some embodiments, the serotonin receptor modulator is quetiapine, wherein the quetiapine is administered to post-treat at least 150 minutes post to Compound 2 described in Table 2. In some embodiments, the serotonin receptor modulator is quetiapine, wherein the quetiapine is administered to post-treat between about 15 minutes and about 150 minutes post to Compound 2 described in Table 2. In some embodiments, the serotonin receptor modulator is quetiapine, wherein the quetiapine is administered to post-treat at least 180 minutes post to Compound 2 described in Table 2. In some embodiments, the serotonin receptor modulator is quetiapine, wherein the quetiapine is administered to post-treat at least 210 minutes post to Compound 2 described in Table 2.

In some embodiments, the serotonin receptor modulator is quetiapine, wherein the quetiapine is administered to post-treat at least 240 minutes post to Compound 2 described in Table 2. In some embodiments, the serotonin receptor modulator is quetiapine, wherein the quetiapine is administered to post-treat at least 270 minutes post to Compound 2 described in Table 2. In some embodiments, the serotonin receptor modulator is quetiapine, wherein the quetiapine is administered to post-treat at least 300 minutes post to Compound 2 described in Table 2. In some embodiments, the serotonin receptor modulator is quetiapine, wherein the quetiapine is administered to post-treat at least 330 minutes post to Compound 2 described in Table 2. In some embodiments, the serotonin receptor modulator is quetiapine, wherein the quetiapine is administered to post-treat at least 360 minutes post to Compound 2 described in Table 2. In some preferred embodiments, the serotonin receptor modulator is quetiapine, wherein quetiapine is administered to post-treat between about 60 minutes and about 180 minutes post to the administration of Compound 2 described in Table 2.

In some embodiments, the serotonin receptor modulator is risperidone, wherein the risperidone is administered to post-treat at least 15 minutes post to the administration of Compound 2 described in Table 2. In some embodiments, the serotonin receptor modulator is risperidone, wherein the risperidone is administered to post-treat at least 30 minutes post to Compound 2 described in Table 2. In some embodiments, the serotonin receptor modulator is risperidone, wherein the risperidone is administered to post-treat between at least 60 minutes and 240 minutes post to the administration or release of Compound 2 described in Table 2. In some embodiments, the serotonin receptor modulator is risperidone, wherein the risperidone is administered to post-treat at least 90 minutes post to Compound 2 described in Table 2. In some embodiments, the serotonin receptor modulator is risperidone, wherein the risperidone is administered to post-treat at least 120 minutes post to Compound 2 described in Table 2. In some embodiments, the serotonin receptor modulator is risperidone, wherein the risperidone is administered to post-treat at least 150 minutes post to Compound 2 described in Table 2. In some embodiments, the serotonin receptor modulator is risperidone, wherein the risperidone is administered to post-treat between about 15 minutes and about 150 minutes post to Compound 2 described in Table 2. In some embodiments, the serotonin receptor modulator is risperidone, wherein the risperidone is administered to post-treat at least 180 minutes post to Compound 2 described in Table 2. In some embodiments, the serotonin receptor modulator is risperidone, wherein the risperidone is administered to post-treat at least 210 minutes post to Compound 2 described in Table 2.

In some embodiments, the serotonin receptor modulator is risperidone, wherein the risperidone is administered to post-treat at least 240 minutes post to Compound 2 described in Table 2. In some embodiments, the serotonin receptor modulator is risperidone, wherein the risperidone is administered to post-treat at least 270 minutes post to Compound 2 described in Table 2. In some embodiments, the serotonin receptor modulator is risperidone, wherein the risperidone is administered to post-treat at least 300 minutes post to Compound 2 described in Table 2. In some embodiments, the serotonin receptor modulator is risperidone, wherein the risperidone is administered to post-treat at least 330 minutes post to Compound 2 described in Table 2. In some embodiments, the serotonin receptor modulator is risperidone, wherein the risperidone is administered to post-treat at least 360 minutes post to Compound 2 described in Table 2. In some preferred embodiments, the serotonin receptor modulator is risperidone, wherein risperidone is administered to post-treat between about 60 minutes and about 180 minutes post to the administration of Compound 2 described in Table 2.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In another aspect, provided herein are methods for increasing neuronal plasticity, comprising contacting a neuronal cell with a compound described herein (e.g., a compound of Formula (I) such as a compound of Formula (II)). In some embodiments, increasing neuronal plasticity improves a brain disorder described herein.

Also provided are methods of treating a disease or disorder in a subject in need thereof comprising administering a compound described herein (e.g., a compound of Formula (I) such as a compound of Formula (II)) in the subject, wherein the compound described herein increases neuronal plasticity in the subject. In some embodiment, the disease or disorder is neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the increased neuronal plasticity improves anti-addictive properties, antidepressant properties, anxiolytic properties, or a combination thereof. In some embodiments, the disease or disorder is a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of the compounds described herein (e.g., a compound of Formula (I) such as a compound of Formula (II)) is a phenotypic assay, a dendritogenesis assay, a spinogenesis assay, a synaptogenesis assay, a Sholl analysis, a concentration-response experiment, a 5-HT₂-A agonist assay, a 5-HT₂-A antagonist assay, a 5-HT₂-A binding assay, or a 5-HT₂-A blocking experiment (e.g., ketanserin blocking experiments). In some embodiments, the experiment or assay to determine the hallucinogenic potential of any compound of the present invention is a mouse head-twitch response (HTR) assay.

### Additional Combination Therapy

In some embodiments, the methods described herein further comprise administering one or more second therapeutic agent therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments, the second therapeutic agent is an empathogenic agent. Examples of suitable empathogenic agents for use in combination with a compound described herein (e.g., a compound of Formula (I) are selected from the phenethylamines, such as 3,4-methylene-dioxymethamphetamine (MDMA) and analogs thereof. Other suitable empathogenic agents for use in combination with the presently disclosed compounds include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha, alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha, alpha-Dimethyl-3,4-methylenedioxyphenethylamine (MDPH; 3,4-methylenedioxyphentermine)
N-Propargyl-3,4-methylenedioxyamphetamine (MDPL)
Methylenedioxy-2-aminoindane (MDAI)
1,3-Benzodioxolyl-N-methylbutanamine MBDB
N-methyl-1,3-benzodioxolylbutanamine, MBDB,
3,4-methylenedioxy-N-methyl-α-ethylphenylethylamine
3,4-Methylenedioxyamphetamine MDA
Methyl one (also known as “3,4-methylenedioxy-N-methylcathinone)
Ethylone, also known as 3,4-methylenedioxy-N-ethylcathinone
GHB or Gamma Hydroxybutyrate or sodium oxybate
N-Propyl-3,4-methylenedioxyamphetamine (MDPR).

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing Translation, Transcription, or Secretion of Neurotrophic Factors

Neurotrophic factors refer to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In another aspect, provided herein are methods for increasing at least one of translation, transcription or secretion of neurotrophic factors, comprising contacting a neuronal cell with a compound described herein (e.g., a compound of Formula (I) such as a compound of Formula (II)).

Also provided herein are methods for increase at least one of translation, transcription, or secretion of neurotrophic factors in a subject in need thereof, comprising administering to the subject a compound described herein (e.g., a compound of Formula (I) such as a compound of Formula (II)). In some embodiments, increasing at least one of translation, transcription or secretion of neurotrophic factors treats a disease or disorder such as a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMR. Abbreviations used are those conventional in the art. If not defined, the terms have their generally accepted meanings.

### Preparation of Selected Compounds and Intermediates.

The following preparations of compounds and intermediates are given to enable those of skill in the art to more clearly understand and to practice the present disclosure. They should not be considered as limiting the scope of the disclosure, but merely as illustrative and representative thereof.

### Abbreviations

app apparent
Boc tert-butyl carbamate
br broad
CCl₄ carbon tetrachloride
CDCl₃ d-chloroform
CD₃OD methanol-d4
D₂O deuterium oxide
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EDCI·HCl N-(3-Dimethylaminopropyl)-N′-ethylcarbodiimide hydrochloride
Et₂O diethyl ether
EtOAc ethyl acetate
HCl hydrochloric acid or hydrogen chloride
h hextet; sextet
HBTU O-(B enzotri azol-1-yl)-N,N,N′,N′-tetramethyluronium hexafluorophosphate
HPLC high pressure liquid chromatography
LC-MS liquid chromatography and mass spectrometry
MeOH methanol
MeCN acetonitrile
MgSO₄ magnesium sulfate
MS mass spectrometry
m multiplet
min(s) minute(s)
mL milliliter(s)
μL microliter(s)
m/z mass to charge ratio
mol mole
NHS N-Hydroxysuccinimide
p pentet
q quartet
N₂ nitrogen
NaHCO₃ sodium hydrogen carbonate
NaOH sodium hydroxide
Na₂SO₄ sodium sulfate
NH₄Cl ammonium chloride
NMP N-methyl-2-pyrrolidone
NMR nuclear magnetic resonance
Rt retention time
s singlet
t triplet
tert tertiary
TFA Trifluoroacetic acid
THF tetrahydrofuran

### Materials

The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification.

Starting material chemicals were purchased from, e.g., Sigma-Aldrich (Merck Life Science U.K. Ltd, The Old Brickyard, New Rd, Gillingham, Dorset SP8 4XT, U.K.); Fluorochem (Unit 14, Graphite Way, Hadfield, Derbyshire, SK13 1QH), and were used without further purification. Solvents were purchased as anhydrous. Petrol was the alkane fraction boiling in the range 40-60° C.

### General Conditions for Characterization

TLCs were carried out using aluminium plates pre-coated with silica gel (Kieselgel 60 F254, 0.2 mm, Merck, Darmstadt, Germany). Visualisation was by UV light.

¹H NMR spectra were recorded on a Bruker Avance BVT3200 spectrometer using the residual proton(s) in the deuterated solvents as internal standards. HPLC analyses were performed with a Shimadzu Prominence instrument (Shimadzu UK Ltd., Unit 1A Mill Court, Featherstone Road, Milton Keynes MK12 5RD, U.K.) with diode array detection and a Kinetex EVO C18, 5 μm, 250 mm×4.6 mm column.

LC-MS analyses were performed on a Shimadzu 2020 instrument operating in positive or negative ESI mode with UV detection at 254 nm.

Automated chromatography was performed on a Biotage Selekt purification system (Biotage GB Limited, Distribution Way, Dyffryn Business Park, Ystrad Mynach, Hengoed, Mid Glamorgan CF82 7TS, Wales).

Microwave reactions were performed on a Biotage Initiator ‘Sixty’ microwave reactor (Biotage GB Limited, Distribution Way, Dyffryn Business Park, Ystrad Mynach, Hengoed, Mid Glamorgan CF82 7TS, Wales).

### Example 1: Synthesis of 2-(4-bromo-2-methoxy-5-(methoxy-d3)phenyl)ethan-1,1,2,2-d₄-1-amine hydrochloride

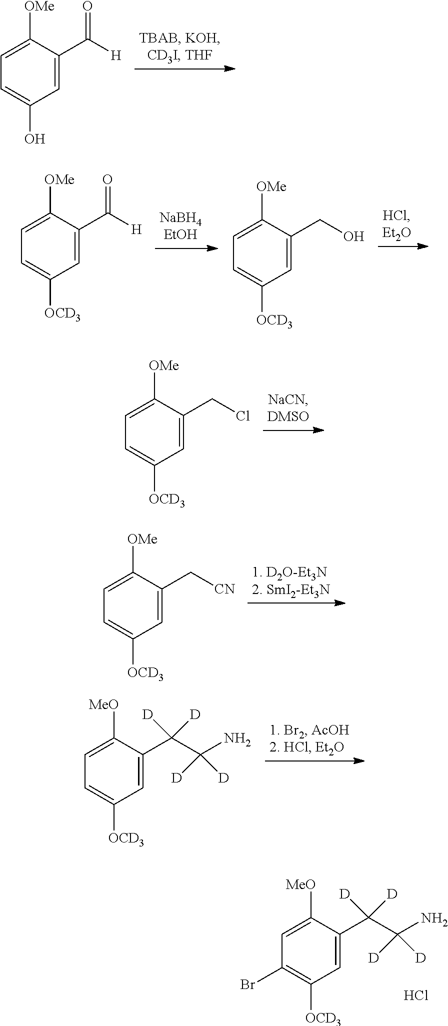

### Step 1: Preparation of 2-methoxy-5-(methoxy-d3)benzaldehyde

To 5-hydroxy-2-methoxybenzaldehyde (10.00 g, 6.6 mmol), tetra-n-butylammonium bromide (85 mg, 0.26 mmol) and ground KOH (0.56 g, 9.9 mmol) in a flask under N₂ was added iodomethane-d₃ (2.95 g, 1.3 mL, 20.4 mmol) slowly with stirring. The mixture was heated to 40° C. and stirred for 24 h, then diluted with H₂O (20 mL) and extracted with Et₂O (4×15 mL). The combined organic layers were washed with satd. brine (20 mL), dried (MgSO₄) and concentrated to give 2-methoxy-5-(methoxy-d3)benzaldehyde (1.11 g, 99%) as a solid. TLC: R_{f}=0.62 (ethyl acetate−petrol, 4:6 v/v); ¹H NMR (300 MHz, CDCl₃) δ 10.38 (s, 1H, CH), 7.26 (d, 1H, J=3.3 Hz, ArH), 7.07 (dd, 1H, J=9.0 and 3.3 Hz, ArH), 6.88 (d, 1H, J=9.0 Hz, ArH), 3.83 (s, 3H, OCH₃); ¹³C NMR: (75.5 MHz, CDCl₃) δ 189.6, 156.7, 153.6, 124.9, 123.5, 113.4, 110.4, 56.4.

### Step 2: Preparation of (2-Methoxy-5-(methoxy-d3)phenyl)methanol

To 2-methoxy-5-(methoxy-d3)benzaldehyde (400 mg, 2.36 mmol) in anhydrous EtOH (47 mL) under an atmosphere of N₂ was added NaBH₄ (135 mg, 3.55 mmol) and the mixture was heated at 80° C. for 2 h. After cooling, H₂O (30 mL) was added, the EtOH was removed in vacuo and the aqueous phase was extracted with EtOAc (4×15 mL). The combined organic layers were washed with satd. brine (15 mL), dried (MgSO₄) and concentrated to give (2-methoxy-5-(methoxy-d3)phenyl)methanol (396 mg, 98%) as an oil. TLC: R_{f}=0.31 (ethyl acetate-petrol, 3:7 v/v); ¹H NMR (300 MHz, CDCl₃) δ 6.81 (d, 1H, J=3.0 Hz, ArH), 6.72 (m, 2H, 2×ArH), 4.59 (br. s, 2H, CH₂), 3.75 (s, 3H, CH₃), 2.25 (br. s, 1H, OH); ¹³C NMR (75.5 MHz, CDCl₃) δ 153.6, 151.5, 130.1, 114.8, 113.0, 111.1, 62.2, 55.8.

### Step 3: Preparation of 2-(chloromethyl)-1-methoxy-4-(methoxy-d3)benzene

To (2-methoxy-5-(methoxy-d3)phenyl)methanol (396 g, 2.30 mmol) in Et₂O (2.3 mL) was added hydrochloric acid, 12M (2.3 mL) and the mixture was stirred at rt for 3 h. Upon completion, the mixture was diluted with H₂O (15 mL) and extracted with Et₂O (3×15 mL). The combined organic layers were washed with satd. brine (15 mL), dried (MgSO₄) and concentrated to give 2-(chloromethyl)-1-methoxy-4-(methoxy-d3)benzene (393 mg, 90%) as a solid. TLC: R_{f}=0.71 (ethyl acetate-petrol, 3:7 v/v); ¹H NMR (300 MHz, CDCl₃) δ 6.87 (m, 1H, ArH), 6.76 (m, 2H, 2×ArH), 4.56 (s, 2H, CH₂), 3.77 (s, 3H, OCH₃); ¹³C NMR (75.5 MHz, CDCl₃) δ 153.5, 151.5, 126.7, 116.2, 114.7, 112.0, 56.2, 41.5.

### Step 4: Preparation of 2-(2-methoxy-5-(methoxy-d3)phenyl)acetonitrile

To a suspension of NaCN (151 mg, 3.08 mmol) in anhydrous DMSO (1.5 mL) at rt under an atmosphere of N₂ was added a solution of 2-(chloromethyl)-1-methoxy-4-(methoxy-d₃)benzene (390 mg, 2.06 mmol) in anhydrous DMSO (2.3 mL). The mixture was heated to 80° C. and stirred for 2 h, then quenched with H₂O (20 mL) and extracted with EtOAc (3×15 mL). The combined organic layers were washed with H₂O (3×15 mL), satd. brine (15 mL), dried (MgSO₄) and concentrated to give 2-(2-methoxy-5-(methoxy-d3)phenyl)acetonitrile (324 mg, 87%) as a semi-solid. TLC: R_{f}=0.60 (ethyl acetate-petrol, 3:7 v/v); ¹H NMR (300 MHz, CDCl₃) δ 6.87 (m, 1H, ArH), 6.75 (m, 2H, 2×ArH), 3.75 (s, 3H, OCH₃), 3.60 (s, 2H, CH₂); ¹³C NMR (75.5 MHz, CDCl₃) δ 153.6, 150.9, 119.5, 117.9, 115.4, 113.8, 111.4, 55.9, 18.8.

### Step 5: Preparation of 2-(2-methoxy-5-(methoxy-d3)phenyl)ethan-1,1,2,2-d₄₋₁-amine

2-(2-Methoxy-5-(methoxy-d3)phenyl)acetonitrile (198 mg, 1.1 mmol) in anhydrous THE (1.0 mL) at rt was placed under an atmosphere of Ar, then Et₃N (1.35 g, 1.8 mL, 13.3 mmol) and D₂O (2.66 g, 2.4 mL, 133 mmol). The mixture was stirred vigorously for 16 h, then Et₃N (1.35 g, 1.8 mL, 13.3 mmol) and samarium(II) iodide solution, 0.1 M in THE (100 mL, 10 mmol) were added at rt. After 30 min, the excess samarium(II) iodide was oxidized by opening the mixture to air and stirring for 20 min. The mixture was diluted with DCM (60 mL) and 1M aqueous NaOH (60 mL). The layers were separated and the aqueous layer was extracted with DCM (3×50 mL). The combined organic layers were washed with satd. aqueous Na₂S₂O₃ (2×100 mL), dried (MgSO₄) and concentrated to give an oil. This material was dissolved in 1M HCl (2 mL) and washed with DCM (2×5 mL). The aqueous phase was basified to pH ˜11 with 15% (w/v) aqueous NaOH and extracted with DCM (4×5 mL). The combined organics were dried (MgSO₄) and concentrated to give 2-(2-methoxy-5-(methoxy-d₃)phenyl)ethan-1,1,2,2-d₄-1-amine (86 mg, 42%) as an oil. HPLC: Rₜ=4.9 min; LC-MS (+ve mode): Rₜ=3.3 min, m/z=189.15 [M+H]⁺; ¹H NMR (300 MHz, CDCl₃) δ 6.67 (m, 3H, 3×ArH), 3.70 (s, 3H, OCH₃); ¹³C NMR (75.5 MHz, CDCl₃) δ 153.4, 152.0, 129.2, 116.9, 111.3, 55.9.

### Step 6: Preparation of 2-(4-bromo-2-methoxy-5-(methoxy-d3)phenyl)ethan-1,1,2,2-d₄-1-amine hydrochloride

To a stirred mixture of 2-(2-methoxy-5-(methoxy-d3)phenyl)ethan-1,1,2,2-d₄-1-amine (86 mg, 0.46 mmol) in AcOH (100 μL) at rt was added a solution of bromine (80 mg, 26 μL, 0.50 mmol) in AcOH (100 μL) dropwise. The resulting mixture was stirred at rt for 10 min, then diluted with Et₂O (5 mL) followed by 10% (w/v) aqueous Na₂S₂O₃ (5 mL) and stirred for 30 min. A solution of 15% (w/v) aqueous NaOH was added to obtain pH ˜11, the mixture was extracted with DCM (4×5 mL) and the combined organic layers were dried (MgSO₄) and concentrated to give an oil. The residue was stirred in a solution of 2M HCl in Et₂O (1.5 mL, 3.0 mmol) for 40 min. The solid was collected by filtration and the filter cake was washed with Et₂O (5×3 mL) to give impure 2-(4-bromo-2-methoxy-5-(methoxy-d₃)phenyl)ethan-1,1,2,2-d₄-1-amine hydrochloride (90 mg) as a solid. This material was purified by reversed-phase chromatography, eluting with 0 to 100% MeOH in 0.02% hydrochloric acid to give 2-(4-bromo-2-methoxy-5-(methoxy-d3)phenyl)ethan-1,1,2,2-d₄-1-amine hydrochloride (34 mg, 28%) as a solid. HPLC: Rₜ=12.1 min; LC-MS (+ve mode): Rₜ=7.3 min, m/z=267.05 and 269.05 [M+H]⁺; ¹H NMR (300 MHz, CD₃OD) δ 7.20 (s, 1H, ArH), 6.96 (s, 1H, ArH), 3.84 (s, 3H, OCH₃); ¹³C NMR (75.5 MHz, CD₃OD) δ 152.0, 150.3, 124.7, 115.5, 114.8, 110.1, 55.2.

### Example 2: Synthesis of 2-(4-bromo-2-methoxy-5-(methoxy-d3)phenyl)ethan-1,1-d₂-1-amine hydrochloride

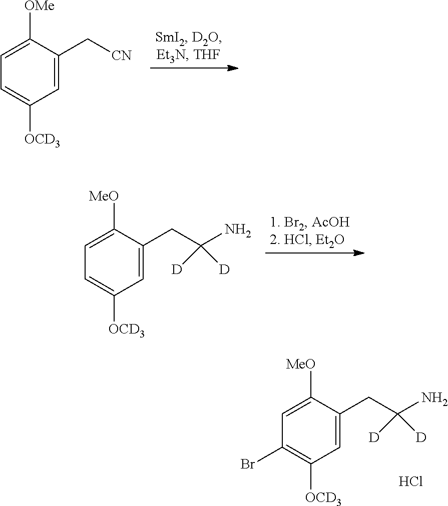

### Step 1: Preparation of 2-(2-methoxy-5-(methoxy-d3)phenyl)ethan-1,1-d₂-1-amine

Samarium(II) iodide, 0.1 M in THF (80 mL, 8.0 mmol) was placed in an oven-dried flask under Ar. A solution of 2-(2-methoxy-5-(methoxy-d3)phenyl)acetonitrile (240 mg, 1.33 mmol) in anhydrous THE (13 mL) was added followed by Et₃N (4.85 g, 6.7 mL, 47.9 mmol) and D₂O (0.96 g, 0.9 mL, 47.9 mmol) and the mixture stirred vigorously for 15 min. The excess samarium(II) iodide was oxidized by exposing the mixture to air for 20 min, then diluted with DCM (70 mL) and 1M aqueous NaOH (70 mL). The layers were separated and the aqueous layer was extracted with DCM (3×50 mL). The combined organic layers were washed with a 10% aqueous Na₂S₂O₃ solution (2×60 mL), dried (MgSO₄) and concentrated to give an oil. This material was dissolved in 1M hydrochloric acid (15 mL) and washed with DCM (3×15 mL). The aqueous phase was basified to pH ˜11 with 5M aqueous NaOH and extracted with DCM (3×15 mL). The combined organic layers were dried (MgSO₄) and concentrated to give 2-(2-methoxy-5-(methoxy-d₃)phenyl)ethan-1,1-d₂-1-amine (88 mg, 36%) as an oil. LC-MS (+ve mode): Rₜ=3.4 min, m/z=187.15 [M+H]⁺; ¹H NMR (300 MHz, CDCl₃) δ 6.75 (m, 3H, 3×ArH), 3.37 (s, 3H, OCH₃) 2.74 (s, 2H, CH₂), 1.71 (br. s, 2H, NH₂); ¹³C NMR (75.5 MHz, CDCl₃) δ 153.5, 152.1, 144.0, 129.4, 117.1, 111.5, 56.0, 34.7.

### Step 2: Preparation of 2-(4-bromo-2-methoxy-5-(methoxy-d3)phenyl)ethan-1,1-d₂₋₁-amine hydrochloride

To a stirred mixture of 2-(2-methoxy-5-(methoxy-d₃)phenyl)ethan-1,1-d₂-1-amine (85 mg, 0.46 mmol) in AcOH (400 μL) at 5° C. was added a solution of bromine, 1.0 M in AcOH (411 μL, 0.41 mmol) dropwise over 5 min. The =mixture was stirred at 5° C. for 5 min, then diluted with Et₂O (3 mL) and 10% (w/v) aqueous Na₂S₂O₃ (3 mL) and stirred for 30 min. The Et₂O was removed under reduced pressure and the resultant aqueous phase was basified to pH ˜11 with a solution of 15% (w/v) aqueous NaOH. The free base was extracted with DCM (4×15 mL) and the combined organic layers were dried (MgSO₄) and concentrated. The crude residue was stirred in a solution of 1M HCl in Et₂O (7.0 mL, 7.0 mmol) for 30 min. The resulting solid was collected by filtration and the filter cake was washed with Et₂O (4×2 mL) to give impure 2-(4-bromo-2-methoxy-5-(methoxy-d₃)phenyl)ethan-1,1-d₂-1-amine hydrochloride (76 mg) as a solid. This material was purified by reversed-phase chromatography, eluting with 0 to 100% MeOH in 0.02% hydrochloric acid to give 2-(4-bromo-2-methoxy-5-(methoxy-d₃)phenyl)ethan-1,1-d₂-1-amine hydrochloride (31 mg, 25%) as a solid. HPLC: Rₜ=13.8 min; LC-MS (+ve mode): Rₜ=7.3 min, m/z=265.05 and 267.05 [M+H]⁺; ¹H NMR (300 MHz, CD₃OD) δ 7.19 (s, 1H, ArH), 6.97 (s, 1H, ArH), 3.63 (s, 3H, OCH₃), 2.95 (s, 2H, CH₂); ¹³C NMR (75.5 MHz, CD₃OD) δ 153.3, 151.7, 126.2, 117.2, 116.3, 111.4, 56.6, 29.6.

### Example 3: Synthesis of 2-(4-Bromo-2-methoxy-5-(methoxy-d3)phenyl)ethan-1-amine hydrochloride

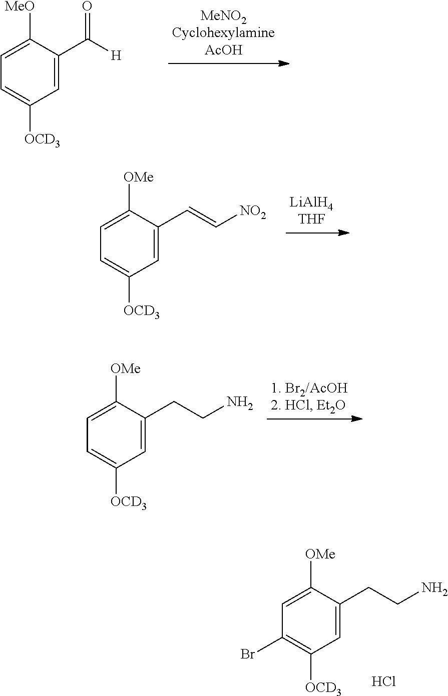

### Step 1: Preparation of (E)-1-methoxy-4-(methoxy-d3)-2-(2-nitrovinyl)benzene

To 2-methoxy-5-(methoxy-d3)benzaldehyde (300 mg, 1.77 mmol) in AcOH (2.0 mL) in a microwave vial was added nitromethane (216 mg, 200 μL, 3.55 mmol) followed by cyclohexylamine (176 mg, 200 μL, 1.77 mmol). The vial was sealed and the mixture was heated to 120° C. under microwave irradiation and stirred for 30 min. After cooling to rt and diluting with H₂O (5 mL), the precipitate was collected by filtration and the filter cake was washed with H₂O (5×5 mL). The solid was dissolved in DCM (20 mL) and washed with satd. NaHCO₃ (3×10 mL), dried (Na₂SO₄) and concentrated to give (E)-1-methoxy-4-(methoxy-d₃)-2-(2-nitrovinyl)benzene (265 mg, 71%) as a solid. HPLC: Rₜ=14.4 min; LC-MS (+ve mode): Rₜ=7.4 min, m/z=213.05 [M+H]⁺; ¹H NMR (300 MHz, CDCl₃) δ 8.05 (d, 1H, J=13.6 Hz, CH), 7.78 (d, 1H, J=13.6 Hz, CH), 6.90 (m, 3H, 3×ArH), 3.84 (s, 3H, CH₃); ¹³C NMR (75.5 MHz, CDCl₃) δ 154.0, 153.5, 138.5, 135.3, 119.5, 119.2, 116.3, 112.4, 56.0.

### Step 2: Preparation of 2-(2-methoxy-5-(methoxy-d3)phenyl)ethan-1-amine

To a stirred solution of LiAlH₄, 2.4 M in THF (1.6 mL, 3.75 mmol) in THF (3 mL) was added a solution of (E)-1-methoxy-4-(methoxy-d₃)-2-(2-nitrovinyl)benzene (265 mg, 1.25 mmol) in THF (6 mL) dropwise. The mixture was heated to reflux and stirred for 4 h before cooling and stirring at rt for 16 h. The mixture was diluted with Et₂O (5 mL), cooled to 0° C. and quenched with H₂O (0.5 mL), 15% aqueous NaOH (0.5 mL) and H₂O (1.5 mL), before warming to rt and stirring for 15 min. Anhydrous MgSO₄ was added and the mixture was stirred for a further 15 min then filtered through Celite and the filtrate was concentrated. The residue was acidified with 1M aqueous HCl (5 mL) and the mixture was extracted with DCM (2×5 mL). The aqueous layer was basified with a 15% aqueous NaOH and extracted with DCM (4×5 mL). The combined organics were dried (MgSO₄) and concentrated to give 2-(2-methoxy-5-(methoxy-d3)phenyl)ethan-1-amine (141 mg, 62%) as an oil. LC-MS (+ve mode): Rₜ=3.6 min, m/z=185.10 [M+H]⁺; ¹H NMR (300 MHz, CDCl₃) δ 6.77 (m, 3H, 3×ArCH), 3.80 (s, 3H, CH₃), 2.95 (t, 2H, J 6.9 Hz, CH₂), 2.77 (t, 2H, J 6.9 Hz, CH₂); ¹³C NMR (75.5 MHz, CDCl₃) δ 153.4, 151.9, 129.3, 116.9, 111.3, 111.3, 55.9, 42.2, 34.8.

### Step 3: Preparation of 2-(4-bromo-2-methoxy-5-(methoxy-d3)phenyl)ethan-1-amine hydrochloride

To a stirred solution of 2-(2-methoxy-5-(methoxy-d₃)phenyl)ethan-1-amine (308 mg, 1.67 mmol) in AcOH (400 μL) was added a solution of bromine (290 mg, 94 μL, 1.83 mmol) in AcOH (200 μL) dropwise and the mixture was stirred at rt for 10 min. The mixture was diluted with Et₂O (8 mL) and a 10% (w/v) aqueous Na₂S₂O₃ (15 mL) was added with subsequent stirring for 15 min. A solid formed and was collected by filtration and the filter cake was washed with Et₂O (3×5 mL) to give a solid after drying. This material was dissolved in MeOH (2 mL) to which 15% (w/v) aqueous NaOH was added to obtain pH ˜11. The free base was extracted with DCM (4×10 mL), and the combined organic layers were dried (MgSO₄) and concentrated to give a solid. The solid was stirred in a solution of 2M HCl in Et₂O (2 mL) for 40 min, then collected by filtration and the filter cake was washed with Et₂O (5×3 mL) to give a solid (184 mg). This material was purified by reversed-phase chromatography, eluting with 0 to 100% MeOH in 0.02% hydrochloric acid to give 2-(4-bromo-2-methoxy-5-(methoxy-d3)phenyl)ethan-1-amine hydrochloride (106 mg, 21%) as a solid. HPLC: Rₜ=13.5 min; LC-MS (+ve mode): Rₜ=7.3 min, m/z=263.00 and 265. 05 [M+H]⁺; ¹H NMR (300 MHz, DMSO-d₆) δ 8.02 (s, 3H, NH₃), 7.21 (s, 1H, ArH), 7.01 (s, 1H, ArH), 3.77 (s, 3H, CH₃), 2.98 (m, 2H, CH₂), 2.85 (m, 2H, CH₂); ¹³C NMR (DMSO-d₆) δ 152.0, 149.8, 126.0, 116.3, 115.6, 109.3, 56.7, 38.8, 28.3.

### Example 4: Synthesis of 2-(4-bromo-5-methoxy-2-(methoxy-d3)phenyl)ethan-1,1,2,2-d₄-1-amine hydrochloride

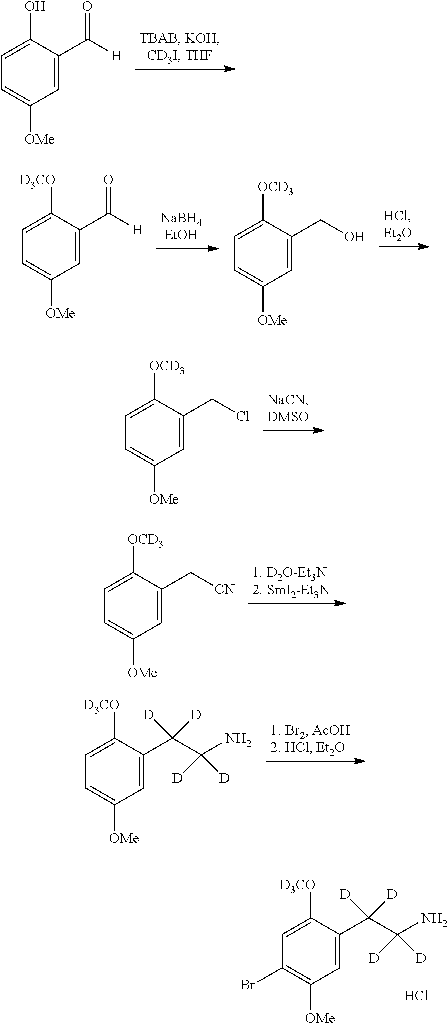

### Step 1: Preparation of 5-methoxy-2-(methoxy-d3)benzaldehyde

To 2-hydroxy-5-methoxybenzaldehyde (1.00 g, 6.6 mmol), tetra-n-butylammonium bromide (84 mg, 0.26 mmol) and ground KOH (0.92 g, 16.4 mmol) under an atmosphere of N₂ was added iodomethane-d₃ (2.95 g, 1.3 mL, 20.4 mmol) slowly with stirring and the reaction mixture was heated to 40° C. and stirred for 24 h. The mixture was diluted with H₂O (20 mL) and extracted with Et₂O (4×15 mL). The combined organic layers were washed with satd. brine (20 mL), dried (MgSO₄) and concentrated to give 5-methoxy-2-(methoxy-d3)benzaldehyde (1.16 g, quant.) as a solid. TLC: R_{f}=0.55 (ethyl acetate-petrol, 4:6 v/v); ¹H NMR (300 MHz, CDCl₃) δ 10.38 (s, 1H, CH), 7.27 (d, 1H, J=3.2 Hz, ArH), 7.07 (dd, 1H, J=9.0, 3.2 Hz, ArH), 6.87 (d, 1H, J=9.0 Hz, ArH), 3.73 (s, 3H, OCH₃); ¹³C NMR (75.5 MHz, CDCl₃) δ 189.6, 156.7, 153.6, 124.9, 123.5, 113.3, 110.4, 55.8.

### Step 2: Synthesis of (5-methoxy-2-(methoxy-d3)phenyl)methanol

To 5-methoxy-2-(methoxy-d3)benzaldehyde (500 mg, 2.96 mmol) in anhydrous EtOH (60 mL) under an atmosphere of N₂ was added NaBH₄ (168 mg, 4.43 mmol) and the mixture was heated to 80° C. and stirred for 2 h. The mixture was cooled to rt, H₂O (60 mL) was added and the EtOH was removed in vacuo and the residue was extracted with EtOAc (4×30 mL). The combined organic layers were washed with satd. brine (20 mL), dried (MgSO₄) and concentrated to give (5-methoxy-2-(methoxy-d3)phenyl)methanol (419 mg, 83%) as an oil. TLC: R_{f}=0.41 (ethyl acetate-petrol, 3:7 v/v); ¹H NMR (300 MHz, CDCl₃) δ 6.81 (m, 1H, ArH), 6.73 (m, 2H, 2×ArH), 4.59 (d, 2H, J=6.3 Hz, CH₂), 3.71 (s, 3H, OCH₃), 2.28 (t, 1H, J 6.5 Hz, OH); ¹³C NMR (75.5 MHz, CDCl₃) δ 153.6, 151.5, 130.1, 114.8, 113.0, 111.1, 62.2, 55.8.

### Step 3: Synthesis of 2-(chloromethyl)-4-methoxy-l-(methoxy-d3)benzene

To (5-methoxy-2-(methoxy-d3)phenyl)methanol (419 g, 2.45 mmol) in Et₂O (2.5 mL) was added hydrochloric acid, 12M (2.5 mL) and the mixture was stirred at rt for 3 h. Upon completion, the mixture was diluted with H₂O (15 mL) and extracted with Et₂O (3×15 mL). The combined organic layers were washed with satd. brine (15 mL), dried (MgSO₄) and concentrated to give 2-(chloromethyl)-4-methoxy-1-(methoxy-d3)benzene (465 mg, quant.) as a solid. TLC: R_{f}=0.66 (ethyl acetate-petrol, 3:7 v/v); ¹H NMR (300 MHz, CDCl₃) δ 6.87 (m, 1H, ArH), 6.76 (m, 2H, 2×ArH), 4.56 (s, 2H, CH₂), 3.71 (s, 3H, OCH₃); ¹³C NMR (75.5 MHz, CDCl₃) δ 153.5, 151.5, 126.7, 116.2, 114.7, 112.0, 55.8, 41.6.

### Step 4: Preparation of 2-(5-methoxy-2-(methoxy-d3)phenyl)acetonitrile

To a suspension of NaCN (180 mg, 3.68 mmol) in anhydrous DMSO (1.5 mL) at rt under an atmosphere of N₂ was added a solution of 2-(chloromethyl)-1-methoxy-4-(methoxy-d₃)benzene (465 mg, 2.45 mmol) in anhydrous DMSO (3.0 mL). The mixture was heated to 80° C. and stirred for 2 h, then cooled, quenched with H₂O (30 mL) and extracted with EtOAc (3×20 mL). The combined organic layers were washed with H₂O (3×20 mL), satd. brine (20 mL), dried (MgSO₄) and concentrated to give a semi-solid (388 mg). This material was purified by column chromatography on silica gel, eluting with 7 to 60% EtOAc in petrol (40:60) to give 2-(5-methoxy-2-(methoxy-d3)phenyl)acetonitrile (281 mg) as a solid. TLC: R_{f}=0.40 (ethyl acetate-petrol, 3:7 v/v); ¹H NMR (300 MHz, CDCl₃) δ 6.88 (m, 1H, ArH), 6.75 (m, 2H, 2×ArH), 3.71 (s, 3H, OCH₃), 3.60 (s, 2H, CH₂); ¹³C NMR (75.5 MHz, CDCl₃) δ 153.6, 119.4, 117.9, 115.4, 113.9, 111.4, 55.9, 18.8.

### Step 5: Preparation of 2-(5-methoxy-2-(methoxy-d3)phenyl)ethan-1,1,2,2-d₄-1-amine

A solution of 2-(5-methoxy-2-(methoxy-d3)phenyl)acetonitrile (198 mg, 1.10 mmol) in anhydrous THF (1.0 mL) was placed under an atmosphere of Ar. Et₃N (1.35 g, 1.8 mL, 13.3 mmol) and D₂O (2.66 g, 2.4 mL, 133 mmol) were added at rt and the mixture was stirred vigorously for 16 h. After this time, Et₃N (1.35 g, 1.8 mL, 13.3 mmol) and samarium(II) iodide solution, 0.1M in THF (100 mL, 10.0 mmol) were added at rt. After 30 min the excess samarium(II) iodide was oxidized by opening the mixture to air and stirring for 20 min. The mixture was diluted with DCM (60 mL) and 1M aqueous NaOH (60 mL), then separated and the aqueous phase was extracted with DCM (3×50 mL). The combined organic layers were washed with satd. aqueous Na₂S₂O₃ (2×100 mL), dried (MgSO₄) and concentrated to give an oil. This material was dissolved in 1M hydrochloric acid (5 mL) and washed with DCM (2×5 mL). The aqueous phase was basified to pH ˜11 with 15% (w/v) aqueous NaOH and extracted with DCM (4×5 mL). The combined organic layers were dried (MgSO₄) and concentrated to give 2-(5-methoxy-2-(methoxy-d₃)phenyl)ethan-1,1,2,2-d₄-1-amine (97 mg, 47%) as an oil. HPLC: Rₜ=5.1 min; LC-MS (+ve mode): Rₜ=3.3 min, m/z=189.15 [M+H]⁺; ¹H NMR (300 MHz, CDCl₃) δ 6.67 (m, 3H, 3×ArCH), 3.69 (s, 3H, OCH₃); ¹³C NMR (75.5 MHz, CDCl₃) δ 153.4, 151.9, 129.0, 116.9, 111.4, 111.3, 55.7.

### Step 6: Preparation of 2-(4-bromo-5-methoxy-2-(methoxy-d3)phenyl)ethan-1,1,2,2-d₄-1-amine hydrochloride

To a stirred mixture of 2-(5-methoxy-2-(methoxy-d₃)phenyl)ethan-1,1,2,2-d₄-1-amine (95 mg, 0.50 mmol) in AcOH (200 μL) was added dropwise a solution of bromine (89 mg, 28 μL, 0.56 mmol) in AcOH (100 μL). The mixture was stirred at rt for 10 min, then diluted with Et₂O (5 mL) followed by 10% (w/v) aqueous Na₂S₂O₃ (5 mL) and stirred for 30 min. A solution of 15% (w/v) aqueous NaOH was added to obtain pH ˜11, then extracted with DCM (4×5 mL) and the combined organic layers were dried (MgSO₄) and concentrated to give an oil. The residue was stirred in a solution of 2M HCl in Et₂O (1.5 mL, 3.0 mmol) for 40 min and resulting solid was collected by filtration and the filter cake was washed with Et₂O (5×3 mL) to give a solid (90 mg). This material was purified by reversed-phase chromatography, eluting with 0 to 100% MeOH in 0.02% hydrochloric acid to give 2-(4-bromo-5-methoxy-2-(methoxy-d₃)phenyl)ethan-1,1,2,2-d₄-1-amine hydrochloride (29 mg, 19%) as a solid. HPLC: Rₜ=12.1 min; LC-MS (+ve mode): Rₜ=7.3 min, m/z=267.05 and 269.05 [M+H]⁺; ¹H NMR (300 MHz, DMSO-d₆) δ 7.96 (br. s, 3H, NH₃⁺), 7.21 (s, 1H, ArH), 7.01 (s, 1H, ArH), 3.84 (s, 3H, OCH₃); ¹³C NMR (75.5 MHz, DMSO-d₆) δ 152.0, 149.8, 125.9, 116.3, 115.6, 109.3, 57.1.

### Example 5: Synthesis of 2-(4-bromo-5-methoxy-2-(methoxy-d3)phenyl)ethan-1-amine hydrochloride

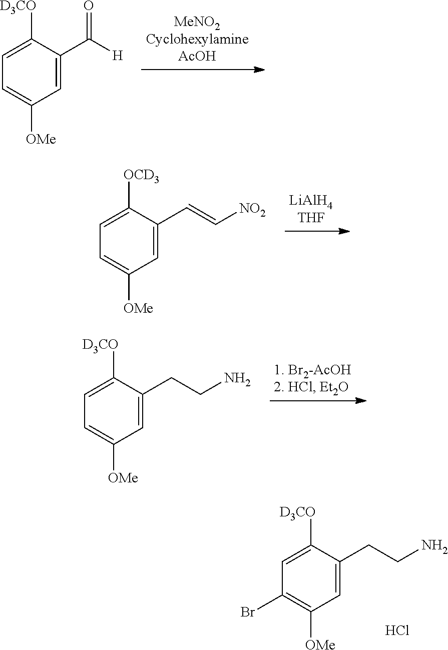

### Step 1: Preparation of (E)-4-methoxy-l-(methoxy-d3)-2-(2-nitrovinyl)benzene

To a mixture of 5-methoxy-2-(methoxy-d3)benzaldehyde (200 mg, 1.18 mmol) in AcOH (1.3 mL) in a microwave vial was added nitromethane (144 mg, 130 μL, 2.36 mmol) followed by cyclohexylamine (117 mg, 140 μL, 1.18 mmol). The vial was sealed and heated to 120° C. under microwave irradiation and stirred for 30 min. The mixture was cooled to rt and diluted with H₂O (5 mL). The emerging precipitate was collected by filtration and the filter cake was washed with H₂O (5×5 mL), then dissolved in DCM (20 mL) and washed with satd. aqueous NaHCO₃ (3×8 mL), dried (Na₂SO₄) and concentrated to give (E)-4-methoxy-l-(methoxy-d3)-2-(2-nitrovinyl)benzene (195 mg, 78%) as a solid. HPLC: Rₜ=14.4 min; LC-MS (+ve mode): Rₜ=7.7 min, m/z=213.05 [M+H]⁺; ¹H NMR (300 MHz, CDCl₃) δ 8.05 (d, 1H, J=13.6 Hz, CH), 7.79 (d, 1H, J=13.6 Hz, CH), 6.95 (m, 3H, 3 xArH), 3.73 (s, 3H, CH₃); ¹³C NMR (75.5 MHz, CDCl₃) δ 154.0, 153.5, 138.5, 135.3, 119.5, 119.2, 116.3, 112.4, 55.9.

### Step 3: Preparation of 2-(5-methoxy-2-(methoxy-d3)phenyl)ethan-1-amine

To a stirred solution of LiAlH₄, 2.4 M in THF (1.15 mL, 2.76 mmol) in THF (3 mL) was added dropwise a solution of (E)-4-methoxy-1-(methoxy-d₃)-2-(2-nitrovinyl)benzene (195 mg, 0.92 mmol) in THF (3.6 mL). The mixture was heated to reflux and stirred for 4 h before cooling and stirring at rt for 16 h. The mixture was diluted with Et₂O (4 mL), cooled to 0° C. and quenched with H₂O (0.3 mL), 15% (w/v) aqueous NaOH (0.3 mL) and H₂O (0.9 mL), before warming to rt and stirring for 15 min. Anhydrous MgSO₄ was added and the mixture was stirred for a further 15 min then filtered through Celite and the filtrate was concentrated to give 2-(5-methoxy-2-(methoxy-d3)phenyl)ethan-1-amine (168 mg, 99%) as an oil. HPLC: Rₜ=2.5 min; LC-MS (+ve mode): Rₜ=1.4 min, m/z=185.10 [M+H]⁺; ¹H NMR (300 MHz, CDCl₃) δ 6.67 (m, 3H, 3×ArH), 3.69 (s, 3H, CH₃), 2.85 (t, 2H, J=6.9 Hz, CH₂), 2.66 (t, 2H, J=6.9 Hz, CH₂); ¹³C NMR (75.5 MHz, CDCl₃) δ 153.4, 152.0, 129.4, 116.9, 111.3 (2×C), 55.7, 42.3, 35.0.

### Step 4: Preparation of 2-(4-bromo-5-methoxy-2-(methoxy-d3)phenyl)ethan-1-amine hydrochloride

To a stirred mixture of 2-(5-methoxy-2-(methoxy-d₃)phenyl)ethan-1-amine (168 mg, 0.91 mmol) in AcOH (300 μL) was added dropwise a solution of bromine (163 mg, 50 μL, 1.02 mmol) in AcOH (300 μL) and the resulting mixture was stirred at rt for 30 min. The mixture was diluted with Et₂O (3 mL) and 10% (w/v) aqueous Na₂S₂O₃ (10 mL) and stirred for 5 min. A solid formed that was collected by filtration and the filter cake was washed with Et₂O (3×3 mL). This material was dissolved in MeOH (2 mL) to which 15% (w/v) aqueous NaOH was added to obtain pH ˜11 that was extracted with DCM (3×3 mL) and the combined organic layers were concentrated to give a solid. The solid was stirred in a solution of HCl in Et₂O (2 M, 2 mL) for 30 min, then collected by filtration and the filter cake was washed with Et₂O (5×3 mL) to give 2-(4-bromo-5-methoxy-2-(methoxy-d3)phenyl)ethan-1-amine hydrochloride (80 mg, 32%) as a solid. HPLC: Rₜ=6.4 min; LC-MS (+ve mode): Rₜ=2.8 min, m/z=263.05 and 265. 05 [M+H]⁺; ¹H NMR (300 MHz, DMSO-d₆) δ 7.87 (br. s, 3H, NH₃⁺), 7.21 (s, 1H, ArH), 7.00 (s, 1H, ArH), 3.80 (s, 3H, CH₃), 2.99 (m, 2H, CH₂), 2.84 (m, 2H, CH₂); ¹³C NMR (75.5 MHz, DMSO-d₆) δ 152.0, 149.8, 125.9, 116.3, 115.6, 109.4, 57.1, 38.8, 28.4.

### Synthesis of 2-(4-bromo-5-methoxy-2-(methoxy-d3)phenyl)ethan-1,1-d₂-1-amine hydrochloride

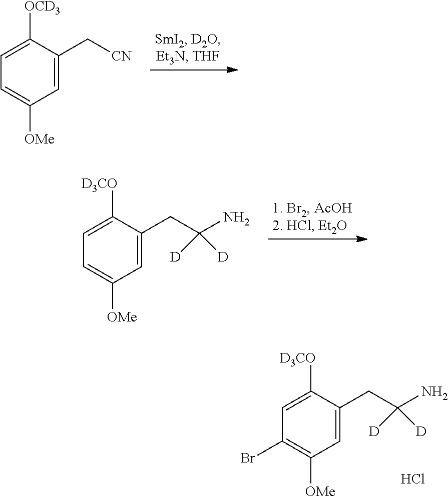

### Step 1: Preparation of 2-(5-Methoxy-2-(methoxy-d3)phenyl)ethan-1,1-d₂-1-amine

Samarium(II) iodide, 0.1 M in THF (80 mL, 8.0 mmol) was placed in an oven-dried flask under an atmosphere of Ar. A solution of 2-(5-methoxy-2-(methoxy-d3)phenyl)acetonitrile (300 mg, 1.66 mmol) in anhydrous THF (16 mL) was added followed by Et₃N (6.05 g, 8.3 mL, 59.9 mmol) and D₂O (1.20 g, 1.1 mL, 59.9 mmol) and the mixture stirred vigorously for 15 min. The excess samarium(II) iodide was oxidized by opening the mixture to air and stirring for 20 min. The mixture was diluted with DCM (90 mL) and 1M aqueous NaOH (90 mL) and the layers were separated. The aqueous phase was extracted with DCM (3×60 mL) and the combined organic layers were washed with a 10% aqueous Na₂S₂O₃ solution (2×80 mL), dried (MgSO₄) and concentrated to give an oil. This material was dissolved in 1M hydrochloric acid (15 mL) and washed with DCM (3×15 mL). The aqueous phase was basified to pH ˜11 with 5M aqueous NaOH and extracted with DCM (3×15 mL). The combined organic layers were dried (MgSO₄) and concentrated to give 2-(5-methoxy-2-(methoxy-d₃)phenyl)ethan-1,1-d₂-1-amine (77 mg, 25%) as an oil. LC-MS (+ve mode): Rₜ=3.4 min, m/z=187.15 [M+H]⁺; ¹H NMR (300 MHz, CDCl₃) δ 6.76 (m, 3H, 3×ArH), 3.76 (s, 3H, CH₃) 2.70 (s, 2H, CH₂), 1.95 (br. s, 2H, NH₂); ¹³C NMR (75.5 MHz, CDCl₃) 153.6, 152.0, 128.5, 117.2, 111.8, 111.4, 55.9, 33.7.

### Step 2: Preparation of 2-(4-Bromo-5-methoxy-2-(methoxy-d3)phenyl)ethan-1,1-d₂-1-amine hydrochloride

To a stirred mixture of 2-(5-methoxy-2-(methoxy-d₃)phenyl)ethan-1,1-d₂-1-amine (77 mg, 0.41 mmol) in AcOH (140 μL) at 0° C. was added dropwise a solution of bromine (1.0 M in AcOH, 370 μL, 0.37 mmol). After addition of approximately 150 μL of the bromine solution, stirring ceased and a further portion of AcOH (200 μL) was added until stirring resumed. A further 50 μL of the bromine solution was added at 0° C. and stirring ceased. The reaction vessel was removed from the ice bath and the remaining bromine solution was added at rt. The mixture was stirred at rt for 5 min, then diluted with Et₂O (10 mL) and 10% (w/v) aqueous Na₂S₂O₃ (10 mL) and stirred for 20 min. The Et₂O was removed under reduced pressure and the resultant aqueous phase was basified to pH ˜11 with a solution of 15% (w/v) aqueous NaOH. The free base was extracted with DCM (4×15 mL) and the combined organic layers were dried (MgSO₄) and concentrated. The residue was stirred in a solution of 2M HCl in Et₂O (3.5 mL, 7.0 mmol) for 30 min, and the emerging solid was collected by filtration and the filter cake was washed with Et₂O (4×3 mL) to give impure 2-(4-bromo-5-methoxy-2-(methoxy-d₃)phenyl)ethan-1,1-d₂-1-amine hydrochloride (70 mg) as a solid. This material was purified by reversed-phase chromatography, eluting with 0 to 100% MeOH in 0.02% hydrochloric acid to give 2-(4-bromo-5-methoxy-2-(methoxy-d₃)phenyl)ethan-1,1-d₂-1-amine hydrochloride (23 mg, 21%) as a solid. HPLC: Rₜ=13.8 min; LC-MS (+ve mode): Rₜ=7.3 min, m/z=265.05 and 267.05 [M+H]⁺; ¹H NMR: (300 MHz, CD₃OD) δ 7.18 (s, 1H, ArH), 6.95 (s, 1H, ArH), 3.84 (s, 3H, OCH₃), 2.94 (s, 2H, CH₂); ¹³C NMR (75.5 MHz, CD₃OD) δ 153.4, 151.7, 126.1, 117.2, 116.3, 111.5, 57.4, 29.6.

### Example 6: Evaluation of Metabolic Stability of Deuterated 2C-B in Human Liver Microsomes

### Materials and Protocol

Pooled human liver microsomes were purchased from a commercial supplier. Microsomes were stored at −80° C. prior to use. Microsomes (final protein concentration 0.5 mg/mL), 0.1 M phosphate buffer pH 7.4 and the test compound (final substrate concentration 1 μM; final DMSO concentration 0.25%) were pre-incubated at 37° C. prior to addition of NADPH (final concentration 1 mM) to initiate the reaction. A minus cofactor control incubation was included for each test compound where 0.1 M phosphate buffer pH 7.4 was added instead of NADPH (minus NADPH). Two control compounds (dextromethorphan and verapamil) were included with each test compound. All incubations were performed individually for each test compound. Each test compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates were then centrifuged at 3,000 rpm for 20 min at 4° C. to precipitate the protein.

### Data Analysis

From a plot of ln peak area ratio (test compound peak area/internal standard peak area) against time, the gradient of the line was determined. Subsequently, half-life and intrinsic clearance of each test compound was calculated using the equations below:

Elimination rate constant (k)=(−gradient)

Half-life (t½)(min)=0.693/k

Intrinsic clearance (CLᵢₙₜ)(μL/min/mg protein)=V×0.693/t1/2

where V=Incubation volume (μL)/Microsomal protein (mg).

Relevant control compounds were assessed, ensuring intrinsic clearance values fall within the specified limits (if available).

### Results

The calculated half-life and intrinsic clearance of each test compound in human liver microsomes are summarized in Table 3.

TABLE 3

| | CLᵢₙₜ (μL/min/mg | SE | | |
|---|---|---|---|---|
| Compound | protein) | CLᵢₙₜ | t_{1/2} (min) | Timepoints |
| 2C-B HCl salt | 9.56 | 1.56 | 145 | 5 |
| 2-(4-bromo-2-methoxy-5-(methoxy- | 10.5 | 3.46 | 132 | 5 |
| d₃)phenyl)ethan-1,1,2,2-d₄-1-amine HCl salt | | | | |
| 2-(4-bromo-2-methoxy-5-(methoxy- | 10.9 | 3.35 | 127 | 5 |
| d₃)phenyl)ethan-1,1-d₂-1-amine HCl salt | | | | |
| 2-(4-Bromo-2-methoxy-5-(methoxy- | 9.22 | 1.24 | 150 | 5 |
| d₃)phenyl)ethan-1-amine HCl salt | | | | |
| 2-(4-bromo-5-methoxy-2-(methoxy- | 11.9 | 5.88 | 117 | 5 |
| d₃)phenyl)ethan-1,1,2,2-d₄-1-amine HCl salt | | | | |
| 2-(4-bromo-5-methoxy-2-(methoxy- | 18.2 | 6.21 | 76.3 | 5 |
| d₃)phenyl)ethan-1-amine HCl salt | | | | |
| 2-(4-bromo-5-methoxy-2-(methoxy- | 12.4 | 4.41 | 112 | 5 |
| d₃)phenyl)ethan-1,1-d₂-1-amine HCl salt | | | | |
| dextromethorphan | 53.0 | 4.58 | 26.2 | 5 |
| verapamil | 365 | 11.2 | 3.80 | 3 |
| dextromethorphan | 51.2 | 0.908 | 27.1 | 5 |
| verapamil | 309 | 10.1 | 4.49 | 3 |
| dextromethorphan | 55.0 | 3.00 | 25.2 | 5 |
| verapamil | 310 | 17.1 | 4.47 | 3 |

What is claimed is:

1. A compound of Formula (I), or a stereoisomer or a pharmaceutically acceptable salt thereof: 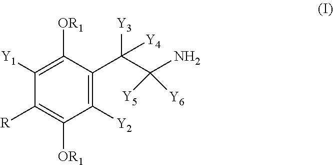
wherein
R is H, D, F, Cl, Br, I, Me, Et, ⁿPr, ⁱPr, ^{c}Pr, ⁿBu, Ph, CH₂Ph, C≡CH (ethynyl), CH═CH₂ (vinyl), CH₂CH═CH₂ (allyl), CH₂OMe, CH₂CF₃, CH₂CH₂F, CHF₂, CF₃, NO₂, NH₂, CN, SeCH₃, OCH₃, OCH(CH₃)₂, SCH₃, SCH₂CH₃, SCH₂C(CH₃)═CH₂, SCH(CH₃)₂, SCH₂CH₂CH₃, SCH₂^{c}Pr, SC(CH₃)₃, SCH₂CH₂OCH₃, SCH₂CH₂SCH₃, S^{c}Pr, SCH₂CH═CH₂, SCH(CH₃)(CH₂CH₃), SCH₂CH₂CH₂CH₃, SCH₂CH₂F, SCH₂CHF₂, SCH₂CF₃, SCH₂CH(CH₃)₂, SCH₂Ph, SCH₂CH₂CH₂F, or SCH₂CH₂CH₂CH₂F;
each R₁ is independently selected from CH₃, CH₂D, CHD₂, and CD₃;
Y₁, Y₂, Y₃, Y₄, Y₅, and Y₆ are each independently H or D; and
at least one R₁ comprises one or more deuterium, or at least one of Y₁, Y₂, Y₃, Y₄, Y₅, and Y₆ is deuterium.

2. The compound or stereoisomer or a pharmaceutically acceptable salt of claim 1, wherein R is H or D.

3. The compound or stereoisomer or a pharmaceutically acceptable salt of claim 1, wherein R is F.

4. The compound or stereoisomer or a pharmaceutically acceptable salt of claim 1, wherein R is Cl.

5. The compound or stereoisomer or a pharmaceutically acceptable salt of claim 1, wherein R is Br.

6. The compound or stereoisomer or a pharmaceutically acceptable salt of claim 1, wherein R is I.

7. The compound or stereoisomer or a pharmaceutically acceptable salt of claim 1, wherein R is Me.

8. The compound or stereoisomer or a pharmaceutically acceptable salt of claim 1, wherein R is Et.

9. The compound or stereoisomer or a pharmaceutically acceptable salt of claim 1, wherein R is ⁿPr.

10. The compound or stereoisomer or a pharmaceutically acceptable salt of claim 1, wherein R is ⁱPr.

11. The compound or stereoisomer or a pharmaceutically acceptable salt of claim 1, wherein R is ^{c}Pr.

12. The compound or stereoisomer or a pharmaceutically acceptable salt of claim 1, wherein R is ⁿBu.

13. The compound or stereoisomer or a pharmaceutically acceptable salt of claim 1, wherein R is Ph.

14. The compound or stereoisomer or a pharmaceutically acceptable salt of claim 1, wherein R is CH₂Ph.

15. The compound or stereoisomer or a pharmaceutically acceptable salt of claim 1, wherein R is C≡CH.

16. The compound or stereoisomer or a pharmaceutically acceptable salt of claim 1, wherein R is CH═CH₂.

17. The compound or stereoisomer or a pharmaceutically acceptable salt of claim 1, wherein R is CH₂CH═CH₂.

18. The compound or stereoisomer or a pharmaceutically acceptable salt of claim 1, wherein R is CH₂OMe.

19. The compound or stereoisomer or a pharmaceutically acceptable salt of claim 1, wherein R is CH₂CF₃.

20. The compound or stereoisomer or a pharmaceutically acceptable salt of claim 1, wherein R is CH₂CH₂F.

21. The compound or stereoisomer or a pharmaceutically acceptable salt of claim 1, wherein R is CHF₂.

22. The compound or stereoisomer or a pharmaceutically acceptable salt of claim 1, wherein R is CF₃.

23. The compound or stereoisomer or a pharmaceutically acceptable salt of claim 1, wherein R i s NO₂.

24. The compound or stereoisomer or a pharmaceutically acceptable salt of claim 1, wherein R is NH₂.

25. The compound or stereoisomer or a pharmaceutically acceptable salt of claim 1, wherein R is CN.

26. The compound or stereoisomer or a pharmaceutically acceptable salt of claim 1, wherein R is SeCH₃.

27. The compound or stereoisomer or a pharmaceutically acceptable salt of claim 1, wherein R is OCH₃.

28. The compound or stereoisomer or a pharmaceutically acceptable salt of claim 1, wherein R is OCH(CH₃)₂.

29. The compound or stereoisomer or a pharmaceutically acceptable salt of claim 1, wherein R is SCH₃.

30. The compound or stereoisomer or a pharmaceutically acceptable salt of claim 1, wherein R is SCH₂CH₃.

31. The compound or stereoisomer or a pharmaceutically acceptable salt of claim 1, wherein R is SCH₂C(CH₃)═CH₂.

32. The compound or stereoisomer or a pharmaceutically acceptable salt of claim 1, wherein R is SCH(CH₃)₂.

33. The compound or stereoisomer or a pharmaceutically acceptable salt of claim 1, wherein R is SCH₂CH₂CH₃.

34. The compound or stereoisomer or a pharmaceutically acceptable salt of claim 1, wherein R is SCH₂^{c}Pr.

35. The compound or stereoisomer or a pharmaceutically acceptable salt of claim 1, wherein R is SC(CH₃)₃.

36. The compound or stereoisomer or a pharmaceutically acceptable salt of claim 1, wherein R is SCH₂CH₂OCH₃.

37. The compound or stereoisomer or a pharmaceutically acceptable salt of claim 1, wherein R is SCH₂CH₂SCH₃.

38. The compound or stereoisomer or a pharmaceutically acceptable salt of claim 1, wherein R is S^{c}Pr.

39. The compound or stereoisomer or a pharmaceutically acceptable salt of claim 1, wherein R is SCH₂CH═CH₂.

40. The compound or stereoisomer or a pharmaceutically acceptable salt of claim 1, wherein R is SCH(CH₃)(CH₂CH₃).

41. The compound or stereoisomer or a pharmaceutically acceptable salt of claim 1, wherein R is SCH₂CH₂CH₂CH₃.

42. The compound or stereoisomer or a pharmaceutically acceptable salt of claim 1, wherein R is SCH₂CH₂F.

43. The compound or stereoisomer or a pharmaceutically acceptable salt of claim 1, wherein R is SCH₂CHF₂.

44. The compound or stereoisomer or a pharmaceutically acceptable salt of claim 1, wherein R is SCH₂CF₃.

45. The compound or stereoisomer or a pharmaceutically acceptable salt of claim 1, wherein R is SCH₂CH(CH₃)₂.

46. The compound or stereoisomer or a pharmaceutically acceptable salt of claim 1, wherein R is SCH₂Ph.

47. The compound or stereoisomer or a pharmaceutically acceptable salt of claim 1, wherein R is SCH₂CH₂CH₂F.

48. The compound or stereoisomer or a pharmaceutically acceptable salt of claim 1, wherein R is SCH₂CH₂CH₂CH₂F.

49. The compound or stereoisomer or a pharmaceutically acceptable salt of claim 1, wherein the compound of Formula (I) has a formula of: 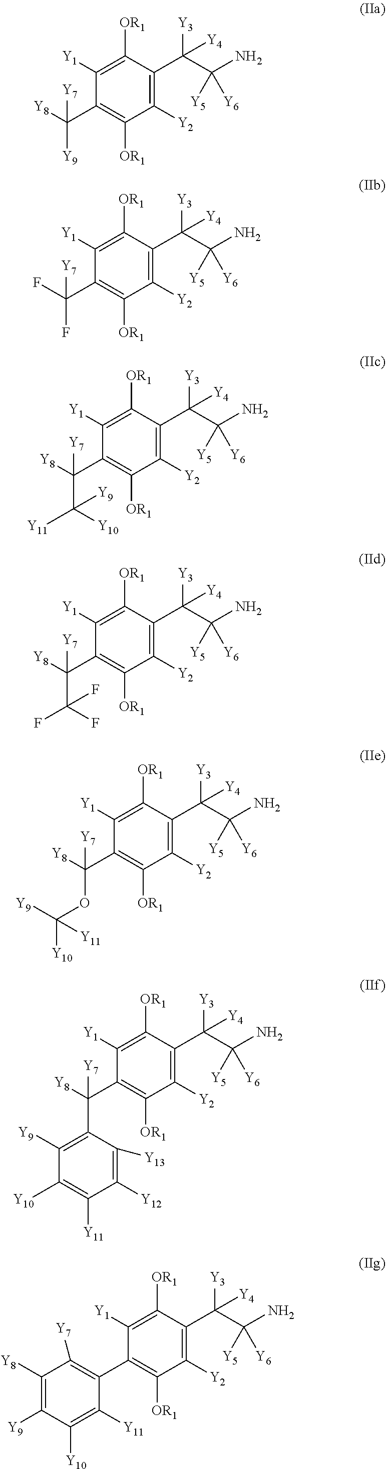 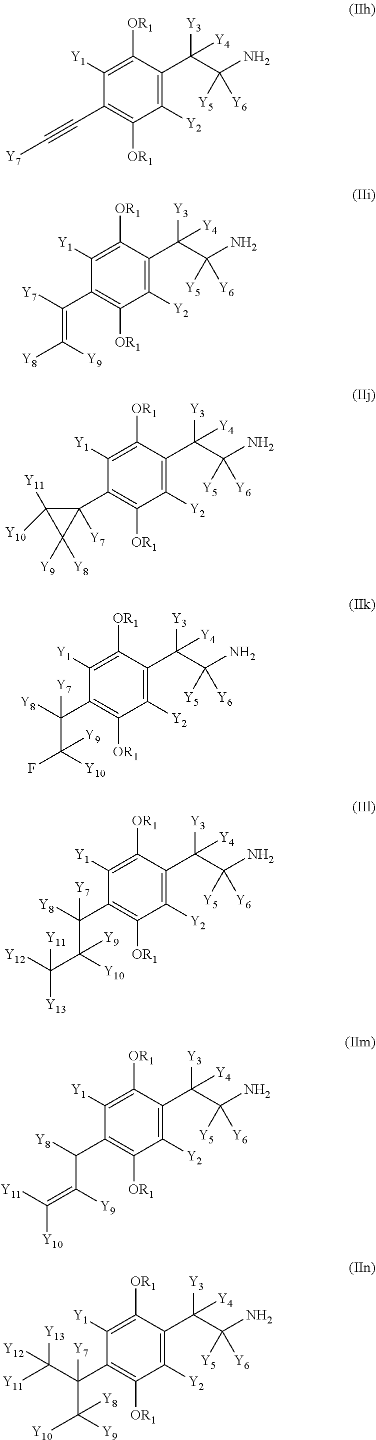 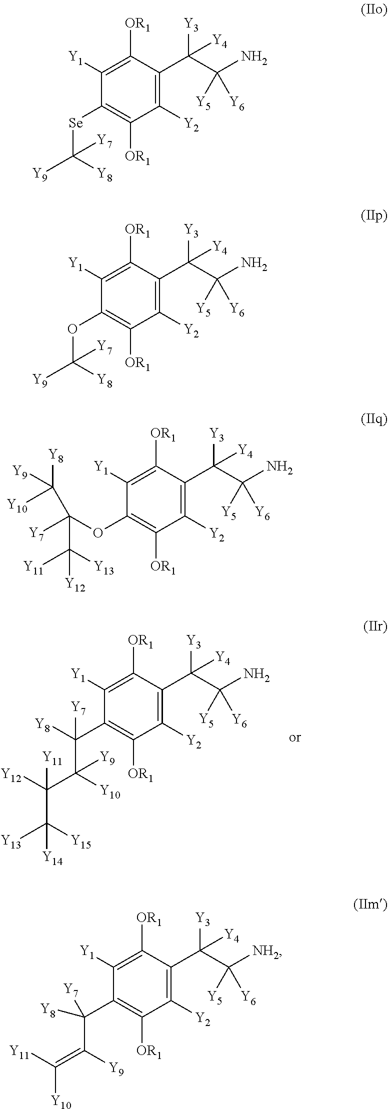
wherein
each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or deuterium; and
wherein at least one R₁ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium.

50. The compound or stereoisomer or a pharmaceutically acceptable salt of claim 1, wherein the compound of Formula (I) has a structure of Formula (II): 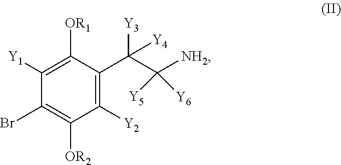
wherein
R₁ and R₂ are each independently selected from CH₃, CH₂D, CHD₂, and CD₃;
Y₁, Y₂, Y₃, Y₄, Y₅, and Y₆ are each independently H or deuterium; and
wherein at least one of R₁ and R₂ comprises one or more deuterium, or at least one of Y₁, Y₂, Y₃, Y₄, Y₅, and Y₆ is deuterium.

51. The compound or stereoisomer or a pharmaceutically acceptable salt of claim 50,
wherein the compound of Formula (II) has a formula of Formula (II-A): 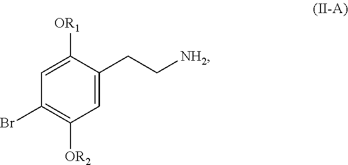
wherein at least one of R₁ and R₂ comprises one or more deuterium.

52. The compound or stereoisomer or a pharmaceutically acceptable salt of claim 50, wherein the compound of Formula (II) has a formula of Formula (II-B): 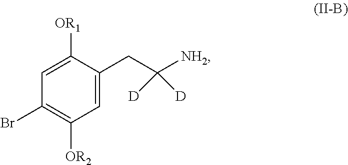
wherein R₁ and R₂ are each independently selected from CH₃, CH₂D, CHD₂, and CD₃.

53. The compound or stereoisomer or a pharmaceutically acceptable salt of claim 50, wherein the compound of Formula (II) has a formula of Formula (II-C): 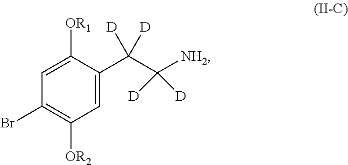
wherein R₁ and R₂ are each independently selected from CH₃, CH₂D, CHD₂, and CD₃.

54. The compound or stereoisomer or a pharmaceutically acceptable salt of claim 50, wherein Y₅ and Y₆ are each deuterium, and Y₁, Y₂, Y₃, and Y₄ are each hydrogen.

55. The compound or stereoisomer or a pharmaceutically acceptable salt of claim 50, wherein Y₅ is deuterium, and Y₁, Y₂, Y₃, Y₄, and Y₆, are each hydrogen.

56. The compound or stereoisomer or a pharmaceutically acceptable salt of claim 50, wherein Y₃, Y₄, Y₅, and Y₆ are each deuterium, and Y₁ and Y₂ are each hydrogen

57. The compound or stereoisomer or a pharmaceutically acceptable salt of claim 50, wherein Y₃ and Y₆ are each deuterium, and Y₁, Y₂, Y₄, and Y₅ are each hydrogen.

58. The compound or stereoisomer or a pharmaceutically acceptable salt of claim 50, wherein Y₃ and Y₄ are each deuterium, and Y₁, Y₂, Y₅, and Y₆ are each hydrogen.

59. The compound or stereoisomer or a pharmaceutically acceptable salt of claim 50, wherein Y₃ is deuterium, and Y₁, Y₂, Y₄, Y₅, and Y₆ are each hydrogen.

60. The compound or stereoisomer or a pharmaceutically acceptable salt of claim 50, wherein Y₁, Y₂, Y₃, Y₄, Y₅, and Y₆ are each deuterium.

61. The compound or stereoisomer or a pharmaceutically acceptable salt of claim 50, wherein Y₁, Y₂, Y₅, and Y₆ are each deuterium, and Y₃ and Y₄ are each hydrogen.

62. The compound or stereoisomer or a pharmaceutically acceptable salt of any one of claims 50-58, wherein R₁ is CD₃.

63. The compound or stereoisomer or a pharmaceutically acceptable salt of any one of claims 50-58, wherein R₁ is CHD₂.

64. The compound or stereoisomer or a pharmaceutically acceptable salt of any one of claims 50-58, wherein R₁ is CH₂D.

65. The compound or stereoisomer or a pharmaceutically acceptable salt of any one of claims 50-58, wherein R₁ is CH₃.

66. The compound or stereoisomer or a pharmaceutically acceptable salt of any one of claims 50-65, wherein R₂ is CD₃.

67. The compound or stereoisomer or a pharmaceutically acceptable salt of any one of claims 50-65, wherein R₂ is CHD₂.

68. The compound or stereoisomer or a pharmaceutically acceptable salt of any one of claims 50-65, wherein R₂ is CH₂D.

69. The compound or stereoisomer or a pharmaceutically acceptable salt of any one of claims 50-65, wherein R₂ is CH₃.

70. The compound or stereoisomer or a pharmaceutically acceptable salt of claim 50, wherein the compound of Formula (II) is selected from the group consisting of:

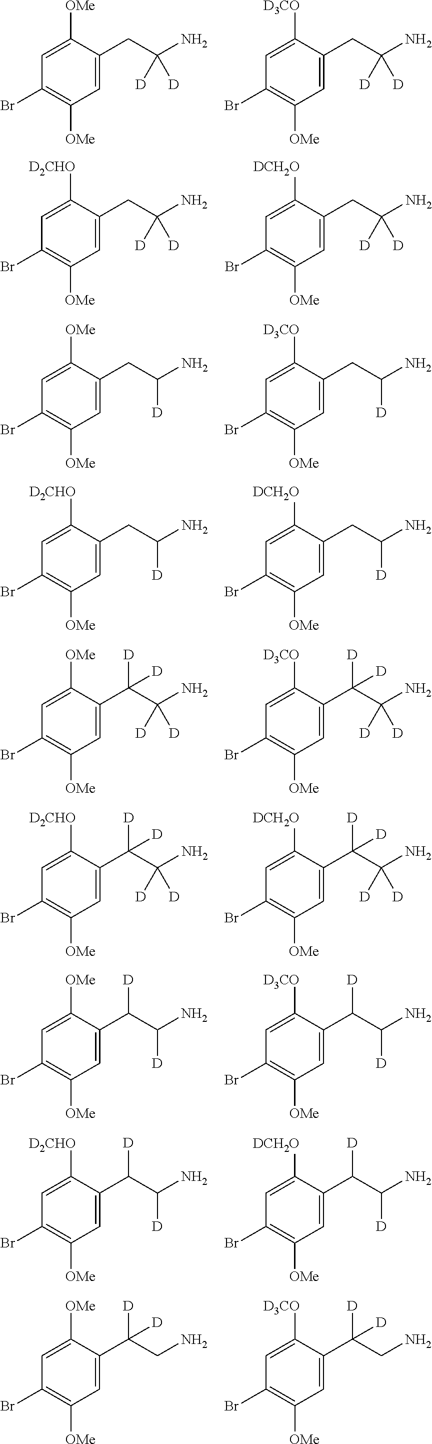

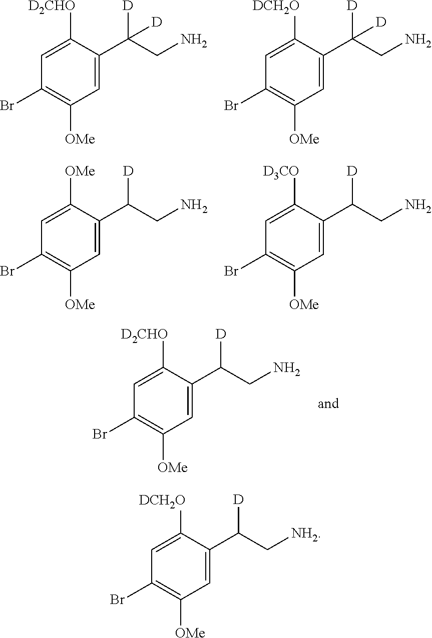

71. The compound or stereoisomer or a pharmaceutically acceptable salt of claim 50, wherein the compound of Formula (II) is a compound listed in Table 2.

72. The compound or stereoisomer or a pharmaceutically acceptable salt of claim 71, wherein the compound of Formula (II) is

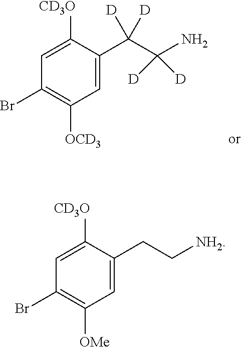

73. The compound or stereoisomer or a pharmaceutically acceptable salt of claim 1, wherein the compound of Formula (I) is of Formula (IIm): 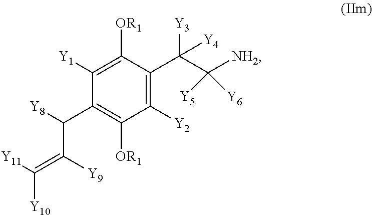
wherein
each R₁ is selected from CH₃, CH₂D, CHD₂, and CD₃;
Y₁, Y₂, Y₃, Y₄, Y₅, Y₆, Y₈, Y₉, Y₁₀, and Y₁₁, when present, are each independently H or deuterium; and
wherein at least one R₁ comprises one or more deuterium, or at least one of Y₁, Y₂, Y₃, Y₄, Y₅, Y₆, Y₈, Y₉, Y₁₀, and Y₁₁ is deuterium.

74. The compound or stereoisomer or a pharmaceutically acceptable salt of claim 1, wherein the compound of Formula (I) is of Formula (IIm′): 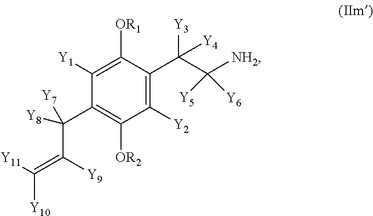
wherein
R₁ and R₂ are independently selected from CH₃, CH₂D, CHD₂, and CD₃;
Y₁, Y₂, Y₃, Y₄, Y₅, Y₆, Y₇, Y₈, Y₉, Y₁₀, and Y₁₁, when present, are each independently H or deuterium; and
wherein at least one of R₁ and R₂ comprises one or more deuterium, or at least one of Y₁, Y₂, Y₃, Y₄, Y₅, Y₆, Y₇, Y₈, Y₉, Y₁₀, and Y₁₁ is deuterium.

75. The compound or stereoisomer or a pharmaceutically acceptable salt of claim 74, wherein at least one of R₁ and R₂ comprises one or more deuterium.

76. The compound or stereoisomer or a pharmaceutically acceptable salt of claim 74, wherein R₁ comprises one or more deuterium.

77. The compound or stereoisomer or a pharmaceutically acceptable salt of claim 74, wherein R₂ comprises one or more deuterium.

78. The compound or stereoisomer or a pharmaceutically acceptable salt of claim 74, wherein both R₁ and R₂ comprise one or more deuterium.

79. The compound or stereoisomer or a pharmaceutically acceptable salt of claim 74, wherein both R₁ and R₂ do not comprise deuterium, and at least one of Y₁, Y₂, Y₃, Y₄, Y₅, Y₆, Y₇, Y₈, Y₉, Y₁₀, and Y₁₁ is deuterium.

80. The compound or stereoisomer or a pharmaceutically acceptable salt of any one of claims 74-79, wherein at least 2, 3, 4, 5, 6, 7, 8, 9, 10, or 11 of Y₁, Y₂, Y₃, Y₄, Y₅, Y₆, Y₇, Y₈, Y₉, Y₁₀, and Y₁₁ are deuterium.

81. The compound or stereoisomer or a pharmaceutically acceptable salt of any one of claims 74-79, wherein Y₅ and Y₆ are each deuterium, and Y₁, Y₂, Y₃, Y₄, Y₇, Y₈, Y₉, Y₁₀, and Y₁₁ are each hydrogen.

82. The compound or stereoisomer or a pharmaceutically acceptable salt of any one of claims 65-70, wherein Y₅ is deuterium, and Y₁, Y₂, Y₃, Y₄, Y₆, Y₇, Y₈, Y₉, Y₁₀, and Y₁₁ are each hydrogen.

83. The compound or stereoisomer or a pharmaceutically acceptable salt of any one of claims 65-70, wherein Y₃, Y₄, Y₅, and Y₆ are each deuterium, and Y₁, Y₂, Y₇, Y₈, Y₉, Y₁₀, and Y₁₁ are each hydrogen.

84. The compound or stereoisomer or a pharmaceutically acceptable salt of any one of claims 74-79, wherein Y₃ and Y₆ are each deuterium, and Y₁, Y₂, Y₄, Y₅, Y₇, Y₈, Y₉, Y₁₀, and Y₁₁ are each hydrogen.

85. The compound or stereoisomer or a pharmaceutically acceptable salt of any one of claims 74-79, wherein Y₃ and Y₄ are each deuterium, and Y₁, Y₂, Y₅, Y₆, Y₇, Y₈, Y₉, Y₁₀, and Y₁₁ are each hydrogen.

86. The compound or stereoisomer or a pharmaceutically acceptable salt of any one of claims 74-79, wherein Y₃ is deuterium, and Y₁, Y₂, Y₄, Y₅, Y₆, Y₇, Y₈, Y₉, Y₁₀, and Y₁₁ are each hydrogen.

87. The compound or stereoisomer or a pharmaceutically acceptable salt of any one of claims 74-79, wherein Y₁, Y₂, Y₃, Y₄, Y₅, and Y₆ are each deuterium, and Y₇, Y₈, Y₉, Y₁₀, and Y₁₁ are each hydrogen.

88. The compound or stereoisomer or a pharmaceutically acceptable salt of any one of claims 74-79, wherein Y₁, Y₂, Y₅, and Y₆ are each deuterium, and Y₃, Y₄, Y₇, Y₈, Y₉, Y₁₀, and Y₁₁ are each hydrogen.

89. The compound or stereoisomer or a pharmaceutically acceptable salt of any one of claims 74-79, wherein Y₅, Y₆, Y₇, Y₈, Y₉, Y₁₀, and Y₁₁ are each deuterium, and Y₁, Y₂, Y₃, and Y₄ are each hydrogen.

90. The compound or stereoisomer or a pharmaceutically acceptable salt of any one of claims 74-79, wherein Y₅, Y₆, Y₇, and Y₈, are each deuterium, and Y₁, Y₂, Y₃, Y₄, Y₉, Y₁₀, and Y₁₁ are each hydrogen.

91. The compound or stereoisomer or a pharmaceutically acceptable salt of any one of claims 74-79, wherein Y₃, Y₄, Y₅, Y₆, Y₇, Y₈, Y₉, Y₁₀, and Y₁₁ are each deuterium, and Y₁ and Y₂ are each hydrogen.

92. The compound or stereoisomer or a pharmaceutically acceptable salt of any one of claims 74-79, wherein Y₃, Y₄, Y₅, Y₆, Y₇, and Y₈, are each deuterium, and Y₁, Y₂, Y₉, Y₁₀, and Y₁₁ are each hydrogen.

93. The compound or stereoisomer or a pharmaceutically acceptable salt of any one of claims 74-79, wherein Y₆, Y₇, Y₈, Y₉, Y₁₀, and Y₁₁ are each deuterium, and Y₁, Y₂, Y₃, Y₄ and Y₅ are each hydrogen.

94. The compound or stereoisomer or a pharmaceutically acceptable salt of any one of claims 74-79, wherein Y₆, Y₇, and Y₈, are each deuterium, and Y₁, Y₂, Y₃, Y₄, Y₅, Y₉, Y₁₀, and Y₁₁ are each hydrogen.

95. The compound or stereoisomer or a pharmaceutically acceptable salt of any one of claims 74-79, wherein Y₃, Y₆, Y₇, Y₈, Y₉, Y₁₀, and Y₁₁ are each deuterium, and Y₁, Y₂, Y₅, and Y₄ are each hydrogen.

96. The compound or stereoisomer or a pharmaceutically acceptable salt of any one of claims 74-79, wherein Y₃, Y₆, Y₇, and Y₈, are each deuterium, and Y₁, Y₂, Y₄, Y₅, Y₉, Y₁₀, and Y₁₁ are each hydrogen.

97. The compound or stereoisomer or a pharmaceutically acceptable salt of any one of claims 74-79, wherein Y₃, Y₄, Y₇, Y₈, Y₉, Y₁₀, and Y₁₁ are each deuterium, and Y₁, Y₂, Y₅, and Y₆ are each hydrogen.

98. The compound or stereoisomer or a pharmaceutically acceptable salt of any one of claims 74-79, wherein Y₃, Y₄, Y₇, and Y₈, are each deuterium, and Y₁, Y₂, Y₅, Y₆, Y₉, Y₁₀, and Y₁₁ are each hydrogen.

99. The compound or stereoisomer or a pharmaceutically acceptable salt of any one of claims 74-79, wherein Y₃, Y₇, Y₈, Y₉, Y₁₀, and Y₁₁ are each deuterium, and Y₁, Y₂, Y₄, Y₅, and Y₆ are each hydrogen.

100. The compound or stereoisomer or a pharmaceutically acceptable salt of any one of claims 74-79, wherein Y₃, Y₇, and Y₈, are each deuterium, and Y₁, Y₂, Y₄, Y₅, Y₆, Y₉, Y₁₀, and Y₁₁ are each hydrogen.

101. The compound or stereoisomer or a pharmaceutically acceptable salt of any one of claims 74-79, wherein Y₁, Y₂, Y₅, Y₆, Y₇, Y₈, Y₉, Y₁₀, and Y₁₁ are each deuterium, and Y₃, Y₄ are each hydrogen.

102. The compound or stereoisomer or a pharmaceutically acceptable salt of any one of claims 74-79, wherein Y₁, Y₂, Y₅, Y₆, Y₇, and Y₈, are each deuterium, and Y₃, Y₄, Y₉, Y₁₀, and Y₁₁ are each hydrogen.

103. The compound or stereoisomer or a pharmaceutically acceptable salt of any one of claims 74-79, wherein Y₁, Y₂, Y₃, Y₄, Y₅, Y₆, Y₇, and Y₈, are each deuterium, and Y₉, Y₁₀, and Y₁₁ are each hydrogen.

104. The compound or stereoisomer or a pharmaceutically acceptable salt of any one of claims 74-79, wherein Y₁, Y₂, Y₃, Y₄, Y₅, Y₆, Y₇, Y₈, Y₉, Y₁₀, and Y₁₁ are each deuterium.

105. The compound or stereoisomer or a pharmaceutically acceptable salt of any one of claims 74 and 81-104, wherein R₁ is CD₃.

106. The compound or stereoisomer or a pharmaceutically acceptable salt of any one of claims 74 and 81-104, wherein R₁ is CHD₂.

107. The compound or stereoisomer or a pharmaceutically acceptable salt of any one of claims 74 and 81-104, wherein R₁ is CH₂D.

108. The compound or stereoisomer or a pharmaceutically acceptable salt of any one of claims 74 and 81-107, wherein R₂ is CH₃.

109. The compound or stereoisomer or a pharmaceutically acceptable salt of claim 74, wherein the compound of Formula (IIm′) is selected from the group consisting of:

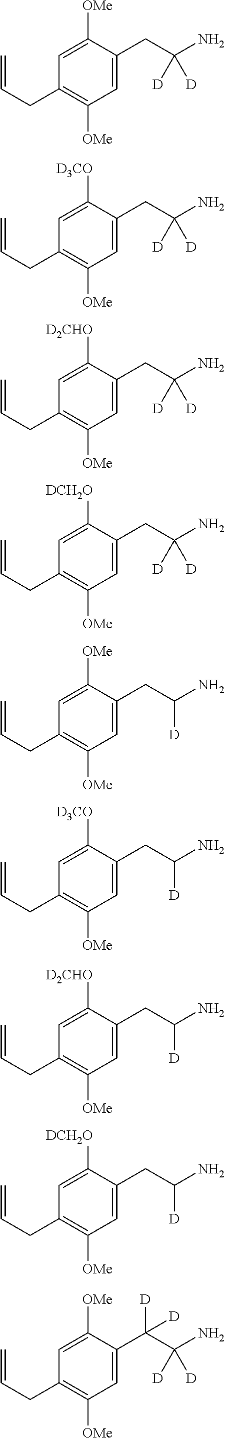

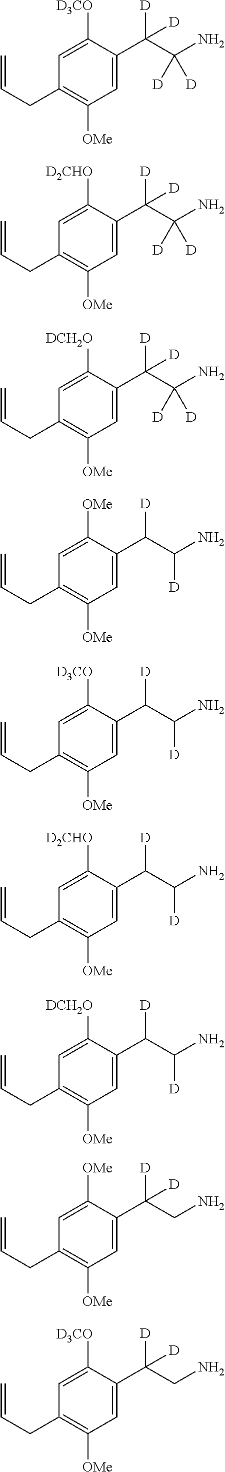

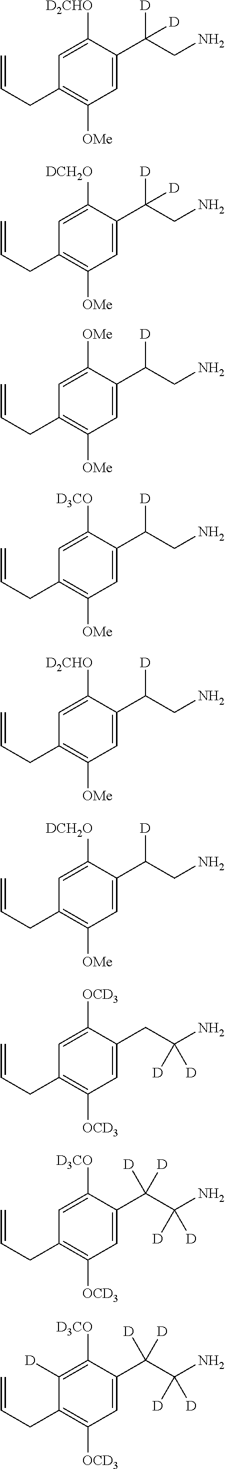

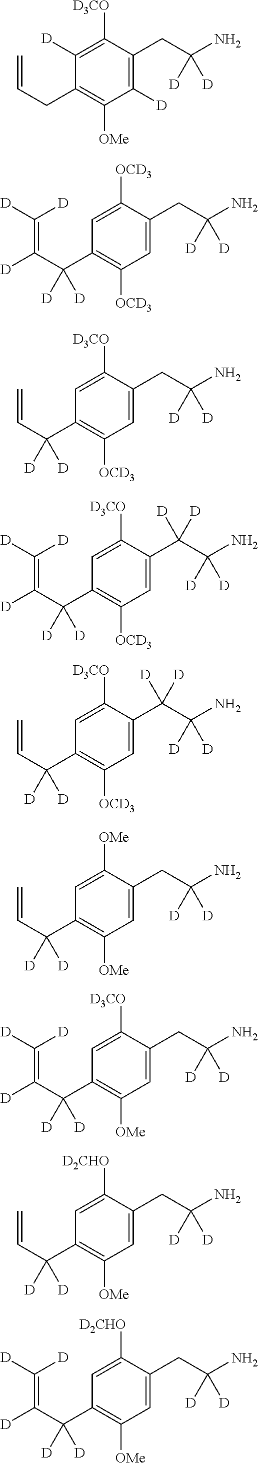

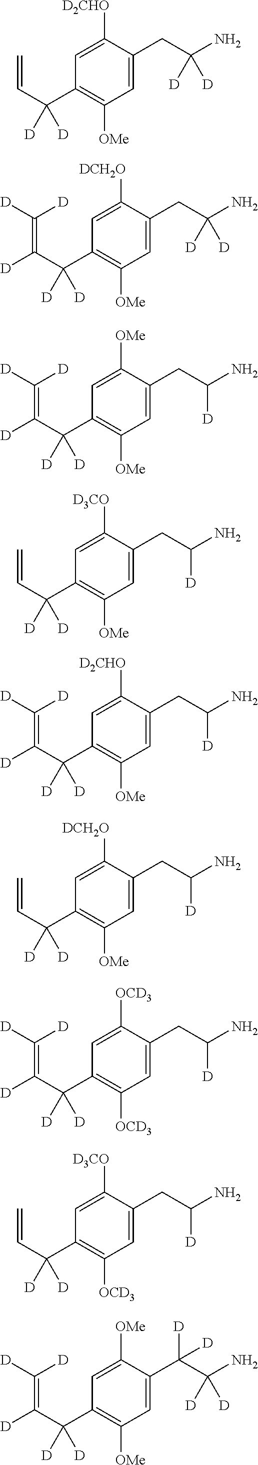

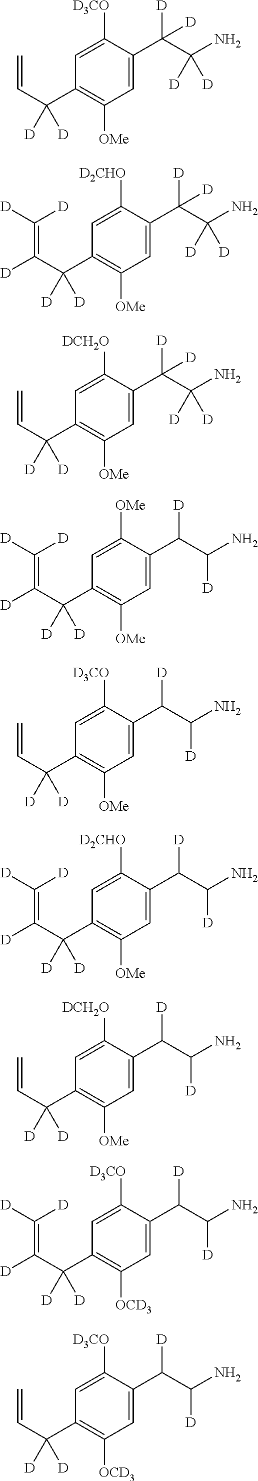

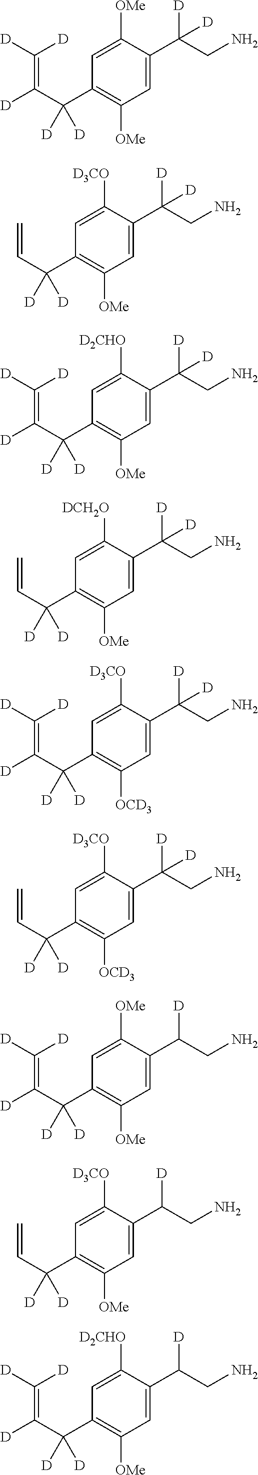

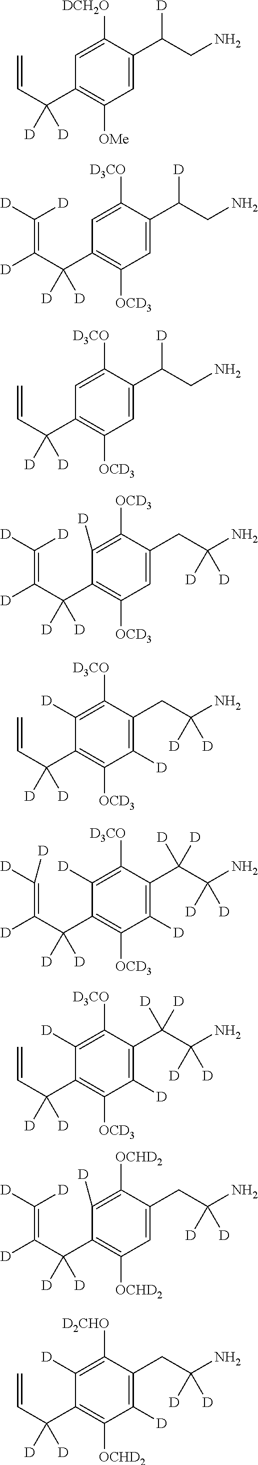

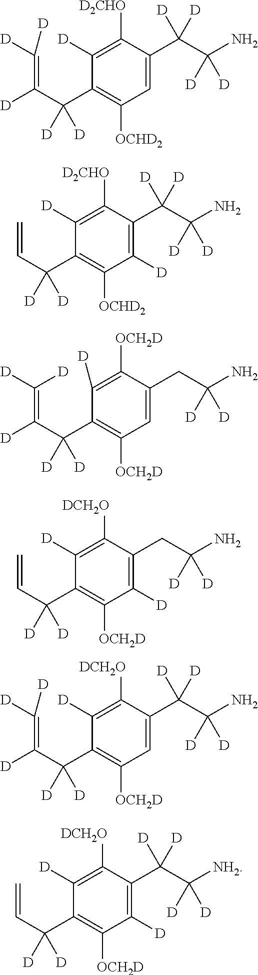

110. The compound or stereoisomer or a pharmaceutically acceptable salt of claim 1, the compound has a formula of: 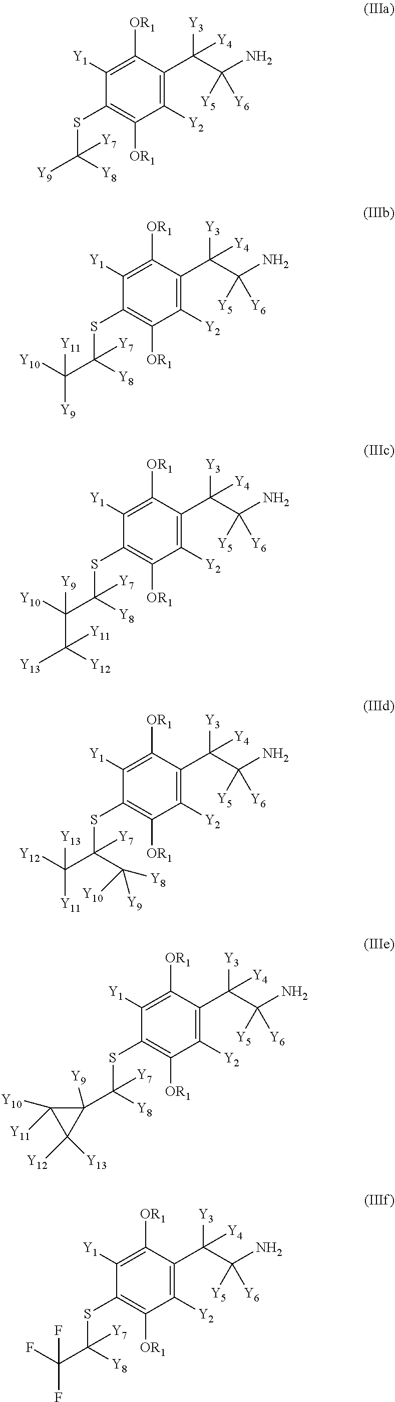 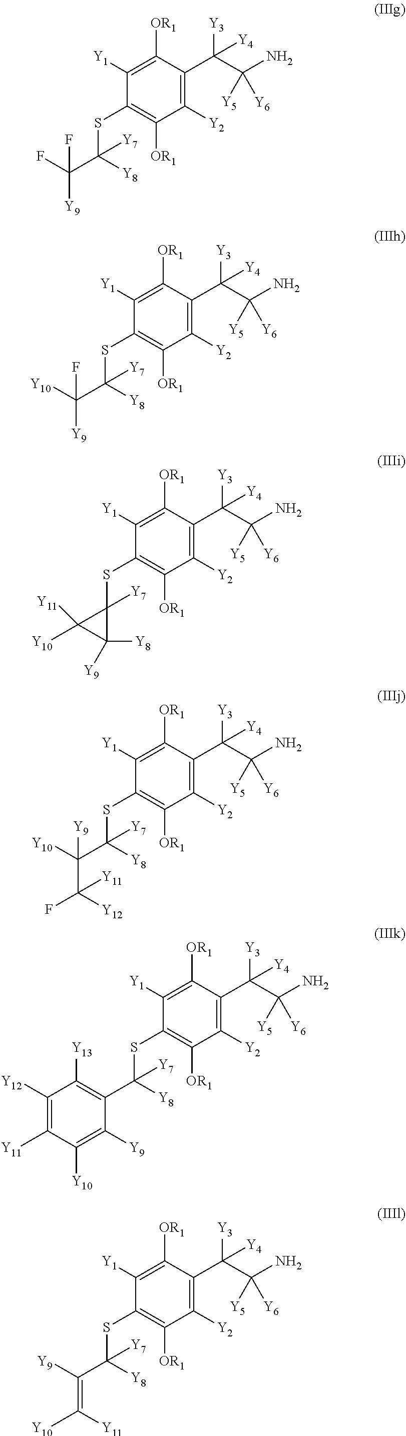 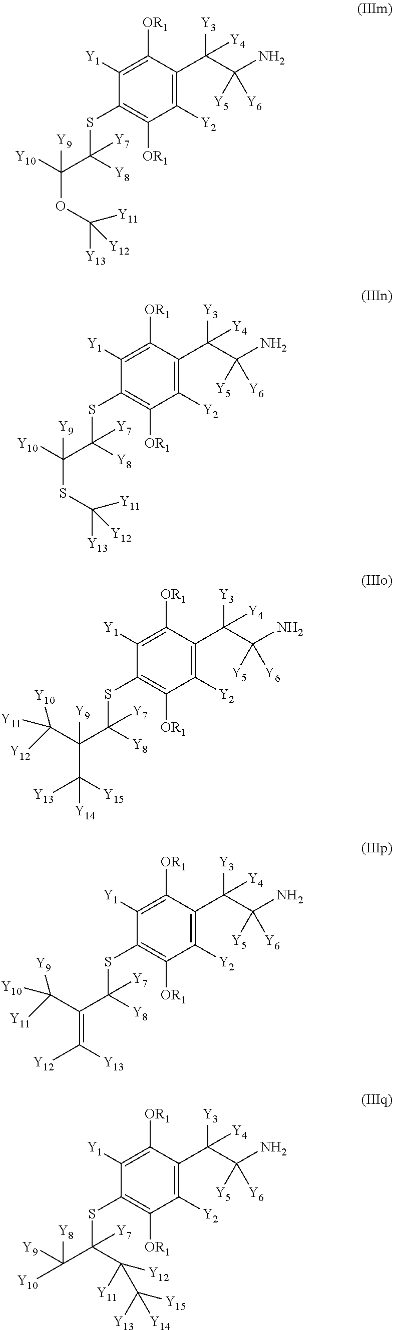 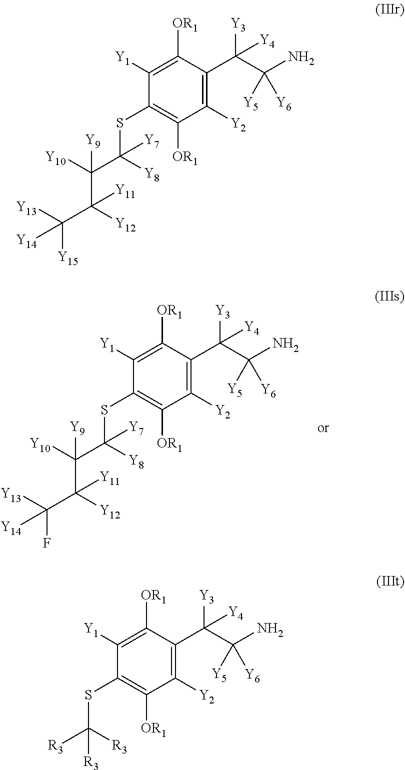
wherein
each R₁ is independently selected from CH₃, CH₂D, CHD₂, and CD₃;
each R₃ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, are each independently H or deuterium; and
wherein at least one of R₁ and R₃ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, and Y₁₅, when present, is deuterium.

111. The compound or stereoisomer or a pharmaceutically acceptable salt of claim 1, the has a formula of: 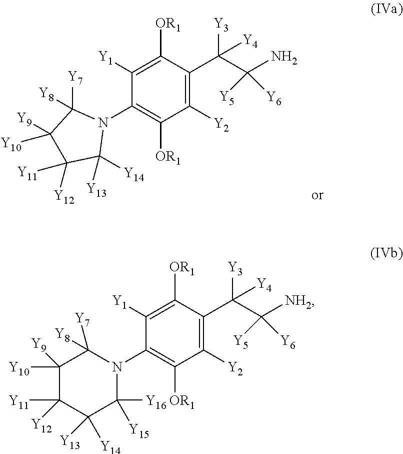
each R₁ is independently selected from CH₃, CH₂D, CHD₂, and CD₃;
Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆ are each independently H or deuterium; and wherein at least one R₁ comprises one or more deuterium, or at least one of Y₁, Y₂, Y₃, Y₄, Y₅, Y₆, Y₇, Y₈, Y₉, Y₁₀, Y₁₁, Y₁₂, Y₁₃, Y₁₄, Y₁₅, and Y₁₆, when present, is deuterium.

112. A pharmaceutical composition comprising the compound or stereoisomer or a pharmaceutically acceptable salt according to any one of claims 1-111, and a pharmaceutically acceptable excipient or carrier.

113. A method for treating or preventing a disease, disorder, or condition a subject in need thereof, comprising administering to the subject an effective amount of the compound or stereoisomer or a pharmaceutically acceptable salt according to any one of claims 1-111, or the pharmaceutical composition according to claim 112.

114. The method of claim 113, wherein the disease, disorder, or condition is a brain disease or disorder.

115. The method of claim 113 or 114, wherein the brain disease or disorder is a neurodegenerative disorder.

116. The method of any one of claims 113-115, wherein the brain disease or disorder is psychological disorder, depression, addiction, anxiety, or a post-traumatic stress disorder.

117. A method for increasing neuronal plasticity in a subject in need thereof, comprising administering to the subject an effective amount of the compound or stereoisomer or a pharmaceutically acceptable salt according to any one of claims 1-111, or the pharmaceutical composition according to claim 112.
